(19) United States
(12) Patent Application Publication
(10) Pub. No.: US 2023/0332213 A1
(43) Pub. Date: Oct. 19, 2023

(54) IMPROVED HIGH EFFICIENCY TARGETED IN SITU GENOME-WIDE PROFILING

(71) Applicant: Fred Hutchinson Cancer Center, Seattle, WA (US)

(72) Inventor: Steven Henikoff, Seattle, WA (US)

(21) Appl. No.: 18/044,289

(22) PCT Filed: Sep. 10, 2021
(86) PCT No.: PCT/US21/49944

§ 371 Date: Mar. 7, 2023

Related U.S. Application Data
(60) Provisional application No. 63/077,496, filed on Sep. 11, 2020.
(60) Provisional application No. 63/196,953, filed on Jun. 4, 2021.

(51) Int. Cl. C12Q 1/6841; C12Q 1/6858

(52) U.S. Cl. CPC C12Q 1/6841; C12Q 1/6858; C12Q 2600/166; C12Q 1/6806

(57) ABSTRACT

The disclosure provides methods and related systems and reagents for detecting, sequencing, and/or mapping sites of DNA accessibility in the chromatin of a cell. The method comprises contacting a permeabilized cells with an affinity reagent that specifically binds a nucleosome depleted region (NDR) marker. The first affinity reagent is coupled, directly or indirectly, with at least one transposome. The transposase component of the transposome is activated under low ionic conditions, resulting in cleaving and tagging chromatin DNA. The DNA segment, thus tagged, is excised by virtue of multiple cleavage points, which is then isolated for analysis (e.g., sequencing and mapping). The method can include additional affinity reagents that are similarly functionalized but instead bind to negative regulatory elements in the chromatin, thus allowing for the simultaneous mapping of DNA accessibility and inaccessibility in the genome of a single cell. The methods can be applied to a variety of analytic platforms.

### CROSS-REFERENCES TO RELATED APPLICATIONS

This application claims the benefit of U.S. Provisional Application Nos. 63/077,496, filed Sep. 11, 2020, and 63/196,953, filed Jun. 4, 2021, the disclosures of which are hereby expressly incorporated herein by reference in their entireties.

### BACKGROUND

Identification of DNA accessibility in the chromatin landscape has been used to infer active transcription ever since the seminal description of DNaseI hypersensitivity by Weintraub and Groudine more than 40 years ago. Because nucleosomes occupy most of the eukaryotic chromatin landscape and regulatory elements are on average free of nucleosomes when they are active, DNA accessibility mapping can potentially identify active regulatory elements genome-wide. Several strategies have been introduced to identify regulatory elements by DNA accessibility mapping, including digestion with Micrococcal Nuclease (MNase) or restriction enzymes, physical fragmentation and transposon insertion. With the advent of genome-scale mapping platforms, beginning with microarrays and later short-read DNA sequencing, mapping regulatory elements based on DNaseI hypersensitivity became routine. Later innovations included FAIRE and Sono-seq, based on physical fragmentation and differential recovery of cross-linked chromatin, and ATAC-seq, based on preferential insertion of the Tn5 transposase. The speed and simplicity of ATAC-seq, in which the cut-and-paste transposition reaction inserts sequencing adapters in the most accessible genomic regions (tagmentation), has led to its widespread adoption in many laboratories for mapping presumed regulatory elements.

For all of these DNA accessibility mapping strategies, it is generally unknown what process is responsible for creating any particular accessible sites within the chromatin landscape. Furthermore, accessibility is not all-or-none, with the median difference between an accessible and a non-accessible site in DNA estimated to be only ˜20%, with no sites completely accessible or inaccessible in a population of cells. Despite these uncertainties, DNA accessibility mapping has successfully predicted the locations of active gene enhancers and promoters genome-wide, with excellent correspondence between methods based on very different strategies. This is likely because DNA accessibility mapping strategies rely on the fact that nucleosomes have evolved to repress transcription by blocking sites of pre-initiation complex formation and transcription factor binding, and so creating and maintaining a nucleosome-depleted region (NDR) is a pre-requisite for promoter and enhancer function.

A popular alternative to DNA accessibility mapping for regulatory element identification is to map nucleosomes that border NDRs, typically by histone marks, including “active” histone modifications, such as H3K4 methylation and H3K27 acetylation, or histone variants incorporated during transcription, such as H2A.Z and H3.3. The rationale for this mapping strategy is that the enzymes that modify histone tails and the chaperones that deposit nucleosome subunits are most active close to the sites of initiation of transcription, which typically occurs bidirectionally at both gene promoters and enhancers to produce stable mRNAs and unstable enhancer RNAs. Although the marks left behind by active transcriptional initiation “point back” to the NDR, this cause-effect connection between the NDR and the histone marks is only by inference, and direct evidence is lacking that a histone mark is associated with an NDR.

Accordingly, despite the advances in the art, there remains a need for facile and accurate analyses to identify active regulatory elements across a genome. This disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method for detecting a site of DNA accessibility in the chromatin of a cell. The method comprises:
contacting a permeabilized cell (or nucleus) with a first affinity reagent that specifically binds a nucleosome depleted region (NDR) marker, wherein the first affinity reagent is coupled to at least one transposome comprising:
at least one transposase; and
a transposon comprising:
   a first DNA molecule comprising a first transposase recognition site; and
   a second DNA molecule comprising a second transposase recognition site;
activating the at least one transposase under low ionic conditions, thereby cleaving and tagging chromatin DNA with the first and second DNA molecules and excising a tagged DNA segment associated with the NDR marker;
isolating the excised tagged DNA segment; and
determining the nucleotide sequence of the excised tagged DNA segment, thereby detecting the site of DNA accessibility in the chromatin of the cell.

In some embodiments, the first affinity reagent is directly coupled to at least one transposase. In some embodiments, the first affinity reagent and transposase are disposed in a fusion protein. In some embodiments, the first affinity reagent is indirectly coupled to the at least one transposase. In some embodiments, the transposase is linked to a specific binding agent that specifically binds the first affinity reagent. In some embodiments, the method further comprises contacting the cell with a second affinity reagent that specifically binds the first affinity reagent, and wherein the transposase is linked to a specific binding agent that specifically binds the second affinity reagent. In some embodiments, the method further comprises contacting the cell with a second affinity reagent that specifically binds the first affinity reagent, contacting the cell with a third affinity reagent that specifically binds the second affinity reagent, and wherein the transposase is linked to a specific binding agent that specifically binds the third affinity reagent. In some embodiments, the specific binding agent comprises protein A, protein G, protein L, protein Y, or binding domains thereof, or a fourth affinity reagent that specifically binds the first affinity reagent, the second affinity reagent and/or the third affinity reagent. In some embodiments, the first, second, and/or third affinity reagents independently is or comprises an antibody, an antibody-like molecule, a DARPin, an aptamer, a chromatin-binding protein, other specifically binding molecule, or a functional antigen-binding domain thereof. In some embodiments, the antibody-like molecule is an antibody fragment and/or antibody derivative. In some embodiments, the antibody-like molecule is a single chain antibody, a bispecific antibody, an Fab fragment, an F(ab)2 fragment, a VHH fragment, a VNAR fragment, or a nanobody. In some embodiments, the single-chain antibody is a single chain variable fragment (scFv), or a single-chain Fab fragment (scFab).

In some embodiments, the low ionic conditions are characterized by monovalent ionic concentration of less than about 10 mM. In some embodiments, the low ionic conditions are obtained by diluting liquid conditions of the transposase with a Mg++ solution, removing liquid supernatant from the transposase and replacing it with a low ionic strength solution, and/or conducting a stringent (e.g., 300 mM) wash followed by adding a low ionic strength solution. In some embodiments, the method further comprises contacting the permeabilized cell with a polar compound prior to or during the step of activating the transposase under low ionic conditions. In some embodiments, the polar compound is 1,6-hexanediol or N,N dimethylformamide. In some embodiments, the cell is immobilized on a solid surface. In some embodiments, the solid surface comprises a bead or wall of a microtiter plate.

In some embodiments, the first and/or second DNA molecule further comprises a barcode. In some embodiments, the first and/or second DNA molecule further comprises a sequencing adaptor. In some embodiments, the first and/or second DNA molecule further comprises a universal priming site.

In some embodiments, the at least one transposase comprises a Tn5 transposase. In some embodiments, activating the transposase under low ionic conditions comprises contacting the transposase with Mg++, optionally with about 0.1 mM Mg++ to about 10 mM Mg++. In some embodiments, the at least one transposase comprises a Mu transposase. In some embodiments, the at least one transposase comprises an IS5 or an IS91 transposase.

In some embodiments, the least one transposome comprises at least two different transposases, and wherein the different transposases integrate different DNA sequences into the chromatin DNA. In some embodiments, the method is performed with a plurality of first affinity reagents, thereby producing a plurality of excised tagged DNA segments, and wherein the method further comprises isolating a plurality of excised tagged DNA segments.

In some embodiments, the method further comprises analyzing the isolated tagged DNA segments. In some embodiments, analyzing the isolated tagged DNA segments comprises determining the nucleotide sequence of the tagged DNA segments. In some embodiments, the nucleotide sequence is determined using sequencing or hybridization techniques with or without amplification.

In some embodiments, the cell is a eukaryote cell, such as a human cell. In some embodiments, the cell and/or the nucleus of the cell is permeabilized by contacting the cell with digitonin.

In some embodiments, the method further comprises subjecting the excised DNA to salt fractionation.

In some embodiments, the NDR marker is a histone modification, optionally methylated H3K4, optionally wherein methylated H3K4 is bi-methylated or tri-methylated.

In some embodiments, the NDR marker is an initiating form of RNA Polymerase II, optionally serine 5-phosphorylated RNA Polymerase II (RNAPIIS5P) or serine 2-phosphorylated RNA Polymerase II (RNAPIIS2P).

In some embodiments, the method further comprises contacting the permeabilized cell with a known amount of spike-in DNA configured to facilitate calibration. In some embodiments, the spike-in DNA is or comprises exogenous DNA, exogenous chromatin, or recombinant nucleosomes. In some embodiments, the first affinity reagent is coupled to a plurality of transposomes, a fraction of the plurality of transposomes comprising a known amount of spike-in DNA, and wherein the spike-in DNA can be used for calibration.

In some embodiments, the at least one transposome comprises a fusion protein comprising a first domain comprising a Tn5 transposase domain and second domain comprising a protein A domain, a protein G domain, a protein L domain, a protein Y domain, hybrid thereof in any combination (e.g., a protein A domain/protein G domain hybrid).

In some embodiments, the method is performed for a plurality of cells and the method further comprises mapping the determined sequences of one or more excised tagged DNA segments to a consensus genome of the plurality of the cells. In some embodiments, the method further comprises mapping the determined sequence of the excised tagged DNA segment to the genome of the cell. In some embodiments, the method is performed for a plurality of cells, wherein the excised tagged DNA segments of each of the plurality of cells is tagged with a cell-specific barcode or combination of barcodes that is unique to each cell. In some embodiments, the method further comprises application of combinatorial indexing to provide the cell-specific barcode or combination of barcodes to the excised tagged DNA segments of each of the plurality of cells. In some embodiments, the plurality of cells is disposed in a three-dimensional arrangement and the cell-specific barcode or combination of barcodes is unique to a location in the three-dimensional arrangement. In some embodiments, the three-dimensional arrangement is a tissue slice or tissue culture array.

In another aspect, the disclosure provides a method of detecting active and repressive regulomes in a cell, comprising performing the CUTAC method described herein, wherein the method further comprises contacting the permeabilized cell with the first affinity reagent in combination with a fifth affinity reagent that specifically binds a repressive regulatory element marker. Like the first affinity reagent, the fifth affinity reagent is coupled to at least one transposome comprising:
at least one transposase; and
a transposon comprising:
   a first DNA molecule comprising a first transposase recognition site; and
   a second DNA molecule comprising a second transposase recognition site;
activating the at least one transposase under low ionic conditions, thereby cleaving and tagging chromatin DNA with the first and second DNA molecules and excising a tagged DNA segment associated with the repressive regulatory element marker;
isolating the excised tagged DNA segment associated with the repressive regulatory element marker;
determining the sequence of the excised tagged DNA segment associated with the repressive regulatory element marker; and
deconvoluting the sequences determined from the excised tagged DNA segment associated with the NDR marker and the excised tagged DNA segment associated with the repressive regulatory marker, thereby detecting active and repressive regulomes in the cell.

In some embodiments, the repressive regulatory element marker is a methylated histone, optionally methylated, H3K27, optionally, wherein methylated H3K27 is tri-methylated. In some embodiments, a plurality of sequences is determined from a plurality of excised tagged DNA segments associated with the NDR marker and a plurality of excised tagged DNA segments associated with the repressive regulatory marker, and wherein the sequences are deconvoluted based on different tagmentation densities and/or different fragment sizes associated with the NDR marker and the repressive regulatory marker.

In another aspect, the disclosure provides methods for preparing a library of excised chromatin DNA, comprising the steps disclosed herein, e.g., for the disclosed CUTAC and/or CUT&Tag2for1 methods.

In another aspect, the disclosure provides a kit, comprising one or more of: the first affinity reagent, the second affinity reagent, the third affinity reagent, the fourth affinity reagent, the fifth affinity reagent, the transposase (e.g., comprising a Tn5 domain), the specific binding agent, the polar compound, the solid surface (e.g., bead or microtiter plate), the Mg++ solution, a low ionic strength solution, the stringent wash solution, buffers, and other reagents to facilitate performance of a method as described herein, and optionally written indicia directing the performance the method as described herein.

In some embodiments, the kit further comprises a high ionic solution and a low ionic solution to provide high ionic conditions and ionic conditions for transposase activity in parallel containers.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1D: CUT&Tag-direct produces high-quality datasets on the benchtop and at home. Starting with a frozen human K562 cell aliquot, CUT&Tag-direct with amplification for 12 cycles yields detectable nucleosomal ladders for intermediate and low numbers of cells for both (1A) H3K4me3 and (1B) H3K27me3. (1C) Comparison of H3K4me3 CUT&Tag-direct results produced in the laboratory to those produced at home and to an ENCODE dataset (GSM733680). (1D) Same as (1C) for H3K27me3 comparing CUT&Tag-direct results to CUT&Tag datasets using the standard protocol (Kaya-Okur H S, et al. (2019) CUT&Tag for efficient epigenomic profiling of small samples and single cells. Nat Commun 10:1930), and to an ENCODE dataset (GSM788088). pA-Tn5 was used except as indicated by asterisks for datasets produced at home using commercial pAG-Tn5 (Epicypher cat. no. 15-1017).

FIGS. 2A-2G: Low-salt tagmentation of H3Kme2/3 CUT&Tag samples sharpen peaks. (2A) Tapestation gel image showing the change in size distribution from standard CUT&Tag (CnT), tagmented in the presence of 300 mM NaCl with low-salt tagmentation using the dilution protocol. (2B) Representative tracks showing the shift observed with low-salt dilution tagmentation. (2C) Average plots showing the narrowing of peak distributions upon low-salt tagmentation using the dilution protocol. (2D) Heatmaps showing narrowing of H3K4me2 peaks after removing pAG-Tn5 (Removal), after a stringent wash (Post-wash), and after a stringent wash with low-salt tagmentation including a 1% pAG-Tn5 spike-in (Add-back). (2E) Heatmaps showing dilution tagmentation and further narrowing of H3K4me2 peak distributions upon low-salt tagmentation (after removal) for 20 minutes at 37° C. in the presence of 10% 1,6-hexanediol (hex) and 10% dimethylformamide (DMF) or both for 1 hr at 55° C. (2F) Average plots showing effects of tagmentation with hex and/or DMF over time of low-salt tagmentation (after removal). (2G) Smaller fragments (≤120 bp) dominate NDRs.

FIGS. 3A-3D: H3K4me2 CUTAC sites correspond to ATAC-seq sites. Heatmaps showing the correspondence between CUTAC and ATAC-seq sites. Headings over each heatmap denote the source of mapped fragments mapping to the indicated set of MACS2 peak summits, ordered by occupancy over the 5 kb interval centered over each site. CUT&Tag and CUTAC sites are from samples processed in parallel, where CUTAC tagmentation was performed by 20-fold dilution and 20 minute 37° C. incubation following pAG-Tn5 binding.

FIGS. 4A-4C: CUTAC data quality is similar to the best available ATAC-seq K562 cell data. Mapped fragments from the indicated datasets were sampled and mapped to hg19 using Bowtie2, and peaks were called using MACS2. (4A) Number of peaks (left) and fraction of reads in peaks (right) for CUT&Tag (blue), CUTAC (red) and ATAC-seq (green). Fast-ATAC is an improved version of ATAC-seq that reduces mitochondrial reads (Corces M R, et al. (2016) Lineage-specific and single-cell chromatin accessibility charts human hematopoiesis and leukemia evolution. Nat Genet 48:1193-1203), and Omni-ATAC is an improved version that additionally improved signal-to-noise (Corces M R, et al. (2017) An improved ATAC-seq protocol reduces background and enables interrogation of frozen tissues. Nat Methods 14:959-962). ATAC-seq_ENCODE is the current ENCODE standard (Moore J E, et al. (2020) Expanded encyclopaedias of DNA elements in the human and mouse genomes. Nature 583:699-710). (4B) Five other K562 ATAC-seq datasets from different laboratories were identified in GEO and mapped to hg19. MACS2 was used to call peaks and peak numbers and FRiP values indicate a wide range of data quality found in recent ATAC-seq datasets. Number of peaks (left) and fraction of reads in peaks (right). (4C) Small CUTAC fragments improved peak-calling. Number of peaks (left) and fraction of reads in peaks (right).

FIGS. 5A-5C: H3K4me2 CUTAC sites are coupled to transcription. (5A) H3K4me2 fragments shift from flanking nucleosomes to the NDR upon low-salt tagmentation, corresponding closely to ATAC-seq sites. (5B) The Serine-5 phosphate-marked initiation form of RNAPII is highly abundant over most H3K4me2 CUT&Tag, CUTAC and ATAC-seq peaks. (5C) Run-on transcription initiates from most sites corresponding to CUTAC and ATAC-seq peaks. Both plus and minus strand PRO-seq datasets downloaded from GEO (GSM3452725) were pooled and aligned over peaks called using 3.2 million fragments sampled from H3K4me2 CUT&Tag, CUTAC and Omni-ATAC datasets, and also from pooled CUT&Tag replicate datasets for K562 RNA Polymerase II Serine-5 phosphate.

FIG. 6: Low-salt tagmentation using various antibodies. Two H3K4me2 antibodies were used: Millipore 07-030 lot 3229364 (Mi) and Epicypher 13-0027 (Ep) and provided similar results. CUTAC was done using the Removal protocol and incubated 10 min at 37° C.

FIG. 7: Optimization of low-salt tagmentation conditions: H3K4me2 CUT&Tag and low-salt tagmentation were performed using either a rabbit polyclonal [Millipore 07-030 lot 3229364 (Mi)] or rabbit monoclonal [Epicypher 13-0027 (Ep)] antibody with pAG-Tn5 (Epicypher 15-1117 lot #20142001-C1) at the indicated dilutions. Dilution tagmentation in 2 mM MgCl₂ was used at either 22° C. or 37° C. Raw paired-end reads were sampled down to 3.2 million and mapped to hg19. A representative 100 kb region is shown (left) and expanded (right) around active promoters and group-autoscaled separately for low-salt tagmentation and standard CUT&Tag using IGV. Estimated library size (Lib size) was calculated by the Mark Duplicates program in Picard tools.

FIG. 8: Low-salt tagmentation is consistent in the presence of strongly polar compounds: H3K4me2 CUT&Tag and low-salt tagmentation using the removal protocol were performed at 37° C. using Epicypher 13-0027 antibody and Epicypher 15-1117 pAG-Tn5 for the times indicated. Raw paired-end reads were sampled down to 3.2 million and mapped to hg19. A representative 100-kb region is shown (left) and expanded (right) around active promoters and group-autoscaled using IGV. Estimated library size (Lib size) was calculated by the Mark Duplicates program in Picard tools.

FIG. 9: Smaller fragments (≤120 bp) dominate NDRs. Additional comparisons of small (≤120 bp) and large (>120) fragments from diverse CUTAC datasets used in this study show consistent narrowing for small fragments around their summits.

FIGS. 10A-10B: CUTAC data quality is similar to that of the best ATAC-seq datasets. 10A is a table showing human K562 and H1 ES cell ATAC-seq datasets that were downloaded from GEO, and Bowtie2 was used to map fragments to hg19. A sample of 3.2 million mapped fragments without Chr M was used for peak-calling by MACS2 to calculate FRiP values. Year of submission to GEO or SRA databanks is shown. % Chr M is percent of fragments mapped to Chr M (mitochondrial DNA). (10B) Tracks over a representative region for K562 datasets listed in (10A). Samples are ordered by decreasing FRiP.

FIG. 11: Small CUTAC fragments improved peak resolution.

FIGS. 12A and 12B: Overview of in situ tethering for CUT&Tag chromatin profiling, which forms the basis of CUTAC. (12A) The steps in CUT&Tag. Added antibody (10) binds to the target chromatin protein (20) between nucleosomes (30) in the genome, and the excess is washed away. A second antibody (40) is added and enhances tethering of pA-Tn5 transposome (50) at antibody-bound sites. After washing away excess transposome, addition of Mg++ activates the transposome and integrates adapters (60) at chromatin protein binding sites. After DNA purification genomic fragments with adapters at both ends are enriched by PCR. (12B) CUT&Tag is performed on a solid support. Unfixed cells (70) or nuclei (80) are permeabilized and mixed with antibody to a target chromatin protein. After addition and binding of cells to Concanavalin A-coated magnetic beads (M), all further steps are performed in the same reaction tube with magnetic capture between washes and incubations, including pA-Tn5 tethering, integration, and DNA purification.

FIGS. 13A and 13B: Schemes illustrating embodiments of the CUTAC approach. 13A illustrates a simplified scheme for simultaneous CUT&Tag and (H3K4me2 or RNAPIIS5P) CUTAC. CUT&Tag-direct is performed in nuclei in situ in single PCR tubes with Concanavalin A (ConA) bead-bound nuclei that remain intact throughout the protocol during successive liquid changes, incubations and washes, 12 cycles of PCR amplification and one SPRI bead clean-up. CUTAC is performed identically except that low-salt conditions are used for tagmentation. H3K4me2 CUTAC maps accessible sites near H3K4me2/3-marked (starred) nucleosome tails, which are methylated by the conserved Set1 lysine methyltransferase. The complex that includes Set1 associates with the initiation form of RNAPII, which is heavily phosphorylated on Serine-5 of the heptameric C-terminal domain repeat units on the largest RNAPII subunit (RNAPIIS5P). For RNAPIIS5P CUTAC, pA-Tn5 is anchored directly to RNAPIIS5 phosphates (starred). Whereas CUT&Tag is suitable for any chromatin epitope, CUTAC is specific for H3K4me2, H3K4me3 and RNAPIIS5P. The only other difference between the protocols is that tagmentation is performed in the presence of 300 mM NaCl for CUT&Tag and in a low ionic strength buffer for CUTAC. 13B is a schematic illustrating use of primary antibodies to bind the target epitope (e.g., α-H3K4me2) and secondary antibody binding to the primary antibody to associate with the Protein A-Tn5 complex to provide for amplification of the functional transposase activity. Exemplary differences between the CUT&Tag and CUTAC methods are illustrated.

FIG. 14: Tapestation profiles for a low-cell-number RNAPIIS5P CUTAC experiment. Tagmentation was performed for 20 min at 37° C. in CUTAC-hex buffer. Representative tracks for these samples are shown in FIG. 15A.

FIGS. 15A-15D: Accessible DNA corresponds to binding sites of the initiating form of RNA Polymerase II (RNAPII). (15A) Tracks show profiles of the Chromosome 1 histone gene cluster, with 12 small intronless genes expressed at high levels in all dividing cells. Whereas RNAPIIS5P CUT&Tag shows broad enrichment over each of the genes, the CUTAC protocol applied to the RNAPIIS5P epitope, either native (RNAPIIS5P CUTAC-N) or cross-linked (RNAPIIS5P CUTAC-X), yields sharp promoter delineation, better than H3K4me2 CUTAC with or without 1,6-hexanediol or the best K562 ATAC-seq datasets (Omni-ATAC, ATAC_{ENCODE}, Fast-ATAC), all downsampled to 3.2 million mapped fragments. Note the 10-fold difference in scale between RNAPIIS5P CUTAC (0-1500 and K4me2-CUTAC/ATAC (0-150). Similar results were obtained for three mixed-lineage leukemia cell lines (KOPN8, SEM and RS411) and H1 embryonic stem cells down to ˜2,000 cells. No changes were made to the protocol for low cell numbers. Numbers in parentheses are estimated library sizes in millions of mapped paired-end reads. (15B-15D) RNAPIIS5P occupies sites of accessible chromatin in K562 cells. (15B) Left to right: K4me2 CUT&Tag, K4me2 CUTAC, RNAPIIS5P CUT&Tag, RNAPIIS5P CUTAC, Omni-ATAC and Fast-ATAC datasets were downsampled to 3.2 million fragments and aligned over ATAC-seq peaks called using MACS2 on data generated by the ENCODE project (ATACENCODE). (15C) Same as (15A) except using only subnucleosome-sized fragments (≤120 bp). CUTAC RNAPIIS5P sites are virtually indistinguishable from high-quality ATAC-seq data, directly demonstrating that ATAC-seq maps sites of the initiation form of RNA Pol II. (15D) Same as (15A) except using only >120 bp fragments. ENCODE ATAC-seq fragments were downsampled to 3.2 million, ChrM (mitochondrial DNA) was removed and MACS2 was used to call peaks. Heatmaps are centered over ENCODE ATAC-seq peak summits and ordered by occupancy over the 5 kb span displayed. Fast-ATAC is an improved version of ATAC-seq that reduces mitochondrial reads (Corces, M. R., et al. (2016). Lineage-specific and single-cell chromatin accessibility charts human hematopoiesis and leukemia evolution. Nat Genet 48(10): 1193-1203), and Omni-ATAC is an improved version that additionally improves the signal-to-noise ratio (Corces, M. R., et al. (2017). An improved ATAC-seq protocol reduces background and enables interrogation of frozen tissues. Nat Methods 14(10): 959-962). ATAC_ENCODE is the current ENCODE standard (Consortium, E. P., et al. (2020). Expanded encyclopaedias of DNA elements in the human and mouse genomes. Nature 583(7818): 699-710).

FIGS. 16A and 16B: RNAPIIS5P CUTAC shows high sensitivity and specificity. Mapped fragments from the indicated K562 datasets were sampled, and peaks were called using MACS2. (16A) Number of peaks and (16B) fraction of reads in peaks for CUT&Tag (triangles) and CUTAC (squares) profiles for H3K4me2, RNAPIIS5 (initiation form), RNAPIIS2P (elongation form), RNAPIIS2P/S5P, and Omni-ATAC. CUTAC for RNAPIIS5P shows the best sensitivity (most peaks at low sampling) and the best signal-to-noise (highest FRiP at all sampling levels). Tagmentation was for 10 min at 37° C. in CUTAC-tag buffer.

FIGS. 17A-17E: Pol2S5p CUTAC maps promoters and functional enhancers adjacent to RNAPII genome-wide: (17A) Heatmaps showing occupancies of Pol2S5p CUTAC, H3K4me2 CUTAC and ATAC-seq signals over promoters in K562 cells, which become sharper for subnucleosomal (1-120 bp) fragments. (17B) Pol2S5p CUTAC, H3K4me2 CUTAC and ATAC-seq signals precisely mark functional enhancers when aligned to STARR-seq peaks. (17C) To evaluate the data quality of Pol2S5p CUTAC, random samples of mapped fragments were drawn, mitochondrial reads were removed and MACS2 was used to call (narrow) peaks. The number of peaks called for each sample (left) is a measure of sensitivity and the fraction of reads in peaks (FRiP, right) is a measure of specificity calculated for each sampling in a doubling series from 50,000 to 6.4 million fragments. For comparison, an ENCODE ATAC-seq sample was used for K562 cells and a published ATAC-seq sample from our lab (GSE128499) was used for H1 cells. Hex samples were tagmented in the presence of 10% 1,6-hexanediol. (17D) Run-on transcription initiates from most sites corresponding to RNAPIIS5P CUTAC peaks, where PRO-seq maps the RNA base at the active site of paused Pol2 (Mahat D B, et al. Base-pair-resolution genome-wide mapping of active RNA polymerases using precision nuclear run-on (PRO-seq). Nat Protoc 2016, 11:1455-1476). Both plus and minus strand PRO-seq datasets downloaded from GEO (GSM3452725) were pooled and aligned over peaks called by MACS2 using 3.2 million RNAPIIS5P CUTAC fragments. (17E) Model for RNAPIIS5P-tethered tagmentation of adjacent accessible DNA, where the Set1 H3K4 methyltransferase di- and tri-methylates nucleosomes near stalled Pol2.

FIGS. 18A-18C: CUT&Tag2for1 simultaneously profiles active and repressive elements. (18A) Schematic describing the CUT&Tag2for1 rationale: A joint CUTAC-CUT&Tag profile is generated using antibodies against Pol2S5p and H3K27me3, and enriched regions are assigned post-hoc based on fragment size. (18B) Genome browser screenshot showing a CUT&Tag2for1 profile in comparison with H3K27me3 CUT&Tag and Pol2S5p-CUTAC for a representative region in K562 cells, along with enriched peaks. CUT&Tag2for1 large and small fragment peaks as assigned by fragment size are shown. (18C) Heatmaps describing H3K27me3 CUT&Tag (red) and RNA Pol2S5p-CUTAC (blue) enrichment at large fragment (left) or small fragment (right) peaks as defined from CUT&Tag2for1 profiles.

FIGS. 19A-19F: Deconvolution of CUT&Tag2for1 using fragment size and feature width: (19A) Schematic of the single-cell CUT&Tag2for1 experimental rationale, in which two cell types are profiled in bulk in parallel and then arrayed on an ICELL8 microfluidic chip for cell-specific barcoding via amplification and mixing before sequencing. (19B) Schematic of the deconvolution approach using a Bayesian model by considering differences in fragment length distributions and feature widths of the two targets. PDF: Probability density function. (19C) Genome browser screenshot showing a CUT&Tag2for1 profile in comparison with H3K27me3 CUT&Tag and Pol2S5p-CUTAC for a representative region in H1 human embryonic stem cells (hESC), along with inferred peaks from single-cell CUT&Tag2for1 data. (19D) Same as (19C) for K562 cells. (19E and 19F) Single antibody and CUT&Tag2for1 data at the inferred Pol2S5p (left) and H3K27me3 peaks (right) for H1 and K562 cells. In 19C-19F, CUT&Tag2for1 data P5K27 represents the pseudo-bulk derived by pooling single-cell data and Pol2S5p and H3K27me3 data is from single antibody data. Results were obtained by pooling cells from two single-cell replicates.

FIGS. 20A-20E: Single-cell CUT&Tag2for1: (20A) UMAPs representing the low dimensional embedding of cells using inferred Pol2S5p peaks (left), colored by the log of the library size of single cells. (20B) Same as (20A), using inferred H3K27me3 peaks. (20C) Same as (20A), using both inferred Pol2S5p and H3K27me3 peaks. (20D) Plot comparing the number of fragments mapping to Pol2S5p and H3K27me3 peaks for H1 and K562 cells (left). UMAP of cells colored by fraction of fragments mapping to H3K27me3 peaks (right). (20E) Heatmap of normalized fragment counts for the top 400 most variable Pol2S5p and H3K27me3 peaks from Principal Component 1. Results are shown for replicate 1.

FIGS. 21A-21C: Feature definition and fragment length separation under CUTAC conditions. (21A) Fragments were mapped to hg19, and 3.2 million fragments were randomly sampled from each dataset and used to make bedgraph tracks. A representative region is shown. To compare peaks with very different signal-to-noise levels, samples were group-autoscaled with ranges indicated to the left of each set of tracks. Pol2S5p CUTAC of K562 cells with linear pre-amplification using only P5 primers for 12 cycles was followed by addition of P7 primers and PCR for various numbers of cycles. (21B) Size distributions were not affected by differences in the number of PCR cycles following linear amplification. (21C) Fragment length distributions for K562 cells (left) and H1 ES cells (right) are shown for linear pre-amplified fragments after tagmentations in 10 mM TAPS+5 mM MgCl₂±1,6-hexanediol plotted as fractions of the total mapped fragments for linear pre-amplified datasets. Tagmentation in 1,6-hexanediol generally results in a smaller fragment distribution, especially conspicuous for H1 cell nucleosomes. The higher recovery of nucleosome-sized fragments from K562 cells than H1 ES cells reflects the much lower abundance of Polycomb domains in H1 cells.

FIGS. 22A-22D: (22A) Plots comparing the fragment size distribution of CUT&TAG2for1 data in bulk and pseudo-bulk, derived from single-cell data for K562 cells. (22B) Same as (22A) for H1-Hesc cells. (22C) Dirichlet priors and inferred posteriors for fragment size distribution for K562 cells. (22D) Same as (22C), for H1-hESC cells.

FIGS. 23A-23E: (23A) UMAPs representing the low dimensional embedding of cells using inferred Pol2S5p peaks (left). UMAP colored by log of the library size of single cells. (23B) Same as (23A), using inferred H3K27me3 peaks. (23C) Same as (23A), using both inferred Pol2S5p and H3K27me3 peaks. (23D) Plots comparing the number of fragments mapping to Pol2S5p and H3K27me3 peaks (left). UMAP of cells colored by fraction of fragments mapping to H3K27me3 peaks. (23E) Heatmap of normalized fragment counts for highly variable Pol2S5p and H3K27me3 peaks. Results are shown for replicate 2.

FIGS. 24A and 24B: (24A) Single antibody and 2for1 data at the overlapping peaks for K562 cells. (24B) Same as (24A), for H1-hESC cells.

### DETAILED DESCRIPTION

This disclosure is based on the inventor's surprising discovery that a modification of the Cleavage Under Targets & Tagmentation (CUT&Tag) method for antibody-tethered in situ tagmentation can identify NDRs genome-wide at regulatory elements adjacent to transcription-associated histone markers in human cells. As described in more detail below, it was demonstrated that reducing the ionic concentration during tagmentation preferentially attracts Tn5 tethered to the transcription-associated histone modifications (e.g., H3K4me2) via a Protein A/G fusion to the nearby NDR, shifting the site of tagmentation from nucleosomes bordering the NDR to the NDR itself. The large majority of transcription-coupled accessible sites correspond to ATAC-seq sites and vice-versa, and lie upstream of paused RNA Polymerase II (RNAPII). This discovery led to anew method, “CUTAC” (Cleavage Under Targeted Accessible Chromatin), that can be conveniently performed in parallel with ordinary CUT&Tag, producing accessible site maps from low cell numbers, including single cells, with signal-to-noise as good as or better than the best ATAC-seq datasets. Furthermore, it is demonstrated that the method can use other markers for chromatin accessibility or NDRs, such as paused RNAPII itself, and more specifically, the serine 5 phosphorylated RNAPII (referred to as RNAPIIS5P). Finally, it is demonstrated that the CUTAC method can be performed simultaneously using multiple, distinct affinity reagents directed to different antigens, including affinity reagents to markers for chromatin accessibility (e.g., H3K4me2 or RNAPIIS5p) and markers for negative regulatory elements (e.g., H3K27me3) to provide maps that address both the active regulome and silencing regulome in, e.g., single cells. These techniques are all amenable for implementation in a variety of analytical platforms.

In accordance with the foregoing, in one aspect the disclosure provides an in situ method for detecting a site of DNA accessibility in the chromatin of a cell (or population of cells) or cell nucleus (or population of cell nuclei). The method comprises contacting a permeabilized cell with a first affinity reagent that specifically binds a nucleosome depleted region (NDR) marker. The first affinity reagent is coupled to at least one transposome that comprises at least one transposase and a transposon. The transposon comprises a first DNA molecule comprising a first transposase recognition site, and a second DNA molecule comprising a second transposase recognition site. The method also comprises activating the at least one transposase under low ionic conditions, such as with the addition of Mg++. When activated, the at least one transposase cleaves the chromatin DNA and integrates the first and second DNA molecules on either side of the imposed break in the chromatin DNA, which is referred to as “tagging” or “tagmenting” the chromatin DNA. With the cleaving and tagging actions of two transposases, a segment of chromatin DNA that contacts the NDR marker or is proximate to the NDR is excised with a tag on either end of the segment. The method then comprises isolating the excised tagged DNA segment, and subjecting it to analysis, such as determining the sequence of the excised tagged DNA segment. The determined sequence can be designated as a site of DNA accessibility, e.g., a site in the chromatin associated with transcription or at least transcription accessibility. The method thereby provides for detecting the site of DNA accessibility in the chromatin of the cell.

The general method is based on the CUT&Tag methodology, which is described in more detail in WO2019060907, incorporated herein by reference in its entirety, but contains key modifications that result in improved detection of DNA accessibility sequences with great sensitivity and consistency. The method incorporates affinity reagent-directed targeting of tagmentation allowing the isolation of chromatin DNA segments associated with targeted regions of the genome, such as transcription-“accessible” regions, e.g., NDRs. The tagmentation can be implemented using a transposase that is activated upon targeting to the desired location on the chromatin and integrated DNA molecules (tags) that provide appropriate functionality for analysis on any appropriate analytic platform (e.g., NGS sequencer).

As indicated above and described below in more detail, it was found that providing low ionic conditions during the tagmentation of the genome allowed specific tagmentation of target sites, e.g., transcription accessible sites or NDRs associated with transcription activity in the genome, with greater precision and higher signal-to-noise than all previous methods, such as ATAC-seq. In this context, the low ionic concentration refers to a low monovalent ionic concentration. Monovalent ions can be supplied by salts with monovalent cations such as Na⁺ Li⁺, Na⁺, etc., or anions such as Cl⁻, and SO4⁻. Often, the salt component of the reaction environment is NaCl, but other sources of monovalent ions are possible. In some embodiments the low ionic conditions are characterized by a monovalent concentration less than about 10 mM. Exemplary low monovalent ionic concentrations include less than about 10 mM, less than about 9 mM, less than about 8 mM, less than about 7 mM, less than about 6 mM, less than about 5 mM, less than about 4 mM, less than about 3 mM, less than about 2 mM, and less than about 1 mM. Exemplary ranges of low monovalent ionic conditions include monovalent concentrations between about 1 mM to about 10 mM, about 2 mM to about 9 mM, about 3 mM to about 8 mM, about 4 mM to about 7 mM, about 5 mM to about 6 mM.

With reference to the prior CUT&Tag protocols (see, e.g., WO2019060907), this low ionic condition can be accomplished with a variety of alterations to the standard protocols. For example, in some embodiments, the low ionic conditions are obtained by diluting liquid conditions of the transposase with a Mg⁺⁺ solution. In other embodiments, the low ionic conditions are obtained by diluting the liquid conditions with TAPS buffer. In illustrative embodiments, reference is made to the exemplary protocol disclosed in Example 2, where a functional dilution of the ionic concentration exposed to the transposase can be accomplished conducting the method until step 21, after which it is held at room temperature and the protocol skips to step 27. Once chilled according to step 28, the method skips to step 34. In other embodiments, the low ionic conditions are obtained by removing liquid supernatant from the transposase and replacing it with a low ionic strength solution. For example, with reference to the exemplary protocol disclosed in Example 2, the method is performed until step 23, upon which the sample is held on ice and the method skips to step 29. Once the sample is chilled in step 30, the method skips to step 34. In yet other embodiments, the low ionic conditions are obtained by conducting a stringent (e.g., 300 mM) wash followed by adding a low ionic strength solution, as described above. For example, with reference to the exemplary protocol disclosed in Example 2, the method is performed until step 26, upon which the method skips to step 31 and then skips to step 33.

The first affinity reagent serves a targeting function to focus the activity of the coupled transposome to an antigen of interest on the chromatin of the cell. As indicated the antigen can be a marker for NDR and, thus, associate with regions of DNA accessibility (i.e., potential assembly of transcriptional machinery for expression of a gene. In such embodiments, the NDR marker can be a histone modification (e.g., methylation, including bi-methylated and tri-methylated states) associated with opening of the chromatin. For example, the NDR marker can be a methylated H3K4, such as H3K4me2 or H3K4me3. It is described below that chromatin accessibility is driven by RNAPII transcription initiation. Thus, in other embodiments, the NDR marker is a paused or initiation form of RNA Polymerase II. The initiation form of RNAPII, which has a serine-5 phosphate on the repeated heptameric C-terminal domain of the largest subunit (referred to as RNAPIIS5P), precisely aligns with chromatin accessibility. The elongation form of RNAPII, which has a serine-2 phosphate on the repeated heptameric C-terminal domain of the largest subunit (referred to as RNAPIIS2P), also precisely aligns with chromatin accessibility. Thus, RNAPII and its post-translational modifications (e.g., the S2P or S5P forms) can serve as a powerful marker that can be targeted by the first affinity reagent of the disclosed method. While the method is described in the context of a first affinity reagent targeting a marker for genome accessibility (e.g., an NDR marker), it will be appreciated that the disclosure encompasses use of first affinity reagent that specifically binds any target of interest on the chromatin DNA. Additional examples are described in more detail below.

The first affinity reagent is coupled to at least one transposome. In some embodiments, the first affinity reagent is coupled to a single transposome. In some embodiments, the first affinity reagent is coupled to a plurality (i.e., two or more) of transposomes. The coupling can occur before, during, or after the first affinity reagent is contacted to the permeabilized cell.

The coupling of the first affinity reagent with the at least one transposase can be direct or indirect. For example, in some embodiments, the first affinity reagent is directly coupled to the at least one transposase. The present disclosure encompasses any coupling or tether structure, whether covalent or non-covalent. In one exemplary embodiment, the first affinity reagent and the transposase are each domains disposed in a single fusion protein. In some embodiments, the first affinity reagent is directly coupled to the at least one transposase by, e.g., biotin/streptavidin type associations.

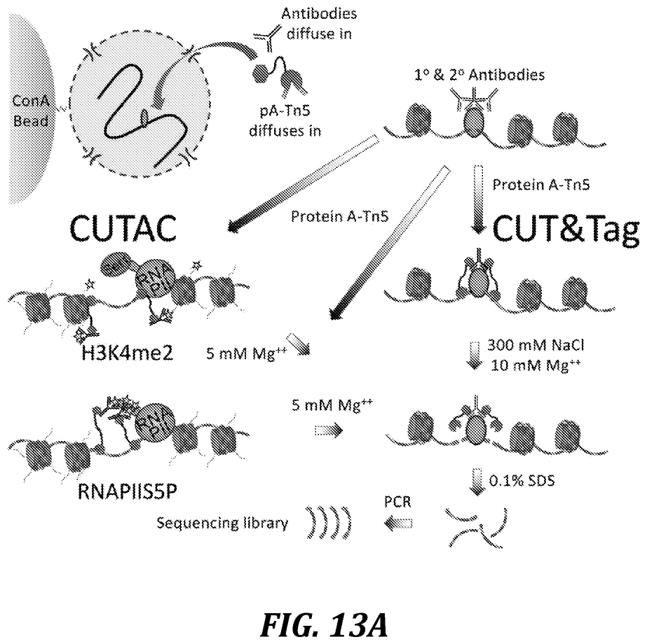

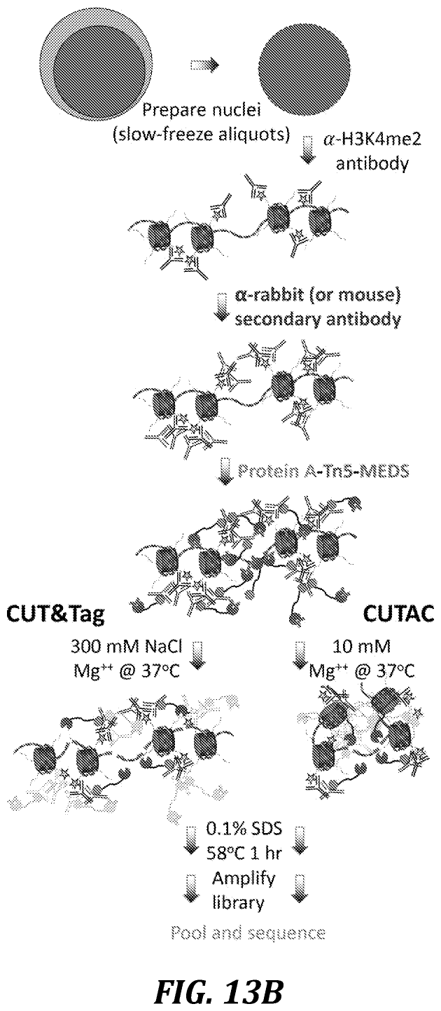

In other embodiments, the first affinity reagent is indirectly coupled to the at least one transposase via at least one intermediary construct. For example, the transposase can be linked to a specific binding agent that specifically binds the first affinity reagent. In other embodiments, the method further comprises contacting the cell with a second affinity reagent that specifically binds the first affinity reagent, and wherein the transposase is linked to a specific binding agent that specifically binds the second affinity reagent. This is similar to a “sandwich ELISA” setup. To illustrate, in one specific and nonlimiting example, the first affinity reagent is a primary antibody that specifically binds to the NDR marker. One or more secondary antibodies specific for, e.g., the constant domain of the primary antibody can be contacted to the bound primary antibody to allow binding. Each of the one or more second antibodies can bind to the first antibody while being also coupled (directly or also indirectly) to at least one transposase. In this fashion an increased number of active transposases can be indirectly targeted to the antigen of the first antibody and the ultimate transposase activity is amplified at any given target site. This concept can be further extended by inclusion of yet additional intermediary affinity reagents. For example, in yet other embodiments, the method further comprises contacting the cell with a second affinity reagent that specifically binds the first affinity reagent and contacting the cell with a third affinity reagent that specifically binds the second affinity reagent. The transposase can be coupled directly to the third affinity reagent or indirectly to the third affinity reagent (e.g., via a specific binding agent that specifically binds the third affinity reagent). Again, the use of additional intermediary affinity reagents (i.e., second affinity reagent, third affinity reagent, etc.) allow for amplification of the arsenal of transposomes that are specifically targeted to the desired marker or antigen on the chromatin. A visual representation of this concept is set forth in FIG. 13B, which shows a schematic representation of a primary antibody (i.e., the first affinity reagent) bound to the H3K4me2 antigen, with a plurality of rabbit (or mouse) secondary antibodies (i.e., second affinity reagent) that binds to the primary antibody. The secondary antibodies each are coupled to the transposome via a binding agent, allowing for multiple transposomes to be targeted to the area of chromatin associated with the target H3K4me2 antigen.

As indicated above, in some embodiments a binding agent can be used to directly couple a transposome to an affinity reagent. Protein A, protein G, protein L, and protein Y are proteins that bind to immunoglobulin proteins and, thus, can serve as exemplary binding agents that link transposomes to immunoglobulin-based affinity reagents. Thus, in some embodiments, the binding agent comprises protein A, or a binding domain thereof, protein G or a binding domain thereof, protein L or a binding domain thereof, protein Y or a binding domain thereof, hybrid domains thereof (e.g., a protein A/G hybrid binding domain), or an additional (e.g., “fourth”) affinity reagent that specifically binds the first affinity reagent, the second affinity reagent and/or the third affinity reagent, which are described above. Exemplary protein A and protein G include the staphylococcal protein A (pA) or to all or part of staphylococcal protein G (pG) or to both pA and pG (pAG). These proteins have indeed different affinities for rabbit and mouse IgG. The disclosure also encompasses immunoglobulin-binding derivatives or fragments of the pA or pG, and even fusions thereof. In some embodiments, the pA moiety contains 2 IgG binding domains of staphylococcal protein A, i.e., amino acids 186 to 327 of Genbank AAA26676 (which is hereby incorporated by reference as available on Sep. 25, 2017). Variants that retain the activity are also contemplated, such as those having a sequence identity of at least 70% 80%, 90%, 95% or even 99% identity to amino acids 186 to 327 of Genbank AAA26676. The disclosure is however not limited to this specific fusion protein.

In some embodiments, the transposome comprises a fusion protein of the transposase and the binding agent. For example, the transposome can comprise a Tn5 transposase domain and protein A or a binding domain thereof, protein G or a binding domain thereof, a protein A/G hybrid binding domain, and the like. Exemplary protein A and protein G domains are described above. Alternatively, the transposase can be linked chemically to the X domain by a bond other than a peptidic bond, or even non-covalently (e.g., with biotin/avidin interactions, etc.)

The affinity reagents (i.e., first, second, third, fourth, etc. affinity reagents) as described above can be any reagent that can specifically bind to its respective target. For example, in some embodiments the first affinity reagent specifically binds to the NDR marker, an exemplary second affinity reagent specifically binds to the first affinity reagent (without negatively affecting the first affinity reagent's ability to bind its respective target), and so on. The first, second, third, and/or fourth affinity reagents can be independently selected from (or comprise) an antibody, an antibody-like molecule, a DARPin, an aptamer, other specifically binding molecule, or a functional antigen-binding domain thereof. In some embodiments, the antibody-like molecule is an antibody fragment and/or antibody derivative. In some embodiments, the antibody-like molecule is a single chain antibody, a bispecific antibody, an Fab fragment, an F(ab)2 fragment, a V_{H}H fragment, a V_{NAR} fragment, or a nanobody. In some embodiments, the single-chain antibody is a single chain variable fragment (scFv), or a single-chain Fab fragment (scFab). Additional description of the affinity reagents encompassed by the disclosure is provided in the definitions section below. In other embodiments, the first affinity reagent can be or comprise a chromatin-binding reagent or functional (i.e., chromatin-binding) fragment thereof.

Chromatin-binding protein reagents include any protein that directly interacts with chromatin, including transcription factors that bind directly to DNA and ‘reader’ proteins/enzymes that interact with and/or modify histones and/or DNA. The chromatin-binding protein can be, without limitation, a transcription factor, a chromatin reader, a histone/DNA modifying enzyme, or a chromatin regulatory complex. Exemplary transcription factors include but are not limited to AAF, abl, ADA2, ADA-NF1, AF-1, AFP1, AhR, AIIN3, ALL-1, alpha-CBF, alpha-CP 1, alpha-CP2a, alpha-CP2b, alphaHo, alphaH2-alphaH3, Alx-4, aMEF-2, AML1, AML1a, AML1b, AML1c, AML1DeltaN, AML2, AML3, AML3a, AML3b, AMY-1L, A-Myb, ANF, AP-1, AP-2alphaA, AP-2alphaB, AP-2beta, AP-2gamma, AP-3 (1), AP-3 (2), AP-4, AP-5, APC, AR, AREB6, Arm, Amt (774 M form), ARP-1, ATBF1-A, ATBF1-B, ATF, ATF-1, ATF-2, ATF-3, ATF-3deltaZIP, ATF-a, ATF-adelta, ATPF1, Barhll, Barhl2, Barx1, Barx2, Bcl-3, BCL-6, BD73, beta-catenin, Bin1, B-Myb, BPT, BP2, brahma, BRCA1, Brn-3a, Brn-3b, Brn-4, BTEB, BTEB2, B-TFIID, C/EBPalpha, C/EBPbeta, C/EBPdelta, CACCbinding factor, Cart-1, CBF (4), CBF (5), CBP, CCAAT-binding factor, CCMT-binding factor, CCF, CCG1, CCK-1a, CCK-1b, CD28RC, cdk2, cdk9, Cdx-1, CDX2, Cdx-4, CFF, ChxlO, CLIMI, CLIM2, CNBP, CoS, COUP, CPl, CPIA, CPIC, CP2, CPBP, CPE binding protein, CREB, CREB-2, CRE-BPl, CRE-BPa, CREMalpha, CRF, Crx, CSBP-1, CTCF, CTF, CTF-1, CTF-2, CTF-3, CTF-5, CTF-7, CUP, CUTL1, Cx, cyclin A, cyclin Tl, cyclin T2, cyclin T2a, cyclin T2b, DAP, DAX1, DB1, DBF4, DBP, DbpA, DbpAv, DbpB, DDB, DDB-1, DDB-2, DEF, deltaCREB, deltaMax, DF-1, DF-2, DF-3, Dlx-1, Dlx-2, Dlx-3, DIx4 (long isoform), Dlx-4 (short isoform, Dlx-5, Dlx-6, DP-1, DP-2, DSIF, DSIF-pl4, DSIF-pl60, DTF, DUX1, DUX2, DUX3, DUX4, E, El 2, E2F, E2F+E4, E2F+p107, E2F-1, E2F-2, E2F-3, E2F-4, E2F-5, E2F-6, E47, E4BP4, E4F, E4F1, E4TF2, EAR2, EBP-80, EC2, EF1, EF-C, EGR1, EGR2, EGR3, EIIaE-A, EIIaE-B, EIIaE-Calpha, EIIaE-Cbeta, EivF, EIf-1, Elk-1, Emx-1, Emx-2, Emx-2, En-1, En-2, ENH-bind. prot, ENKTF-1, EPAS1, epsilonFl, ER, Erg-1, Erg-2, ERR1, ERR2, ETF, Ets-1, Ets-1 deltaVil, Ets-2, Evx-1, F2F, factor 2, Factor name, FBP, f-EBP, FKBP59, FKHL18, FKHRLIP2, Fli-1, Fos, FOXB1, FOXC1, FOXC2, FOXD1, FOXD2, FOXD3, FOXD4, FOXE1, FOXE3, FOXF1, FOXF2, FOXGla, FOXGlb, FOXGlc, FOXH1, FOXI1, FOXJla, FOXJlb, FOXJ2 (long isoform), FOXJ2 (short isoform), FOXJ3, FOXKla, FOXK1b, FOXKTc, FOXL1, FOXMla, FOXMlb, FOXMlc, FOXN1, FOXN2, FOXN3, FOXOla, FOXOlb, FOX02, FOX03a, FOX03b, FOX04, FOXP1, FOXP3, Fra-1, Fra-2, FTF, FTS, G factor, G6 factor, GABP, GABP-alpha, GABP-beta1, GABP-beta2, GADD 153, GAF, gammaCMT, gammaCACl, gammaCAC2, GATA-1, GATA-2, GATA-3, GATA-4, GATA-5, GATA-6, Gbx-1, Gbx-2, GCF, GCMa, GCNS, GF1, GL1, GLI3, GR alpha, GR beta, GRF-1, Gsc, Gscl, GT-IC, GT-IIA, GT-IIBalpha, GT-IIBbeta, H1TF1, H1TF2, H2RIIBP, H4TF-1, H4TF-2, HAND1, HAND2, HB9, HDAC1, HDAC2, HDAC3, hDaxx, heat-induced factor, HEB, HEBl-p67, HEBl-p94, HEF-1 B, HEF-1T, HEF-4C, HEN1, HEN2, Hesxl, Hex, HIF-1, HIF-1alpha, HIF-1beta, HiNF-A, HiNF-B, HINF-C, HINF-D, HiNF-D3, HiNF-E, HiNF-P, HIP1, HIV-EP2, Hlf, HLTF, HLTF (Metl23), HLX, HMBP, HMG I, HMG I(Y), HMG Y, HMGI-C, HNF-IA, HNF-IB, HNF-IC, HNF-3, HNF-3alpha, HNF-3beta, HNF-3gamma, HNF4, HNF-4alpha, HNF4alphal, HNF-4alpha2, HNF-4alpha3, HNF-4alpha4, HNF4gamma, HNF-6alpha, hnRNP K, HOX11, HOXAl, HOXAIO, HOXAIO PL2, HOXA11, HOXA13, HOXA2, HOXA3, HOXA4, HOXA5, HOXA6, HOXA7, HOXA9A, HOXA9B, HOXB-1, HOXB13, HOXB2, HOXB3, HOXB4, HOXBS, HOXB6, HOXA5, HOXB7, HOXB8, HOXB9, HOXC10, HOXC11, HOXC12, HOXC13, HOXC4, HOXC5, HOXC6, HOXC8, HOXC9, HOXD10, HOXD11, HOXD12, HOXD13, HOXD3, HOXD4, HOXD8, HOXD9, Hp55, Hp65, HPX42B, HrpF, HSF, HSF1 (long), HSF1 (short), HSF2, hsp56, Hsp90, IBP-1, ICER-II, ICER-ligamma, ICSBP, Idl, Idl H′, Id2, Id3, Id3/Heir-1, IF1, IgPE-1, IgPE-2, IgPE-3, IkappaB, IkappaB-alpha, IkappaB-beta, IkappaBR, II-1 RF, IL-6 RE-BP, 11-6 RF, INSAF, IPF1, IRF-1, IRF-2, B, IRX2a, Irx-3, Irx-4, ISGF-1, ISGF-3, ISGF3alpha, ISGF-3gamma, 1st-1, ITF, ITF-1, ITF-2, JRF, Jun, JunB, JunD, kappay factor, KBP-1, KER1, KER-1, Koxl, KRF-1, Ku autoantigen, KUP, LBP-1, LBP-la, LBXl, LCR-Fl, LEF-1, LEF-IB, LF-A1, LHX1, LHX2, LHX3a, LHX3b, LHXS, LHX6.1a, LHX6.1b, LIT-1, Lmol, Lmo2, LMX1A, LMX1B, L-Myl (long form), L-Myl (short form), L-My2, LSF, LXRalpha, LyF-1, Lyl-1, M factor, Madl, MASH-1, Maxl, Max2, MAZ, MAZ1, MB67, MBF1, MBF2, MBF3, MBP-1 (1), MBP-1 (2), MBP-2, MDBP, MEF-2, MEF-2B, MEF-2C (433 AA form), MEF-2C (465 AA form), MEF-2C (473 M form), MEF-2C/delta32 (441 AA form), MEF-2D00, MEF-2D0B, MEF-2DA0, MEF-2DAO, MEF-2DAB, MEF-2DA′B, Meis-1, Meis-2a, Meis-2b, Meis-2c, Meis-2d, Meis-2e, Meis3, Meoxl, Meoxla, Meox2, MHox (K-2), Mi, MIF-1, Miz-1, MM-1, MOP3, MR, Msx-1, Msx-2, MTB-Zf, MTF-1, mtTFl, Mxil, Myb, Myc, Myc 1, Myf-3, Myf-4, Myf-5, Myf-6, MyoD, MZF-1, NCI, NC2, NCX, NELF, NER1, Net, NF Ill-a, NF NF-1, NF-IA, NF-1B, NF-1X, NF-4FA, NF-4FB, NF-4FC, NF-A, NF-AB, NFAT-1, NF-AT3, NF-Atc, NF-Atp, NF-Atx, Nf etaA, NF-CLEOa, NF-CLEOb, NFdeltaE3A, NFdeltaE3B, NFdeltaE3C, NFdeltaE4A, NFdeltaE4B, NFdeltaE4C, Nfe, NF-E, NF-E2, NF-E2 p45, NF-E3, NFE-6, NF-Gma, NF-GMb, NF-IL-2A, NF-IL-2B, NF-jun, NF-kappaB, NF-kappaB(-like), NF-kappaBl, NF-kappaB 1, precursor, NF-kappaB2, NF-kappaB2 (p49), NF-kappaB2 precursor, NF-kappaEl, NF-kappaE2, NF-kappaE3, NF-MHCIIA, NF-MHCIIB, NF-muEl, NF-muE2, NF-muE3, NF-S, NF-X, NF-XI, NF-X2, NF-X3, NF-Xc, NF-YA, NF-Zc, NF-Zz, NHP-1, NHP-2, NHP3, NHP4, NKX2-5, NKX2B, NKX2C, NKX2G, NKX3A, NKX3A vl, NKX3A v2, NKX3A v3, NKX3A v4, NKX3B, NKX6A, Nmi, N-Myc, N-Oct-2alpha, N-Oct-2beta, N-Oct-3, N-Oct-4, N-Oct-5a, N-Oct-5b, NP-TCII, NR2E3, NR4A2, Nrfl, Nrf-1, Nrf2, NRF-2betal, NRF-2gammal, NRL, NRSF form 1, NRSF form 2, NTF, 02, OCA-B, Oct-1, Oct-2, Oct-2.1, Oct-2B, Oct-2C, Oct-4A, Oct4B, Oct-5, Oct-6, Octa-factor, octamer-binding factor, oct-B2, oct-B3, Otxl, Otx2, OZF, p107, p130, p28 modulator, p300, p38erg, p45, p49erg,-p53, p55, p55erg, p65delta, p67, Pax-1, Pax-2, Pax-3, Pax-3A, Pax-3B, Pax-4, Pax-5, Pax-6, Pax-6/Pd-5a, Pax-7, Pax-8, Pax-8a, Pax-8b, Pax-8c, Pax-8d, Pax-8e, Pax-8f, Pax-9, Pbx-la, Pbx-lb, Pbx-2, Pbx-3a, Pbx-3b, PC2, PC4, PC5, PEA3, PEBP2alpha, PEBP2beta, Pit-1, PITX1, PITX2, PITX3, PKNOXI, PLZF, PO-B, Pontin52, PPARalpha, PPARbeta, PPARgammal, PPARgamma2, PPUR, PR, PR A, pRb, PRD1-BF1, PRDI-BFc, Prop-1, PSE1, P-TEFb, PTF, PTFalpha, PTFbeta, PTFdelta, PTFgamma, Pu box binding factor, Pu box binding factor (B JA-B), PU.1, PuF, Pur factor, Rl, R2, RAR-alphal, RAR-beta, RAR-beta2, RAR-gamma, RAR-gammal, RBP60, RBP-Jkappa, Rel, RelA, RelB, RFX, RFXl, RFX2, RFX3, RFXS, RF-Y, RORalphal, RORalpha2, RORalpha3, RORbeta, RORgamma, Rox, RPF1, RPGalpha, RREB-1, RSRFC4, RSRFC9, RVF, RXR-alpha, RXR-beta, SAP-la, SAPlb, SF-1, SHOX2a, SHOX2b, SHOXa, SHOXb, SHP, SIII-pl lO, SIII-pl5, SIII-pl8, SIM′, Six-1, Six-2, Six-3, Six-4, Six-5, Six-6, SMAD-1, SMAD-2, SMAD-3, SMAD-4, SMAD-5, SOX-11, SOX-12, Sox-4, Sox-5, SOX-9, Spl, Sp2, Sp3, Sp4, Sph factor, Spi-B, SPIN, SRCAP, SREBP-la, SREBP-lb, SREBP-lc, SREBP-2, SRE-ZBP, SRF, SRY, SRPl, Staf-50, STATlalpha, STATlbeta, STAT2, STAT3, STAT4, STAT6, T3R, T3R-alphal, T3R-alpha2, T3R-beta, TAF(I)110, TAF(I)48, TAF(I)63, TAF(II)100, TAF(II)125, TAF(II)135, TAF(II)170, TAF(II)18, TAF(II)20, TAF(II)250, TAF(II)250Delta, TAF(II)28, TAF(II)30, TAF(II)31, TAF(II)55, TAF(II)70-alpha, TAF(II)70-beta, TAF(II)70-gamma, TAF-I, TAF-II, TAF-L, Tal-1, Tal-lbeta, Tal-2, TAR factor, TBP, TBX1A, TBX1B, TBX2, TBX4, TBXS (long isoform), TBXS (short isoform), TCF, TCF-1, TCF-1A, TCF-1B, TCF-1C, TCF-1D, TCF-1E, TCF-1F, TCF-1G, TCF-2alpha, TCF-3, TCF-4, TCF-4(K), TCF-4B, TCF-4E, TCFbetal, TEF-1, TEF-2, tel, TFE3, TFEB, TFIIA, TFIIA-alpha/beta precursor, TFIIA-alpha/beta precursor, TFIIA-gamma, TFIIB, TFIID, TFIIE, TFIIE-alpha, TFIIE-beta, TFIIF, TFIIF-alpha, TFIIF-beta, TFIIH, TFIIH*, TFIIH-CAK, TFIIH-cyclin H, TFIIH-ERCC2/CAK, TFIIH-MAT1, TFIIH-M015, TFIIH-p34, TFIIH-p44, TFIIH-p62, TFIIH-p80, TFIIH-p90, TFII-I, Tf-LFl, Tf-LF2, TGIF, TGIF2, TGT3, THRAl, TIF2, TLE1, TLX3, TMF, TR2, TR2-11, TR2-9, TR3, TR4, TRAP, TREB-1, TREB-2, TREB-3, TREFl, TREF2, TRF (2), TTF-1, TXRE BP, TxREF, UBF, UBP-1, UEF-1, UEF-2, UEF-3, UEF-4, USF1, USF2, USF2b, Vav, Vax-2, VDR, vHNF-lA, vHNF-lB, vHNF-lC, VITF, WSTF, WTT, WT1I, WT1 I-KTS, WT1 I-del2, WT1-KTS, WT1-del2, X2BP, XBP-1, XW-V, XX, YAF2, YB-1, YEBP, YYT, ZEB, ZF1, ZF2, ZFX, ZHX1, ZIC2, ZID, ZNF 174, amongst others. Examples of transcription factors also include, without limitation, those listed at: en.wikipedia.org/wiki/List_of_human_transcription_factors, incorporated by reference herein in its entirety. Examples of readers include, without limitation, BRD4, YEATS2, and PWWP. Examples of histone/DNA modifying enzymes include, without limitation, NSD2, JMJD2A, CARM1, MLL1, DOT1L, EZH2, and DNMT3A/B. Examples of chromatin regulatory complexes include, without limitation, RNA Polymerase II, SMARCA2, and ACF.

Nucleosome deficient regions (NDRs) can be characterized as “holes” in the chromatin landscape. However, these holes can still often present a sufficiently restricted structure that prevents full or easy access to multiple transposomes to efficiently activate, tagment, and excise target regions of the accessible genome. Accordingly, in some embodiments the method further comprises contacting the permeabilized cell with a polar compound prior to or during the step of activating the transposase under low ionic conditions. The polar compounds can further disrupt some of the intra-chromatin interactions to allow a loosening of the structure, i.e., a loosening of the hole to allow multiple transposomes to access the chromatin DNA and tagment in their respective ends within the region. Illustrative examples of the polar compound include 1,6-hexanediol and N,N-dimethylformamide, although the disclosure encompasses other polar compounds identifiable by persons of ordinary skill in the art.

As indicated above, transposase activities of two transposons are required to tagment and excise chromatin DNA associated with the targeted antigen (e.g., the NDR marker). In some embodiments, the permeabilized cell is contacted with a plurality of first affinity reagents (i.e., affinity reagents that specifically bind the desired NDR marker, which may reside at several locations in the chromatin), each of the first affinity reagents being coupled (directly or indirectly) to at least one transposome, as described above. Alternatively or additionally, incorporation of additional affinity reagents (i.e., second affinity reagents, third affinity reagents, etc.) that serve as intermediary constructs can amplify the number of transposomes that are associated with a single first affinity reagent. This configuration targets the plurality of transposomes to the single target marker. By employing a plurality of transposomes with transposase activities, multiple cleavage events in the chromatin DNA are implemented during the activating step in a restricted region containing or proximate to the target marker. This allows for tagmentation and excision of chromatin DNA segments associated with (e.g., bound to or proximate to) the target marker and which are tagged at either end.

The excised tagged DNA segment(s) is/are isolated. The term “isolated” refers to the component (e.g., tagged DNA segment) being substantially separated or purified away from other components of the reaction or cell. For example, the tagged DNA segment can be isolated from other components of the cell, including extra-chromatin DNA and RNA, proteins and organelles. Isolation can be performed using known techniques amenable to nucleic acid analysis. It will be understood that the term “isolated” does not imply that the biological component is “purified”, i.e., free of all trace contamination, but rather can include nucleic acid molecules that are at least 50% isolated, such as at least 75%, 80%, 90%, 95%, 98%, 99%, or even 100% isolated.

The isolated tagged DNA segment that is excised from the chromatin can be subject to further analysis, such as size characterization, fingerprinting, or full sequencing. In some embodiments, the excised DNA is subjected to salt fractionation to facilitate the further analysis. Some of these analyses can be facilitated by the DNA sequencing tags incorporated onto the ends of the DNA segment by the multiple (i.e., two) transposomes that cleaved the DNA and integrated their respective first and second DNA molecules on either side of the breakpoints. Thus, the resulting excised DNA fragment is tagged at one end with a tag (first or second DNA molecule from one transposome) and is tagged at the other end with a tag (first or second DNA molecule from another transposome). Thus, when referring to a single excised tagged DNA segment, these tags at either end can be referred to a first and second DNA molecule tag, but which do not correspond to the first and second DNA molecules integrated by a single transposome. Instead, the tags on the excised DNA segment correspond with either the first or second DNA molecule of a one transposome and either the first or second DNA molecule of another transposome, respectively, in any combination. To promote the indicated analyses, the first and/or second DNA molecule of the transposome(s) can further comprise a barcode. The barcode(s) can serve to include identifying tag information to allow tracking and identification of the originating cell, batch, position, or other relevant information, to facilitate analysis in the various analytic platforms. Additionally or alternatively, the first and/or second DNA molecule of the transposome(s) can comprise a sequencing adaptor sequence. In some embodiments, the first and/or second DNA molecule further comprises a universal priming site. These domains in the first and/or second DNA molecule of the transposome(s) will result in isolated excised tagged DNA segments that can be amplified and subjected to any appropriate DNA sequencing technology. For example, sequencing can be performed using automated Sanger sequencing (AB 13730x1 genome analyzer), pyrosequencing on a solid support (454 sequencing, Roche), sequencing-by-synthesis with reversible terminations (ILLUMINA® Genome Analyzer), sequencing-by-ligation (ABI SOLiD®) or sequencing-by-synthesis with virtual terminators (HELISCOPE®). Other next generation sequencing techniques for use with the disclosed methods include, Massively parallel signature sequencing (MPSS), Polony sequencing, Ion Torrent semiconductor sequencing, DNA nanoball sequencing, Heliscope single molecule sequencing, Single molecule real time (SMRT) sequencing, and Nanopore DNA sequencing. Thus, in some embodiments, the excised chromatin DNA is analyzed, for example by determining the nucleotide sequence. In some examples, the nucleotide sequence is determined using sequencing or hybridization techniques with or without amplification.

In some embodiments, the first affinity reagent is coupled (e.g., indirectly) to a plurality of transposomes, the plurality of transposomes comprising at least two transposomes that differ in at least the first and second DNA sequences that they integrate into the chromatin DNA upon activation. Alternatively, the at least two transposomes can contain first and second DNA sequences that are identical from transposome to transposome.

The transposase in the transposome can be any functional protein or domain with transposase activity to appropriately cleave and integrate the first and second DNA molecules into the DNA at either side of the breakpoint. The transposase is preferably inducible, such that activity can be controlled.

The disclosed methods can use any transposase. Exemplary embodiments can include the use of a hyperactive Tn5 transposase and a Tn5-type transposase recognition site (Goryshin and Reznikoff, J. Biol. Chem., 273:7367 (1998)), or MuA transposase and a Mu transposase recognition site comprising R1 and R2 end sequences (Mizuuchi, K., Cell, 35: 785, 1983; Savilahti, H, et al, EMBO J., 14: 4893, 1995). A transposase recognition site forms a complex with a hyperactive Tn5 transposase (e.g., EZ-Tn5™ Transposase). More examples of transposition systems that can be used with certain embodiments provided herein include Staphylococcus aureus Tn552 (Colegio et al, J. Bacteriol, 183: 2384-8, 2001; Kirby C et al, Mol. Microbiol, 43: 173-86, 2002), Tyl (Devine & Boeke, Nucleic Acids Res., 22: 3765-72, 1994 and International Publication WO 95/23875), Transposon Tn7 (Craig, N L, Science. 271: 1512, 1996; Craig, N L, Review in: Curr Top Microbiol Immunol, 204:27-48, 1996), Tn/O and IS 10 (Kleckner N, et al, Curr Top Microbiol Immunol, 204:49-82, 1996), Mariner transposase (Lampe D J, et al, EMBO J., 15: 5470-9, 1996), Tel (Plasterk R H, Curr. Topics Microbiol. Immunol, 204: 125-43, 1996), P Element (Gloor, G B, Methods Mol. Biol, 260: 97-1 14, 2004), Tn3 (Ichikawa & Ohtsubo, J Biol. Chem. 265: 18829-32, 1990), bacterial insertion sequences (Ohtsubo & Sekine, Curr. Top. Microbiol. Immunol. 204: 1-26, 1996), retroviruses (Brown, et al, Proc Natl Acad Sci USA, 86:2525-9, 1989), and retrotransposon of yeast (Boeke & Corces, Annu Rev Microbiol. 43:403-34, 1989). More examples include IS5, TnlO, Tn903, IS91 1, and engineered versions of transposase family enzymes (Zhang et al, (2009) PLoS Genet. 5:e1000689. Epub 2009 Oct. 16; Wilson C. et al (2007) J. Microbiol. Methods 71:332-5) and those described in U.S. Pat. Nos. 5,925,545; 5,965,443; 6,437,109; 6,159,736; 6,406,896; 7,083,980; 7,316,903; 7,608,434; 6,294,385; 7,067,644, 7,527,966; and International Patent Publication No. WO2012103545, all of which are specifically incorporated herein by reference in their entirety. In some embodiments, the transposase is a Tn5 transposase or a hyperactive mutant thereof, or functional domain thereof. In some embodiments, the transposase is a Mu transposon, or functional domain thereof. In yet other embodiments, the at least one transposase comprises an IS5 or an IS91 transposase, or a functional domain thereof. In some embodiments, the first affinity reagent is coupled (e.g., indirectly) to a plurality of transposomes, the plurality of transposomes comprising at least two different transposases.

The transposase can be activated using an exogenous activator. For example, activating the transposase, e.g., Tn5, under low ionic conditions can comprise contacting the transposase with a sufficient amount of Mg⁺⁺ (such as in the salt form of MgCl₂ or MgSO₄). Exemplary concentrations sufficient to activate the Tn5 transposase (or an active domain thereof) are from about 0.1 mM Mg⁺⁺ to about 10 mM Mg⁺⁺, such as about 0.5 mM Mg⁺⁺ to about 8 mM Mg⁺⁺, about 0.75 mM Mg⁺⁺ to about 7 mM Mg⁺⁺, about 1 mM Mg⁺⁺ to about 6 mM Mg⁺⁺, and about 2 mM Mg⁺⁺ to about 5 mM Mg⁺⁺. In some embodiments, the concentration of Mg⁺⁺ sufficient to activate the Tn5 transposase is about 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 8.5 mM, or 10 mM.

The method is generally presented for detecting DNA accessibility in chromatin of the cell. In this context, the cell can be any cell-type of interest, without limitation, except that it contains a genome with chromatin structure, e.g., eukaryotic cells. Exemplary cells include animal (e.g., mammalian, e.g., mouse, rat, human, etc., insect, etc.) and plant cells. It will be appreciated that the disclosure also encompasses other applications of the method to detect other features present in a genome that are not dependent on chromatin structure but are associated with other defined antigens on the DNA (e.g., transcription factors, etc.). Thus, in some such embodiments, the cell could also be a prokaryotic cell, e.g., bacterial cell.

Further, while this aspect of the disclosure is generally described in terms of a cell, the method can also be performed on a cell nucleus (or population of cell nuclei). By using intact cells or nuclei the disclosed methods have the advantage over ChIP methods of looking at native chromatin structure, which otherwise might be altered by fragmentation and other processing steps.

The cell and/or the nucleus of the cell can be permeabilized prior to the start of the method, or the method can include a step of actively permeabilizing the cell or cell nucleus. The cell and/or the nucleus of the cell can be permeabilized by contacting the cell and/or nucleus with a permeabilizing agent, such as with a detergent, for example Triton and/or NP-40 or another agent, such as digitonin. Digitonin partitions into membranes and extracts cholesterol. Membranes that lack cholesterol are minimally impacted by digitonin. Nuclear envelopes are relatively devoid of cholesterol compared to plasma membranes. As such, treatment of cells with digitonin represents a robust method for permeabilizing cells without compromising nuclear integrity. Exemplary protocols described below use digitonin, but it is possible that individual experimental situations call for generating intact nuclei by other means, and such nuclei can be prepared by any suitable method.

A powerful advantage of the disclosed method is the sensitivity allows analysis of the chromatin (or other genomic characteristics) of a single cell, although the method can also be performed in a batch context. For example, the method can be performed for a plurality of cells, wherein the method further comprises mapping the determined sequences of one or more excised tagged DNA segments to a genome representing the plurality of the cells. This can be referred to as a consensus genome. Typically, the cells in such a batch would be derived from the same species or individual. At a single cell level, the one or more determined sequences obtained from the particular cell can be mapped to the genome of the cell. Further, even single cell implementation of the method can be massively scaled up to address a large number of cells in parallel. The method is readily adaptable to a variety of analytic platforms, in part, because there is no restriction to the presentation of the permeabilized cell. For example, the cell can be situation free in solution or can be immobilized on a solid support or surface, such as a bead, wall of a microtiter plate, on a two dimensional array (e.g., in a tissue slice) or in a three dimensional matrix.

As indicated, a variety of analytic platforms can be adapted to incorporate the present method to facilitate performing the analysis to scale and/or to address specific analytic contexts. Such analytic platforms incorporate a variety of cell processing and handling contexts, such as in microfluidic, droplets, well and nano-well arrays, three-dimensional tissue or cell arrays, and the like. These platforms provide contexts in which the cells can be handled and manipulated for application of the method. For example, the present method can be applied in spatial transcriptomic approaches, where cells existing in a defined three dimensional space (e.g., a tissue slice or fixed on an array or matrix) are analyzed at a sub-batch or single cell level. For example, NanostringGeoMx (NanoString Technologies, Seattle, WA) provides for gene expression profiling with spatial resolution of immunohistochemistry. Visium Spatial Gene Expression (10× Genomics, Pleasanton, CA) is a next-generation molecular profiling platform used to classify tissue based on total mRNA. The platform uses spatially barcoded mRNA-binding oligonucleotides to imprint unique barcodes across a three dimensional space. While the resolution of the platform is improving, it is advancing toward single-cell resolution. Other approaches employ deterministic barcoding in tissue preparations for spatial omics sequencing (DBiT). Generally, a tissue slice is placed on a slide and the reactions are carried out on the slide. A microfluidic chip is placed on top for X-axis barcoding followed by a second chip for Y-axis barcoding, both at ˜20 μm resolution, resulting in single-cell resolution at each X-Y intersection. One platform is developed by AtlasXomics (New Haven, CT). Other exemplary DBiT applications encompassed by the disclosure are described in Liu, Y., et al. (2020) High-Spatial-Resolution Multi-Omics Sequencing via Deterministic Barcoding in Tissue, 183(6), 1665-1681; Deng, Y., et al. (2021) Spatial-ATAC-seq: spatially resolved chromatin accessibility profiling of tissues at genome scale and cellular level, bioRxiv 2021.06.06.447244; and Deng, Y., et al. Spatial Epigenome Sequencing at Tissue Scale and Cellular Level, bioRxiv 2021.03.11.434985, each of which is specifically incorporated herein by reference in its entirety. In other approaches, barcodes are first arrayed on a slide, and a tissue section is placed on top. While such approaches are implemented mostly for RNA-seq, they can be adapted for the tagmentation approach of the present method. An exemplary such method is random splint ligation (e.g., Maguire, Gregory, et al., (2020) A low-bias and sensitive small RNA library preparation method using randomized splint ligation, Nucleic Acids Research, 4814, page e80), which can be adapted for spatial profiling methods. In Slide-seqV2, a slide is coated with a monolayer of barcoded polystyrene beads to provide a high-resolution array that is used to capture tissue RNA (see Stickels, R. R., et al. (2021) Highly sensitive spatial transcriptomics at near-cellular resolution with Slide-seqV2, Nat Biotechnol 39, 313-319). In Seq-Scope, a pre-barcoded array is bridge-amplified and sequenced as an Illumina flow cell to get the barcode. After barcodes are established spatially, a tissue slice DNA is captured onto the array and re-sequenced to get the cellular RNA sequence (Cho, C.-S., et al. (2021) Microscopic examination of spatial transcriptome using Seq-Scope, Cell 184(13), 3559-3572). Similar technologies include PIXEL-seq (Fu, X, et al. Continuous Polony Gels for Tissue Mapping with High Resolution and RNA Capture Efficiency, bioRxiv 2021.03.17.435795) and Stereo-seq, which uses a patterned oligo array that employs the BGI nanoball sequencing platform (Chen, A., et al. (2021) Large field of view-spatially resolved transcriptomics at nanoscale resolution, bioRxiv 2021.01.17.427004). In sci-Space, a tissue section is placed on a slide for arrayed barcoded oligonucleotide update and imaging. The nuclei are then extracted and processed for RNA-seq (Srivatsan, S. R., et al., Embryo-scale, single-cell spatial transcriptomics, Science, 373(6550), 111-117). There has also been rapid progress on several imaging-based spatial transcriptomics approaches, either by direct priming and sequencing of RNAs in situ or by FISH, using combinations of fluorophores incorporated during RNA-seq or successively hybridized. All of these platforms can be adapted for processing of cells or nuclei of the present disclosure to impose affinity reagent-targeted unique tagmentation and excision of the chromatin DNA segments that are then tracked and analyzed for chromatin accessibility (or another antigen-specific trait).

Yet other platforms amenable for adaptation to incorporate the present methods focus on providing single-cell or near single-cell omics analysis, regardless of tissue or spatial origin. For example, to facilitate such single-cell analysis, each cell of a plurality of cells is processed to receive a tag with a cell-specific barcode or a cell-specific combination of barcodes. In the context of the present method, these barcodes can be implemented in the various first and second DNA sequences that are implemented by the transposases. The analytic platforms provide an organizational context to allow application of unique barcodes from cell to cell. In combinatorial indexing, for example, at least two levels of barcoding are applied to cells in a pool, where each level of barcoding is achieved by distributing cells from a pool into segregated wells, each well containing reagents comprising a unique barcode. The cells are processed appropriately and re-pooled and redistributed to receive the second or subsequent barcode. The nuclei are only processed after all the barcoding has occurred.

Calibration of the present method can be performed to inform the sensitivity and accuracy of results. This can be implemented by spike-in of additional DNA to serve as a template with predetermined, detectable sequence. The spike in DNA can be applied to the permeabilized cell(s) prior to or simultaneous with the contacting with the first affinity reagent. The spike-in DNA can be any exogenous DNA, exogenous chromatin, or recombinant nucleosome structures. For example, in one embodiment a fraction of the plurality of transposomes can comprise a known amount of spike-in DNA, which is modified and processed in a manner similar to the endogenous chromatin DNA of the cell. As the amount if spike-in DNA is known, the signal obtained from the spike-in DNA can be used to calibrate the overall output of the method.

In other aspects, the disclosed CUTAC method, including all of the embodiments as described above, can be performed in association with (e.g., in parallel with) a CUT&Tag or CUT&Run protocol for parallel analysis. For example, as disclosed in more detail in Example 1, the CUTAC method can be performed with a more traditional CUT&Tag protocol to provide CUT&Tag maps of any selected antigen that can overlay with the DNA accessibility map. Moreover, the two methods can be performed together in the same workflow. Example 2 provides an exemplary step-by-step protocol for performing the CUT&Tag-direct method with the CUTAC method.

In yet another aspect, the disclosed CUTAC method can be performed with multiple affinity reagents simultaneously, each of which serves to target tagmentation (and downstream analysis) to different antigens in the same chromatin DNA of the same cell. The different antigens do not have to be associated with the same types of chromatin region, such as open or accessible chromatin DNA. For example, one affinity reagent is used to target areas of chromatin accessibility (e.g., NDR markers) and another affinity reagent is used to target the markers of negative regulatory elements. The result is a combined map that indicates positive and negative regulomes (i.e., active transcription activity and repression of transcription activity) in the same cell. In the terminology used in the above-described CUTAC method, the first affinity reagent specifically binds an NDR marker, where the other affinity reagent (referred to now as the fifth affinity reagent) specifically binds a repressive regulatory element marker. An exemplary implementation of this combined approach is described in Example 4, which refers to the method as CUT&Tag2for1.

The first affinity reagent and the fifth affinity reagent are contacted to the permeabilized cell (or population of cells) or cell nucleus (or population of nuclei). Like the first affinity reagent, described above, the fifth affinity reagent is coupled (directly or indirectly) to at least one transposome. The transposome coupled to the fifth affinity reagent comprises at least one transposase and a transposon. The transposon comprises a first DNA molecule comprising a first transposase recognition site and a second DNA molecule comprising a second transposase recognition site.

The at least one transposases coupled to the first affinity reagent and the fifth affinity reagent are activated together under low ionic conditions, thereby resulting in cleaving and tagging chromatin DNA with the respective first and second DNA molecules. This activity results in excision of tagged DNA segment(s) associated with the repressive regulatory element marker in addition to excision of tagged DNA segment(s) associated with the NDR marker.

Both sets of excised tagged DNA segments, i.e., those associated with the repressive regulatory element marker and those associated with the NDR marker, are isolated. The method further comprises determining the nucleotide sequence of the excised tagged DNA segments. The sequences associated with the respective markers are deconvoluted, i.e., determined to be associated with the regulatory element marker or the NDR marker to assess, detect, map, and otherwise analyze active and repressive regulomes in the same cell.

Embodiments of the various elements (e.g., coupling arrangement of the affinity reagents and transposomes, transposome/transposase elements, tagmentation elements, isolation and downstream processing and analysis, analytic platforms, and the like) are not repeated in detail here, but rather are described above in the CUTAC discussion and are similarly incorporated in the present aspect.

In some embodiments, the repressive regulatory element marker is a methylated histone, such as methylated, H3K27. In some embodiments, the methylated H3K27 is bi-methylated (H3K27Me2) or tri-methylated (H3K27me3).

In some embodiments, a plurality of sequences is determined from a plurality of excised tagged DNA segments associated with the NDR marker and a plurality of excised tagged DNA segments associated with the repressive regulatory marker. The sequences are generated using any appropriate platform from DNA segments obtained in the same reaction. The sequences are deconvoluted based on aspects of the obtained sequences. For example, a deconvolution algorithm can be applied to the sequences that differentiates the NDR-associated sequences from the negative regulatory element-associated sequences based on different tagmentation densities and/or different fragment sizes associated with the NDR marker and the repressive regulatory marker. An illustrative application of such a deconvolution method is described in more detail in Example 4.

The disclosure also encompasses methods of preparing a library of excised chromatin DNA that is amenable to sequencing on any desired platform. The method comprises the steps described herein above.

In another aspect, the disclosure provides a kit of reagents, and optionally instructions, to facilitate performance of the methods described herein. The kit can comprise one or more of the first affinity reagent, the second affinity reagent, the third affinity reagent, the fourth affinity reagent, the fifth affinity reagent, the transposase (e.g., comprising a transposase domains such as a Tn5 domain), the specific binding agent (e.g., protein A or protein G, or domains thereof, or a hybrid domain thereof), the polar compound, the solid surface (e.g., bead or microtiter plate), the Mg++ solution, a low ionic strength solution, the stringent wash solution, buffers, and other reagents to facilitate performance of a method as described herein, in any combination. These reagents are described in more detail above and all embodiments thereof are encompassed by this aspect and are not repeated here in detail. The kit can optionally include written indicia directing the performance the method as described herein. In some embodiments, the transposase and the specific binding reagent can be included in the same fusion protein construct, as described above. In some embodiments, the kit comprises reagents described below permitting the dual performance of a CUT&Tag protocol and a CUTAC protocol. For example, the kit can comprise a high ionic solution and a low ionic solution to provide high ionic conditions and ionic conditions for transposase activity in parallel containers. For clarity, the optional inclusion of the fifth affinity reagent, which is described above in the context of an additional targeting affinity reagent, facilitates the performance of the CUT&Tag2for1 method, described herein above and in Example 4.

### Additional Definitions

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. For the purposes of the description, a phrase in the form “A/B” or in the form “A and/or B” means (A), (B), or (A and B). For the purposes of the description, a phrase in the form “at least one of A, B, and C” means (A), (B), (C), (A and B), (A and C), (B and C), or (A, B and C). For the purposes of the description, a phrase in the form “(A)B” means (B) or (AB) that is, A is an optional element. Additionally, the words “herein,” “above,” and “below,” and words of similar import, when used in this application, shall refer to this application as a whole and not to any particular portions of the application. The word “about” indicates a number within range of minor variation above or below the stated reference number. For example, in some embodiments “about” can refer to a number within a range of 10%, 9%, 8%, 7%, 6%, 5%, 4%, 3%, 2%, or 1% above or below the indicated reference number.

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art. Additionally, definitions of common terms in molecular biology can be found in Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); and Robert A. Meyers (ed.), Molecular Biology and Biotechnology: a Comprehensive Desk Reference, published by VCH Publishers, Inc., 1995 (ISBN 9780471185710). In case of conflict, the terms in the specification will control.

To facilitate review of the various embodiments of this disclosure, the following explanations of specific terms are provided.

As indicated above, certain embodiments comprise one or more affinity reagents that serve to bind markers associated with DNA accessibility (e.g., nucleosome depleted region (NDR) markers, e.g., transcription-associated histone modification or RNAPIIS5P), to bind markers associated with a negative repressive regulatory element marker (e.g., histone modifications), or bind other affinity reagents or antigens. An affinity reagent is a molecule that can specifically bind to a desired antigen.

The term “specifically binds” refers to, with respect to an antigen, the preferential association of an affinity reagent, in whole or part, with a specific antigen, such as a specific protein bound to chromatin DNA (e.g., a transcription factor, RNAPIIS5P) or modified histone, etc. A specific binding affinity agent binds substantially only to a defined target, such as a specific chromatin associated factor or marker. It is recognized that a minor degree of non-specific interaction may occur between a molecule, such as a specific affinity reagent, and a non-target antigen. Nevertheless, specific binding can be distinguished as mediated through specific recognition of the antigen. Specific binding typically results in greater than 2-fold, such as greater than 5-fold, greater than 10-fold, or greater than 100-fold increase in amount of bound affinity reagent (per unit time) to a target antigen, such as compared to a non-target antigen. A variety of immunoassay formats are appropriate for selecting affinity reagent specifically reactive with a particular antigen. For example, solid-phase ELISA immunoassays are routinely used to select antibodies specifically immunoreactive with a protein. See Harlow & Lane, Antibodies, A Laboratory Manual, Cold Spring Harbor Publications, New York (1988), for a description of immunoassay formats and conditions that can be used to determine specific reactivity.

In some embodiments, the indicated affinity reagent can be an antibody or an antibody-like molecule.

An “antibody” is a polypeptide ligand that includes at least a light chain or heavy chain immunoglobulin variable region and specifically binds an epitope of an antigen, such as a chromatin associated marker or another affinity reagent. The term “antibody” encompasses antibodies, derived from any antibody-producing mammal (e.g., mouse, rat, rabbit, and primate including human), that specifically bind to an antigen of interest (e.g., a chromatin associated marker or another affinity reagent). Exemplary antibody types include multi-specific antibodies (e.g., bispecific antibodies), humanized antibodies, murine antibodies, chimeric, mouse-human, mouse-primate, primate-human monoclonal antibodies, and anti-idiotype antibodies.

Canonical antibodies can be composed of a heavy and a light chain, each of which has a variable region, termed the variable heavy (VH) region and the variable light (VL) region. Together, the VH region and the VL region are responsible for binding the antigen recognized by the antibody. The term “antibody-like molecule” includes functional fragments of intact antibody molecules, molecules that comprise portions of an antibody, or modified antibody molecules, or derivatives of antibody molecules. Typically, antibody-like molecules retain specific binding functionality, such as by retention of, e.g., with a functional antigen-binding domain of an intact antibody molecule. Preferably antibody fragments include the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagent does not have to be naturally occurring or naturally derived, but can be further modified to, e.g., reduce the size of the domain or modify affinity for the antigen as necessary. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Once a monoclonal antibody is identified for inclusion within the bi-specific molecule, the encoding gene for the relevant binding domains can be cloned into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phosphodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/target binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

Designed ankyrin repeat proteins (DARPins) are engineered antibody mimetic proteins that can have highly specific and high affinity target antigen binding. DARPins are typically based on natural ankyrin repeat proteins and comprise at least three repeat motifs. Repetitive structural units (motifs) form a stable protein domain with a large potential target interaction surface. Typically, DARPins comprise four or five repeats, of which the first (N-capping repeat) and last (C-capping repeat) serve to shield the hydrophobic protein core from the aqueous environment. DARPins often correspond to the average size of natural ankyrin repeat protein domains. DARPins can be screened and engineered starting from encoding libraries of randomized variations. Once desired antigen binding characteristics are discovered, the encoding DNA can be obtained. Library screening and use can incorporate ribosome display or phage display.

DNA sequencing refers to the process of determining the nucleotide order of a given DNA molecule. Generally, the sequencing can be performed using automated Sanger sequencing (e.g., using AB 13730x1 genome analyzer), pyrosequencing on a solid support (e.g., using 454 sequencing, Roche), sequencing-by-synthesis with reversible terminations (e.g., using ILLUMINA® Genome Analyzer), sequencing-by-ligation (e.g., using ABI SOLiD®) or sequencing-by-synthesis with virtual terminators (e.g., using HELISCOPE®) other next generation sequencing techniques for use with the disclosed methods include, Massively parallel signature sequencing (MPSS), Polony sequencing, Ion Torrent semiconductor sequencing, DNA nanoball sequencing, Heliscope single molecule sequencing, Single molecule real time (SMRT) sequencing, and Nanopore DNA sequencing

The term “nucleic acid” (molecule or sequence) refers to a deoxyribonucleotide or ribonucleotide polymer including without limitation, cDNA, mRNA, genomic DNA, and synthetic (such as chemically synthesized) DNA or RNA or hybrids thereof. The nucleic acid can be double-stranded (ds) or single-stranded (ss). Where single-stranded, the nucleic acid can be the sense strand or the antisense strand. Nucleic acids can include natural nucleotides (such as A, T/U, C, and G), and can also include analogs of natural nucleotides, such as labeled nucleotides. Some examples of nucleic acids include the probes disclosed herein. The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U). Nucleotides include those nucleotides containing modified bases, modified sugar moieties, and modified phosphate backbones, for example as described in U.S. Pat. No. 5,866,336 to Nazarenko et al. Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N-6-sopentenyladenine, 1-methylguanine, 1-methylinosine, 2,2-dimethylguanine, 2-methyladenine, 2-methylguanine, 3-methylcytosine, 5-methyl cytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxy acetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, 2,6-diaminopurine and biotinylated analogs, amongst others. Examples of modified sugar moieties which may be used to modify nucleotides at any position on its structure include, but are not limited to arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

The terms peptide/protein/polypeptide refer to a polymer of amino acids and/or amino acid analogs that are joined by peptide bonds or peptide bond mimetics. The twenty naturally occurring amino acids and their single-letter and three-letter designations known in the art.

Sequence identity and similarity between multiple nucleic acid or polypeptide sequences can be determined. Sequence identity can be measured in terms of percentage identity; the higher the percentage, the more identical the sequences are. Homologs or orthologs of nucleic acid or amino acid sequences possess a relatively high degree of sequence identity/similarity when aligned using standard methods.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5: 151-3, 1989; Corpet et al, Nuc. Acids Res. 16: 10881-90, 1988; Huang et al. Computer Appls. in the Biosciences 8, 155-65, 1992; and Pearson et al, Meth. Mol. Bio. 24:307-31, 1994. Altschul et al, J. Mol. Biol. 215:403-10, 1990, presents a detailed consideration of sequence alignment methods and homology calculations.

The NCBI Basic Local Alignment Search Tool (BLAST) (Altschul et al, J. Mol. Biol. 215:403-10, 1990) is available from several sources, including the National Center for Biological Information (NCBI, National Library of Medicine, Building 38 A, Room 8N805, Bethesda, Md. 20894) and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1554 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (i.e., 15±20*100=75) The term “transposome” refers to a transposase-transposon complex. A conventional way for transposon mutagenesis usually places the transposase on the plasmid. In some such systems, the transposase can form a functional complex with a transposon recognition site that is capable of catalyzing a transposition reaction. The transposase or integrase may bind to the transposase recognition site and insert the transposase recognition site into a target nucleic acid in a process sometimes termed “tagmentation”.

The phrase “under conditions that permit binding” refers to any environment that permits the desired activity, for example, conditions under which two or more molecules, such as nucleic acid molecules and/or protein molecules, can bind. Such conditions can include specific concentrations of salts and/or other chemicals that facilitate the binding of molecules.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

All publications cited herein and the subject matter for which they are cited are hereby specifically incorporated by reference in their entireties.

### EXAMPLES

The following examples are provided to illustrate certain particular features and/or embodiments of the disclosure. The examples should not be construed to limit the disclosure to the particular features or embodiments described.

### Example 1

### Introduction

Chromatin accessibility mapping is a powerful approach to identify potential regulatory elements. In the popular ATAC-seq method, Tn5 transposase inserts sequencing adapters into accessible DNA (‘tagmentation’). CUT&Tag is a tagmentation-based epigenomic profiling method in which antibody tethering of Tn5 to a chromatin epitope of interest profiles specific chromatin features in small samples and single cells. In this Example, it is demonstrated that modifying the tagmentation conditions for histone H3K4me2/3 CUT&Tag, antibody-tethered tagmentation of accessible DNA sites is redirected to produce accessible DNA maps that are indistinguishable from the best ATAC-seq maps. Thus, DNA accessibility maps can be produced in parallel with CUT&Tag maps of other epitopes with all steps from nuclei preparation to amplified sequencing-ready libraries performed in single PCR tubes in the laboratory or on a home workbench. Considering that H3K4 methylation is produced by transcription at promoters and enhancers, the method identifies transcription-coupled accessible regulatory sites. This modified CUT&Tag protocol is referred to as Cleavage Under Targeted Accessible Chromatin (CUTAC).

### Results

Streamlined CUT&Tag Produces High-Quality Datasets with Low Cell Numbers

“CUT&RUN” is a modification of Laemmli's Chromatin Immunocleavage (ChIC) method (Schmid M, et al. (2004) ChIC and ChEC; genomic mapping of chromatin proteins. Mol Cell 16:147-157), in which a fusion protein between Micrococcal Nuclease (MNase) and Protein A (pA-MNase) binds sites of antibody binding in nuclei or permeabilized cells bound to magnetic beads. Activation of MNase with Ca⁺⁺ results in targeted cleavage releasing the antibody-bound fragment into the supernatant for paired-end DNA sequencing. More recently, the Tn5 transposase was substituted for MNase in a modified CUT&RUN protocol, such that addition of Mg⁺⁺ results in a cut-and-paste “tagmentation” reaction, in which sequencing adapters are integrated around sites of antibody binding (Kaya-Okur H S, et al. (2019) CUT&Tag for efficient epigenomic profiling of small samples and single cells. Nat Commun 10:1930). In this updated method, called “CUT&Tag”, DNA purification is followed by PCR amplification, eliminating the end-polishing and ligation steps required for sequencing library preparation in CUT&RUN. Like CUT&RUN, CUT&Tag requires relatively little input material, and the low backgrounds permit low sequencing depths to sensitively map chromatin features.

A streamlined version of CUT&Tag was developed that eliminates tube transfers, so that all steps can be efficiently performed in a single PCR tube (Kaya-Okur H S, et al. (2020) Efficient low-cost chromatin profiling with CUT&Tag. Nature Protocols 15, 3264-3283). However, the suitability of the single-tube protocol for profiling low cell number samples has not been determined. During the COVID-19 pandemic, this CUT&Tag-direct protocol was adapted for implementation with minimal equipment and space requirements that uses no toxic reagents, so that it can be performed conveniently and safely even on a home workbench. To ascertain the ability of this CUT&Tag-direct protocol to produce DNA sequencing libraries at home with data quality comparable to those produced in the laboratory, frozen aliquots of native human K562 cell nuclei prepared in the laboratory were used and profiled there using the streamlined single-tube protocol. Aliquots of nuclei were thawed and serially diluted in Wash buffer from ˜60,000 down to ˜60 starting cells, where the average yield of nuclei was ˜50%. Antibodies to H3K4me3 were used, which preferentially marks nucleosomes immediately downstream of active promoters, and H3K27me3, which marks nucleosomes within broad domains of Polycomb-dependent silencing. Aliquots of nuclei were taken home and stored in a kitchen freezer, then thawed and diluted at home and profiled for H3K4me3 and H3K27me3. In both the laboratory and at home all steps were performed in groups of 16 or 32 samples over the course of a single day through the post-PCR clean-up step, treating all samples the same regardless of cell numbers. Whether produced at home or in the lab, all final barcoded sample libraries underwent the same quality control, equimolar pooling, and final SPRI bead clean-up steps in the laboratory prior to DNA sequencing.

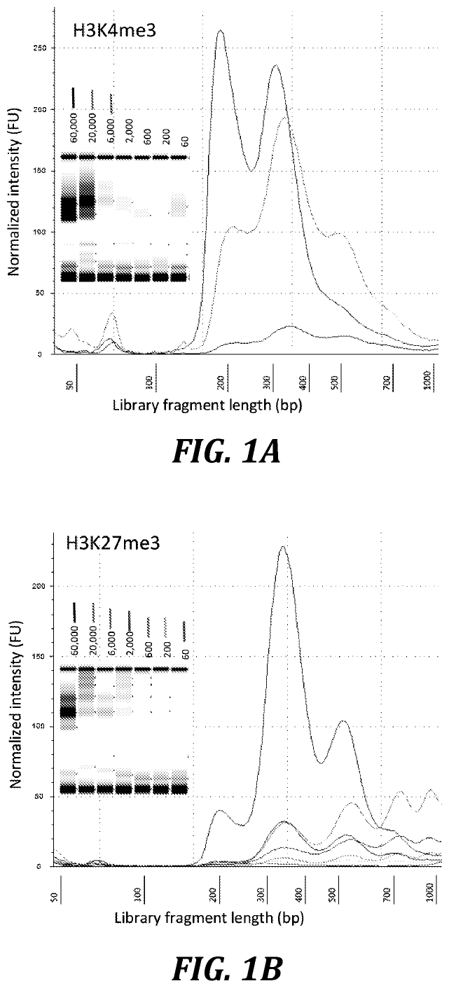

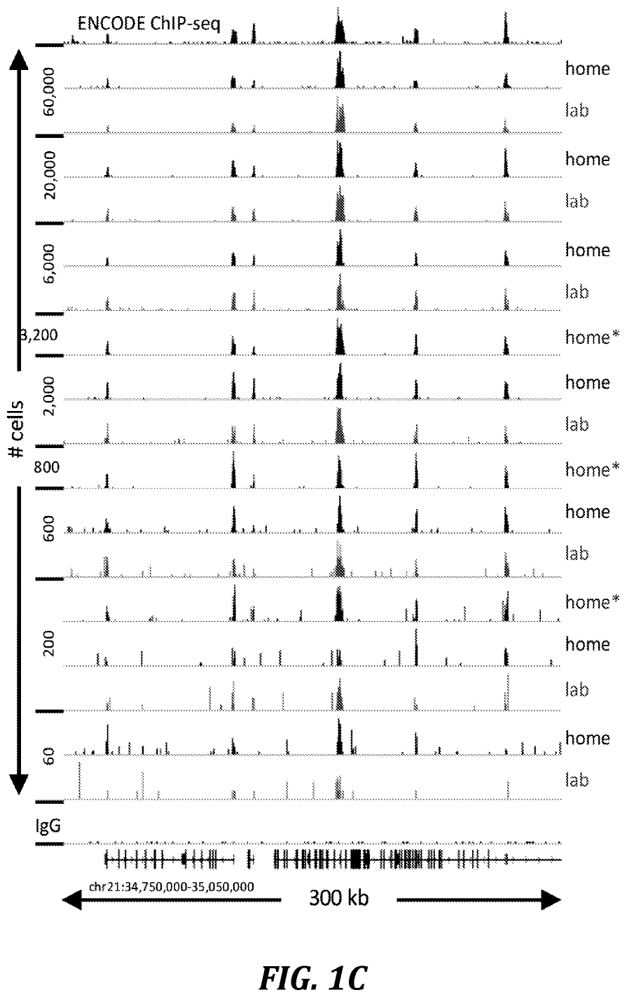

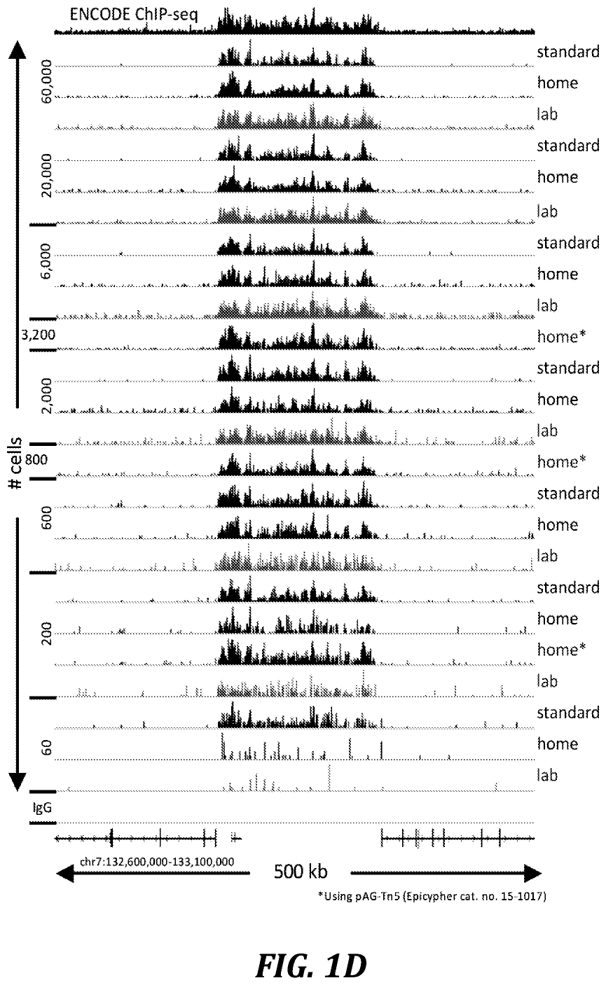

Tapestation profiles of libraries produced at home detected nucleosomal ladders down to 200 cells for H3K27me3 and nucleosomal and subnucleosomal fragments down to 2000 cells for H3K4me3 (FIG. 1A-1B). Sequenced fragments were aligned to the human genome using Bowtie2 and tracks were displayed using IGV. Similar results were obtained for both at-home and in-lab profiles for both histone modifications (FIG. 1C-1D) using pA-Tn5 produced in the laboratory, and results using commercial Protein A/Protein G-Tn5 (pAG-Tn5) were at least as good. All subsequent experiments reported here were performed at home using Epicypher pAG-Tn5, which provided results similar to those obtained using batches of homemade pA-Tn5 run in parallel.

NDRs Attract Tn5 Tethered to Nearby Nucleosomes During Low-Salt Tagmentation

Because the Tn5 domain of pA-Tn5 binds avidly to DNA, it is necessary to use elevated salt conditions to avoid tagmenting accessible DNA during CUT&Tag. High-salt buffers included 300 mM NaCl for pA-Tn5 binding, washing to remove excess protein, and tagmentation at 37° C. It has been found that other protocols based on the same principle but that do not include a high-salt wash step result in chromatin profiles that are dominated by accessible site tagmentation (Kaya-Okur H S, et al. (2020) Efficient low-cost chromatin profiling with CUT&Tag. Nature Protocols 15, 3264-3283).

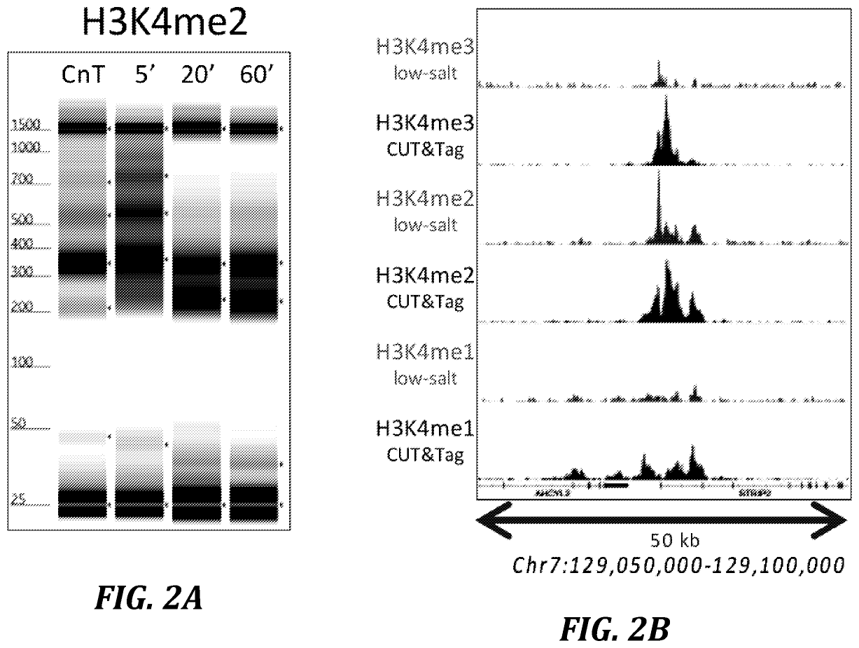

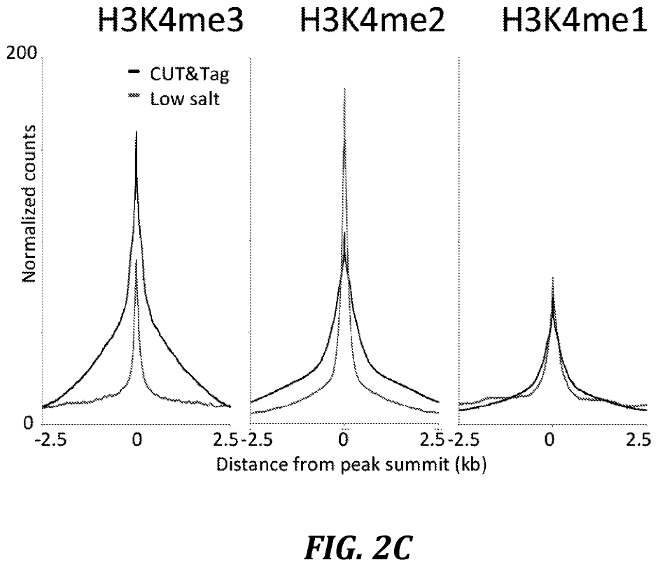

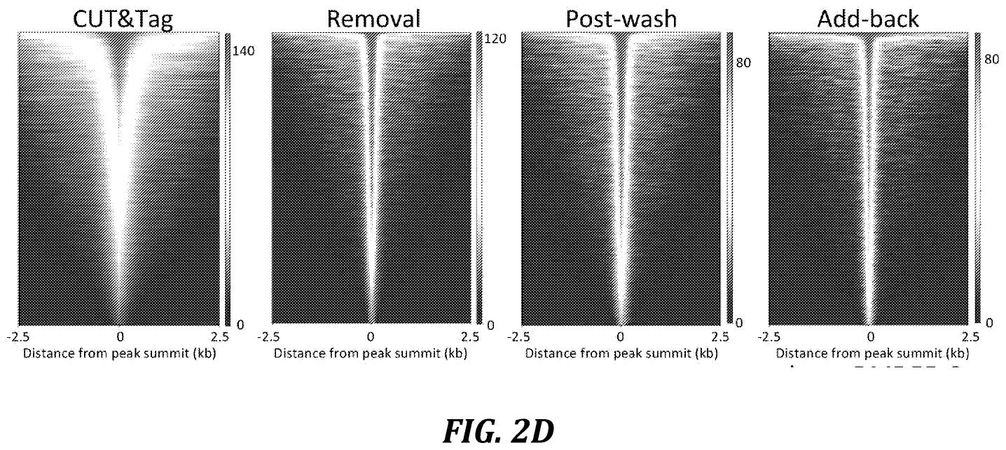

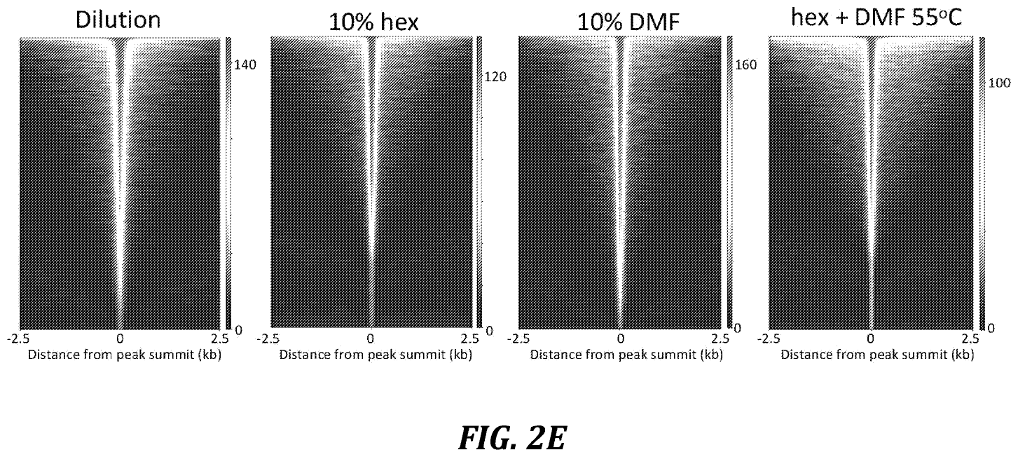

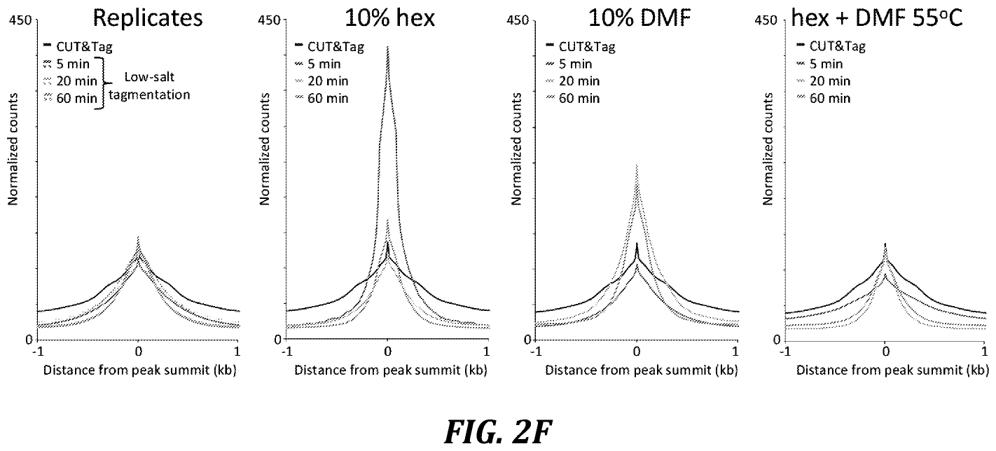

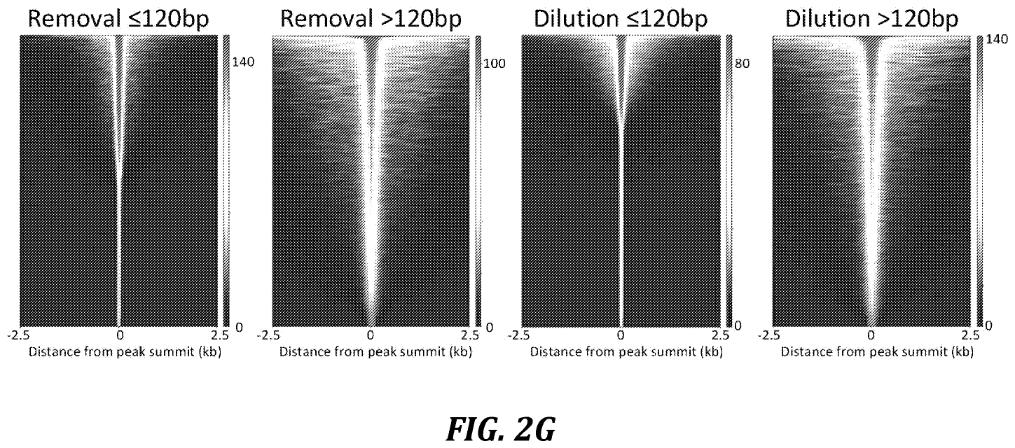

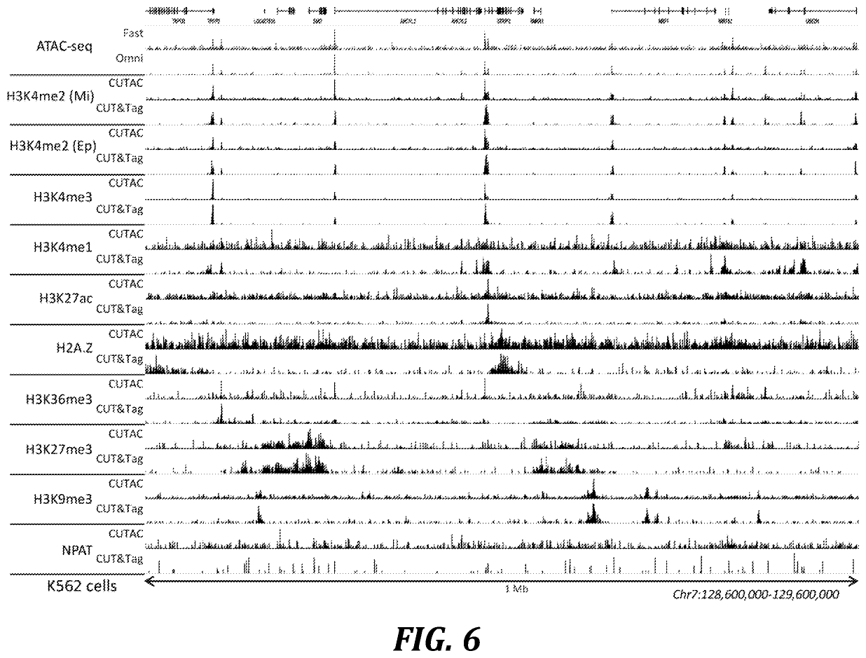

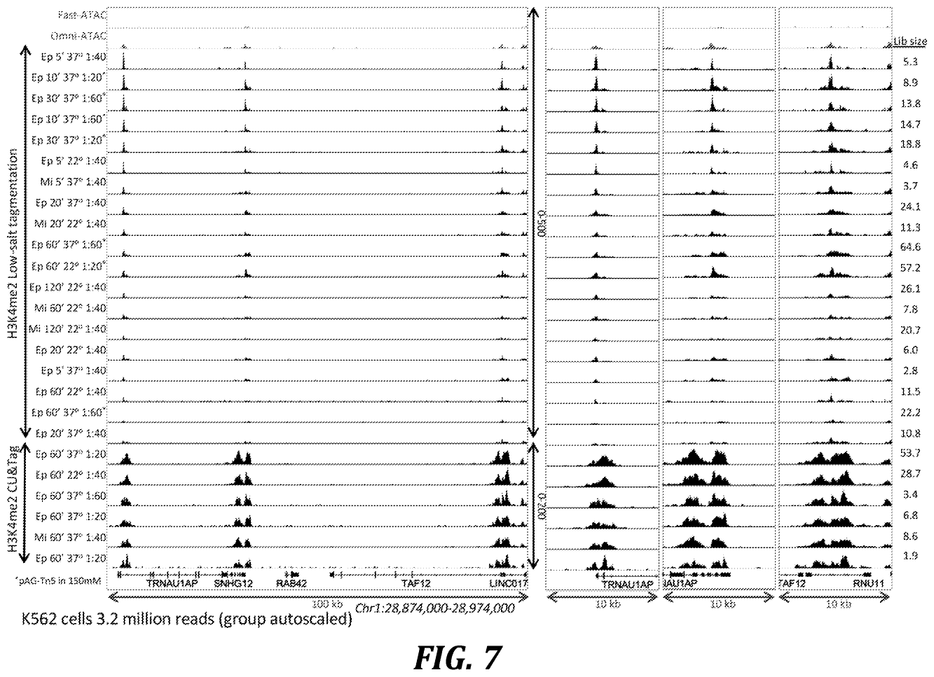

To better understand the mechanistic basis for the salt-suppression effect, pAG-Tn5 was bound under normal high-salt CUT&Tag incubation conditions, then tagmented in low salt. Either rapid 20-fold dilution with a prewarmed solution of 2 mM or 5 mM MgCl₂ or removal of the pAG-Tn5 incubation solution and addition of 50 μL 10 mM TAPS pH8.5, 5 mM MgCl₂, was used. All other steps in the protocol followed the CUT&Tag-direct protocol (Kaya-Okur H S, et al. (2020) Efficient low-cost chromatin profiling with CUT&Tag. Nature Protocols 15, 3264-3283). Tapestation capillary gel electrophoresis of the final libraries revealed that after a 20 minute incubation the effect of low-salt tagmentation on H3K4me2 CUT&Tag samples was a marked reduction in the oligo-nucleosome ladder with an increase in faster migrating fragments (FIG. 2A). CUT&Tag profiles using antibodies to most chromatin epitopes in the dilution protocol showed either little change or elevated levels of non-specific background tagmentation that obscured the targeted signal (FIG. 6), as expected considering that the high-salt wash step needed to remove unbound pAG-Tn5 had been omitted. Strikingly, under low-salt conditions, high resolution profiles of H3K4me3 and H3K4me2 showed that the broad nucleosomal distribution of CUT&Tag around promoters for these two modifications was mostly replaced by single narrow peaks (FIGS. 2B, 7).

To evaluate the generality of peak shifts MACS2 was used to call peaks, and the occupancy over aligned peak summits were plotted. For all three H3K4 methylation marks using normal CUT&Tag high-salt tagmentation conditions a bulge was observed around the summit representing the contribution from adjacent nucleosomes on one side or the other of the peak summit (FIG. 2C). In contrast, tagmentation under low salt conditions revealed much narrower profiles for H3K4me3 and H3K4me2 (˜40% peak width at half-height), less so for H3K4me1 (˜60%), which suggests that the shift is from H3K4me-marked nucleosomes to an adjacent NDR.

To determine whether free pAG-Tn5 present during the 20-fold dilution into MgCl₂, the pAG-Tn5 was removed just before adding 5 mM MgCl₂ to tagment, and again narrowing of the H3K4me2 peak was observed (FIG. 2D left two heatmaps). A narrowing was also observed if a stringent 300 mM washing step was included before low-salt tagmentation (FIG. 2D, third heatmap), which indicates that peak narrowing does not require free pAG-Tn5. However, it was observed that the peak narrowed further if following the stringent wash low-salt tagmentation included a small amount of pAG-Tn5 and incubation was extended from 20 min to 1 hr (FIG. 2D rightmost heatmap). Because Tn5 is inactive once it integrates its payload of adapters, and each fragment is generated by tagmentation at both ends, it is likely that a small amount of free pA(G)-Tn5 is sufficient to generate the additional small fragments where tethered pA(G)-Tn5 is limiting.

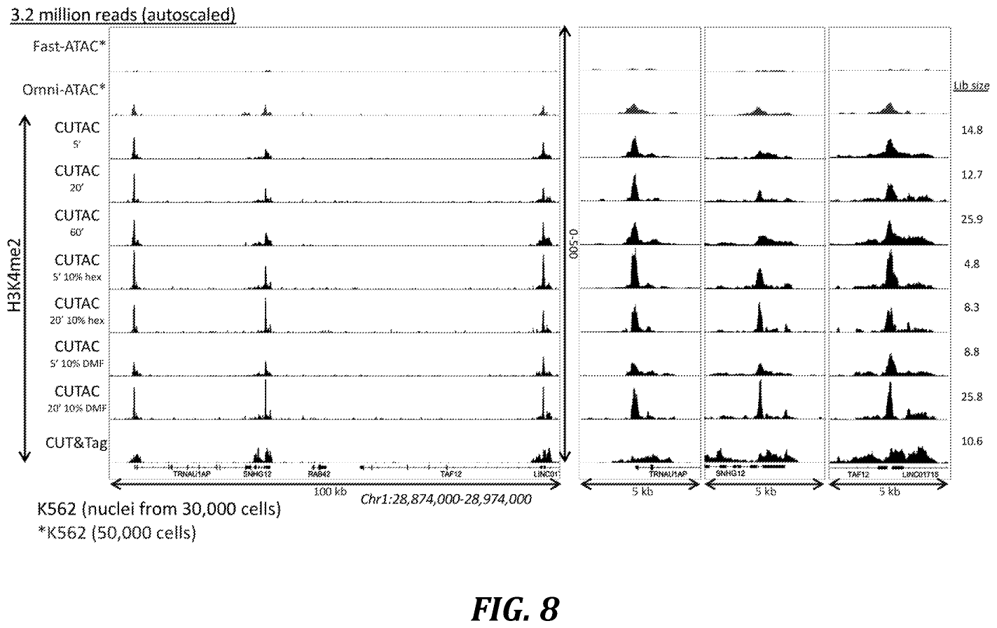

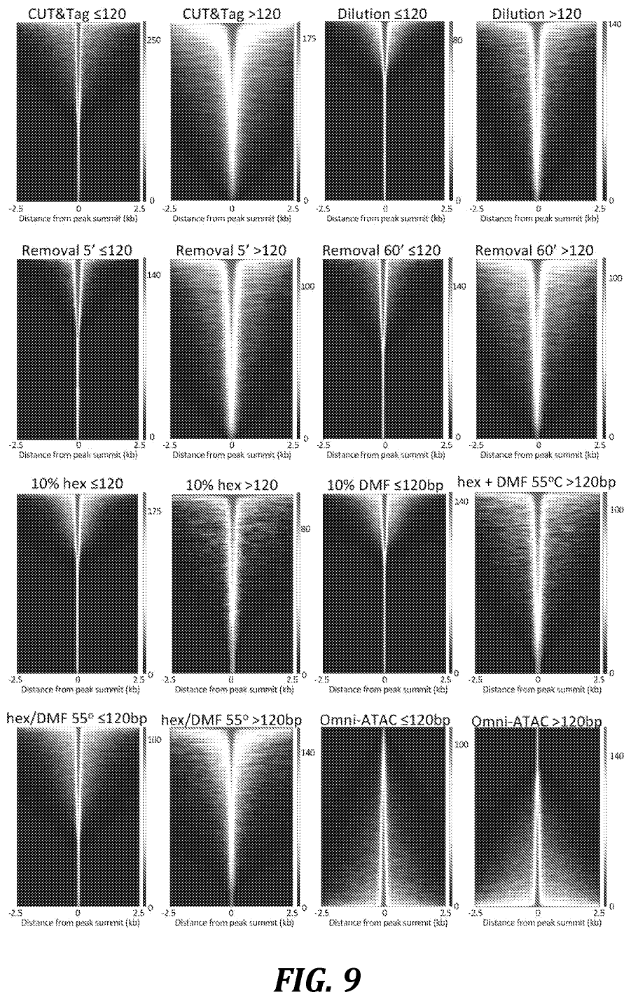

Salt ions compete with protein-DNA binding and so it was hypothesized that tagmentation in low salt resulted in increased binding of epitope-tethered Tn5 to a nearby NDR and then tagmentation. As H3K4 methylation is deposited in a gradient of tri- to di- to mono-methylation downstream of the +1 nucleosome from the transcriptional start site (TSS) (Henikoff S & Shilatifard A (2011) Histone modification: cause or cog? Trends Genet 27:389-396), it was reasoned that the closer proximity of di- and tri-methylated nucleosomes to the NDR than mono-methylated nucleosomes resulted in preferential proximity-dependent “capture” of Tn5. Consistent with this interpretation, a shift from broad to more peaky NDR profiles was observed and heatmaps by H3K4me2 low-salt tagmentation were enhanced by addition of 1,6-hexanediol, a strongly polar aliphatic alcohol, and by 10% dimethylformamide, a strongly polar amide, both of which enhance chromatin accessibility (FIGS. 2E-2F and 8). NDR-focused tagmentation persisted even in the presence of both strongly polar compounds at 55° C. Enhanced localization by chromatin-disrupting conditions suggests improved access of H3K4me2-tethered Tn5 to nearby holes in the chromatin landscape during low-salt tagmentation. Localization to NDRs is more precise for small (≤120 bp) than large (>120) tagmented fragments (FIGS. 2D, 9).

CUT&Tag Low-Salt Tagmentation Fragments Coincide with ATAC-Seq Sites

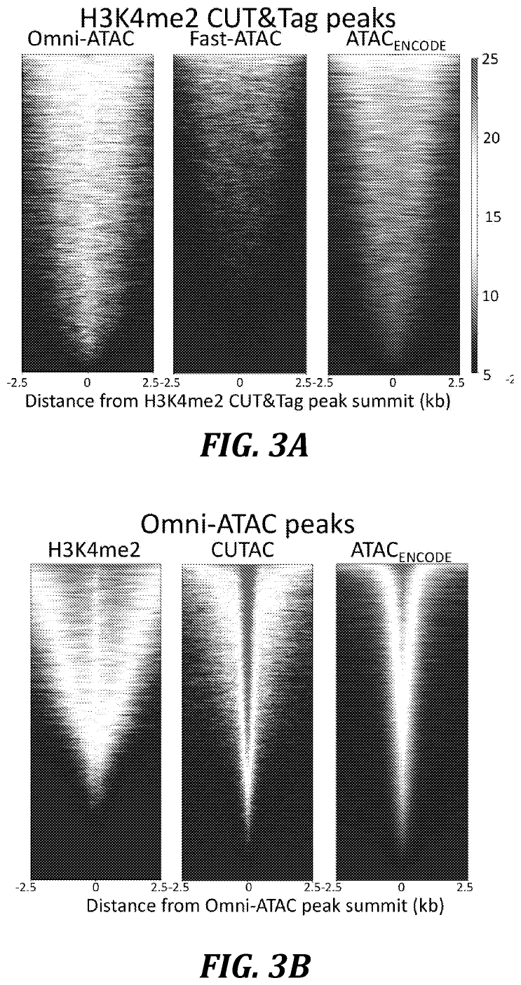

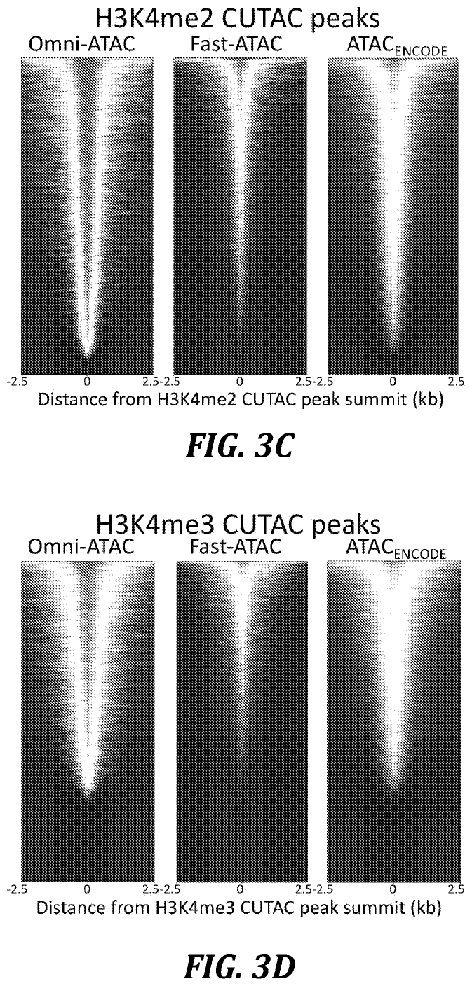

Using CUT&Tag, it was previously shown that most ATAC-seq sites are flanked by H3K4me2-marked nucleosomes in K562 cells (Kaya-Okur H S, et al. (2019) CUT&Tag for efficient epigenomic profiling of small samples and single cells. Nat Commun 10:1930). However, lining up ATAC-seq datasets over peaks called using H3K4me2 CUT&Tag data showed smeary heatmaps, reflecting the broad distribution of peak calls over nucleosome positions flanking NDRs (FIG. 3A). In contrast, alignment of ATAC-seq datasets over peaks called using low-salt tagmented CUT&Tag data produced narrow heatmap patterns for the vast majority of peaks (FIG. 3B). These observations were confirmed by the reciprocal comparisons, aligning H3K4me2 CUT&Tag datasets over peaks called from Omni-ATAC data. To reflect the close similarities between fragments released by H3K4me2-tethered low-salt tagmentation as by ATAC-seq using untethered Tn5, the low-salt H3K4me2/3 CUT&Tag tagmentation will be referred to as Cleavage Under Targeted Accessible Chromatin (CUTAC).

To further evaluate the degree of similarity between CUTAC and ATAC-seq, the ENCODE ATAC-seq dataset was aligned over peaks called using Omni-ATAC and CUTAC, where all datasets were sampled down to 3.2 million mapped fragments with mitochondrial fragments removed. Remarkably, heatmaps produced using either Omni-ATAC or CUTAC peak calls for the same ENCODE ATAC-seq data showed occupancy of ˜95% for both sets of peaks (compare right panels of FIGS. 3B and 3C). Using a window of 250 bp around the peak summit based on average peak width at half-height, ˜50% overlap was observed between ENCODE ATAC-seq peaks and peaks called from either Omni-ATAC (50.0%) or CUTAC (51.3%) data. This equivalence between H3K4me2 CUTAC and Omni-ATAC when compared to ENCODE ATAC-seq implies that CUTAC and Omni-ATAC are indistinguishable in detecting the same chromatin features. This conclusion does not hold for H3K4me3 CUTAC, because similar alignment of ENCODE ATAC-seq data resulted in only ˜75% peak occupancy (FIG. 3D), which is attributed to the greater enrichment of H3K4me3 around promoters than enhancers relative to H3K4me2.

To sensitively evaluate signal-to-noise genome-wide, peaks were called using MACS2 and calculated the Fraction of Reads in Peaks (FRiP), a data quality metric introduced by the ENCODE project (Landt S G, et al. (2012) ChIP-seq guidelines and practices of the ENCODE and modENCODE consortia. Genome Res 22:1813-1831). For both ENCODE ChIP-seq and the published CUT&RUN data, FRiP˜0.2 was measured for 3.2 million fragments, whereas for CUT&Tag, FRiP˜0.4, reflecting improved signal-to-noise relative to previous chromatin profiling methods (Kaya-Okur H S, et al. (2019) CUT&Tag for efficient epigenomic profiling of small samples and single cells. Nat Commun 10:1930). Using CUT&Tag-direct, H3K4me2 CUT&Tag FRiP=0.41 for 3.2 million fragments and ˜16,000 peaks (n=4), whereas tagmentation by dilution in 2 mM MgCl₂ resulted in FRiP=0.18 for 3.2 million fragments and ˜15,000 peaks (n=4) with similar values for tagmentation by removal [FRiP=0.21, ˜15,000 peaks (n=4)]. In add-back experiments, lower FRiP values were measured after stringent washing conditions whether or not some pAG-Tn5 was added back.

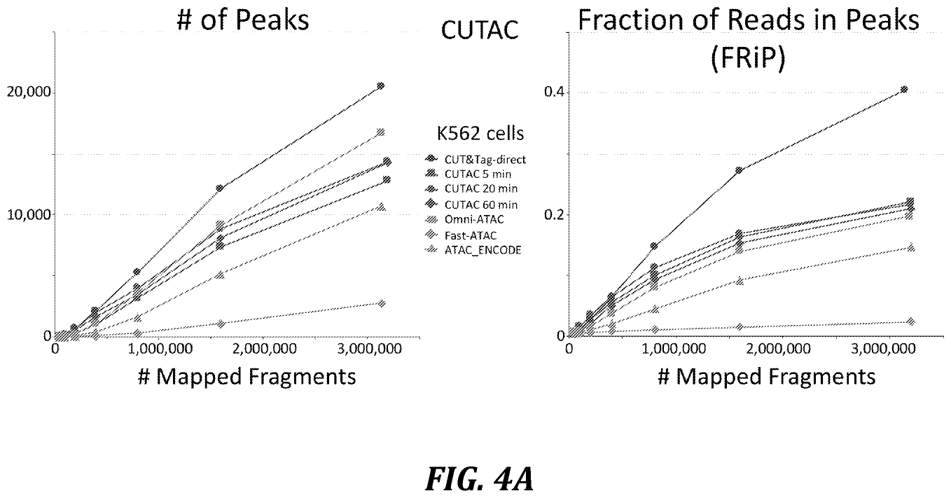

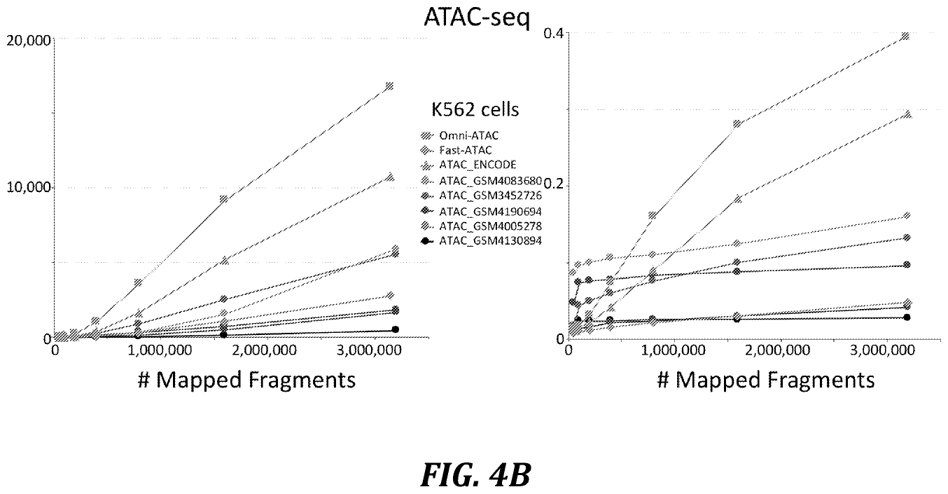

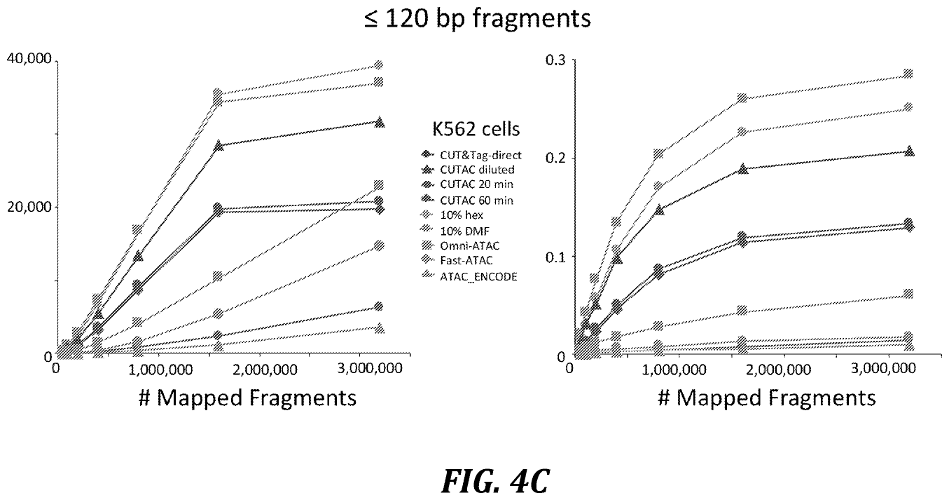

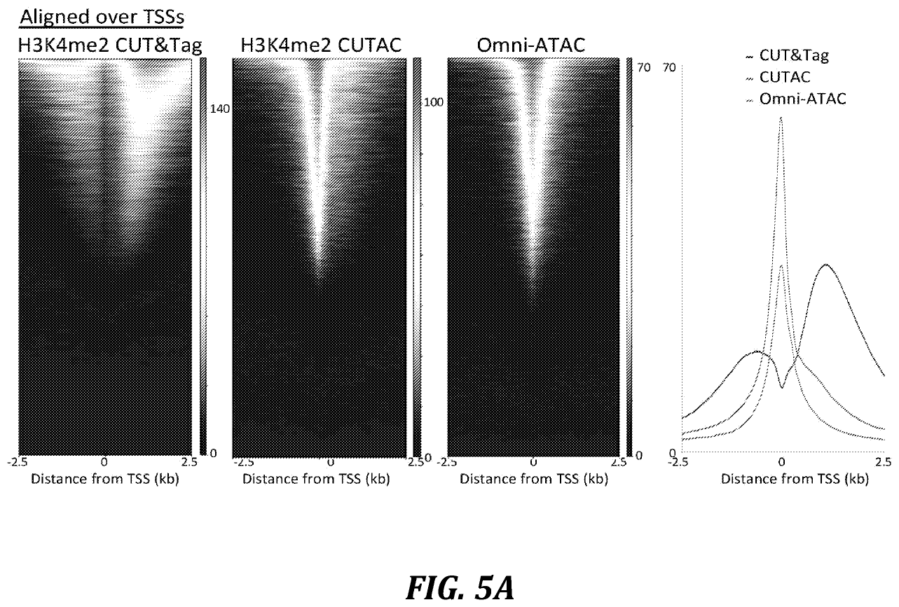

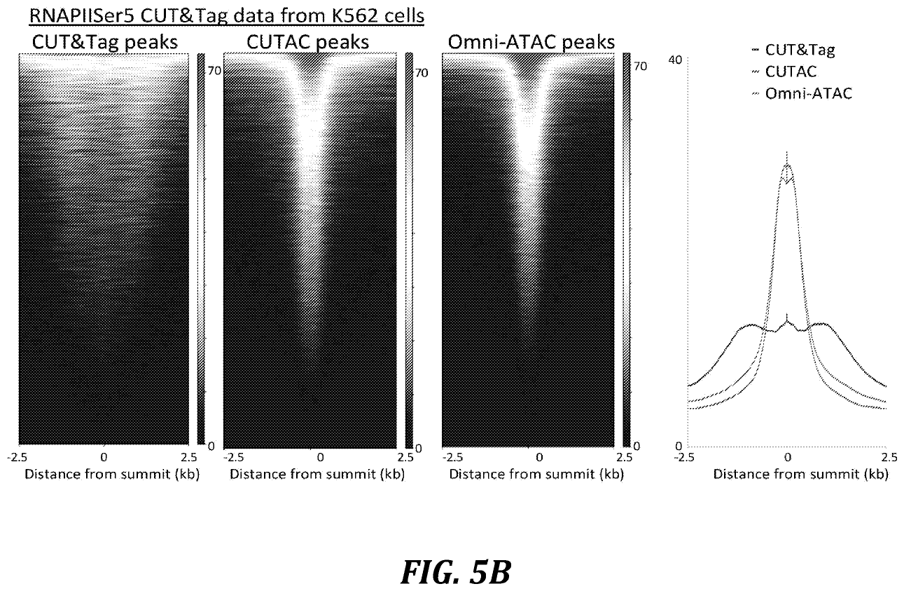

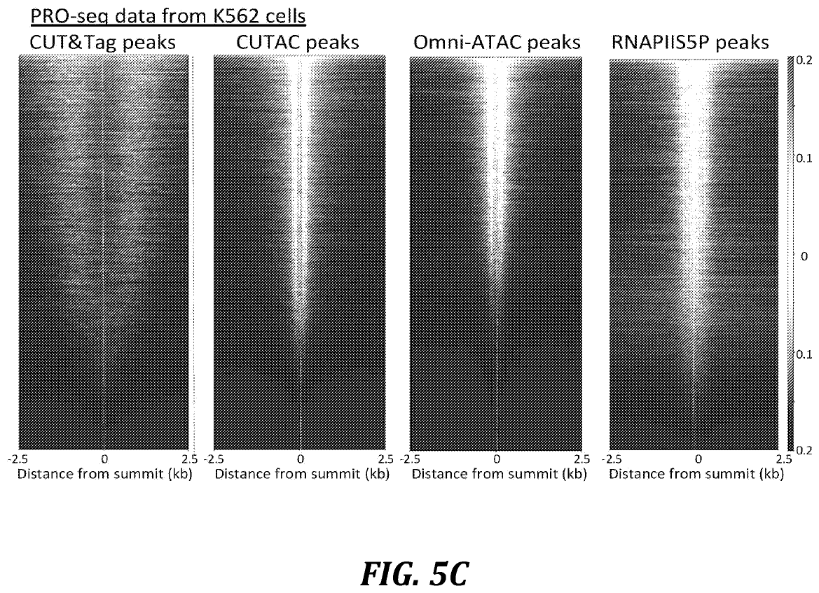

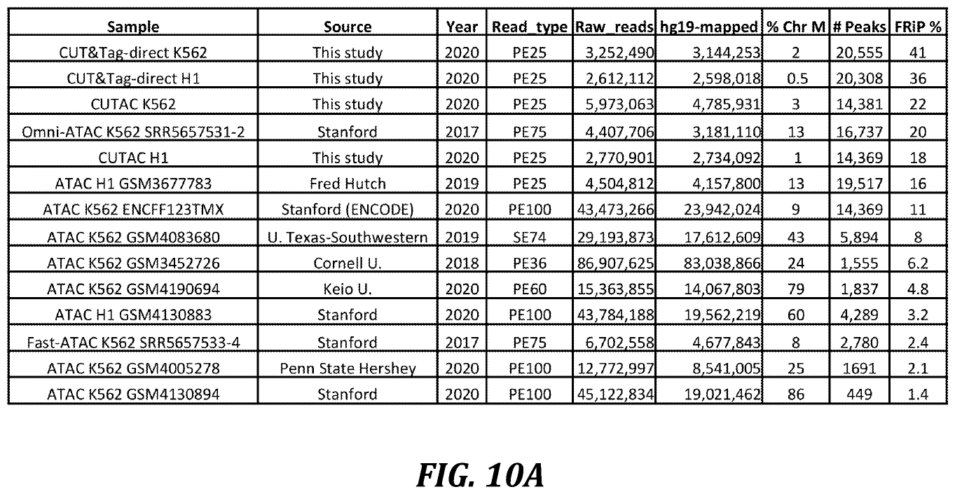

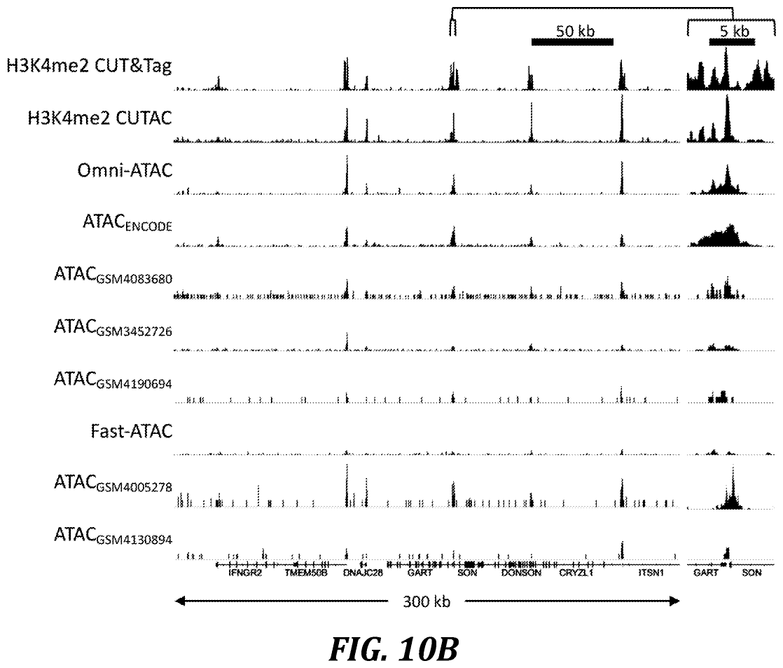

The number of peaks and FRiP values for CUTAC were also compared to those for ATAC-seq for K562 cells and it was observed that CUTAC data quality was similar to that for Omni-ATAC method (Corces M R, et al. (2017) An improved ATAC-seq protocol reduces background and enables interrogation of frozen tissues. Nat Methods 14:959-962), better than ENCODE ATAC-seq (Zhang J, et al. (2020) An integrative ENCODE resource for cancer genomics. Nat Commun 11:3696), and much better than Fast-ATAC (Corces M R, et al. (2016) Lineage-specific and single-cell chromatin accessibility charts human hematopoiesis and leukemia evolution. Nat Genet 48:1193-1203), a previous improvement over Standard ATAC-seq (Buenrostro J D, et al. (2013) Transposition of native chromatin for fast and sensitive epigenomic profiling of open chromatin, DNA-binding proteins and nucleosome position. Nat Methods 10:1213-1218) (FIG. 4A). CUTAC is relatively insensitive to tagmentation times, with similar numbers of peaks and similar FRiP values for samples tagmented for 5, 20 and 60 minutes (FIG. 4A). The robustness of CUT&Tag and CUTAC is attributed to the tethering of Tn5 to specific chromatin epitopes, so that when tagmentation goes to completion there is little untethered Tn5 that would increase background levels. When peak numbers and FRiP values were measured for ATAC-seq for K562 data deposited in the Gene Expression Omnibus (GEO) from multiple laboratories, a wide range of data quality was observed (FIG. 5B), even from very recent submissions (FIGS. 10A and 10B). This variability was attributed to the difficulty of avoiding background tagmentation by excess free Tn5 in ATAC-seq protocols and subsequent release of non-specific nucleosomal fragments (Swanson E, et al. (2020) Integrated single cell analysis of chromatin accessibility and cell surface markers. biorxiv 2020.09.04.283887).

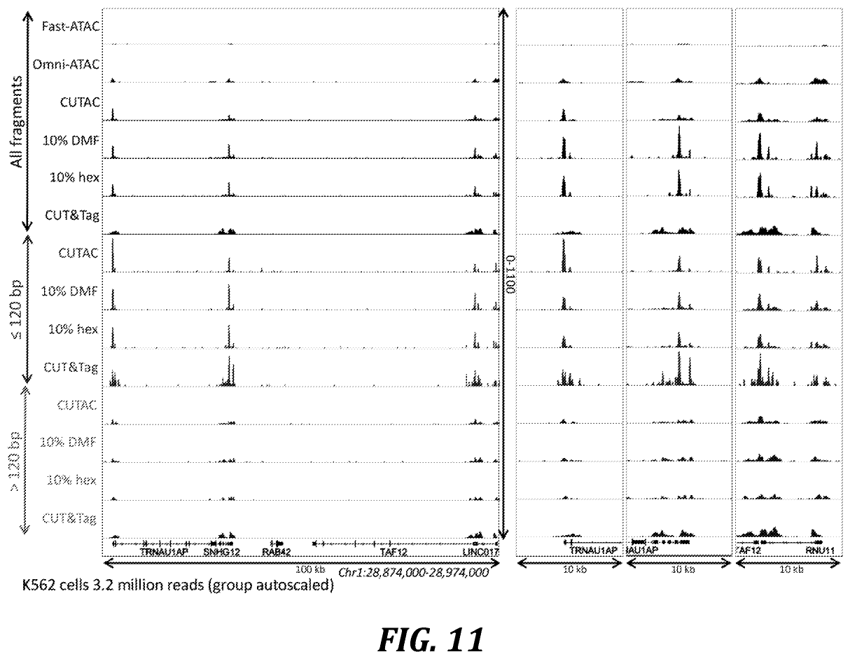

If low-salt tagmentation sharpens peaks of DNA accessibility because tethering to neighboring nucleosomes increases the probability of tagmentation in small holes in the chromatin landscape, smaller fragments would then be expected to dominate CUTAC peaks. Indeed this is exactly what is observed for heatmaps (FIGS. 10A and 10B), tracks (FIG. 11), peak calls and FRiP values (FIG. 4C). The improved resolution that results from excluding larger fragments results in more peaks with better resolution and higher FRIP values, both of which approach a maximum with fewer fragments. Also, the addition of strongly polar compounds during tagmentation provides a substantial improvement in peak calling and FRiPs (FIG. 4C, turquoise and orange curves). No improvement is seen for ATAC-seq, which indicates that tethering to H3K4me2 is critical for maximum sensitivity and resolution of DNA accessibility maps.

CUTAC Maps Transcription-Coupled Regulatory Elements

H3K4me2/3 methylation marks active transcription at promoters (Gilchrist D A, et al. (2012) Regulating the regulators: the pervasive effects of Pol II pausing on stimulus-responsive gene networks. Genes Dev 26:933-944), which raises the question as to whether sites identified by CUTAC are also sites of RNAPII enrichment genome-wide. To test this possibility, CUTAC data were first aligned at annotated promoters displayed as heatmaps or average plots and it was observed that CUTAC sites are located in the NDR between flanking H3K4me2-marked nucleosomes (FIG. 5A). CUTAC sites at promoter NDRs corresponded closely to promoter ATAC-seq sites, consistent with expectation for promoter NDRs.

To determine whether CUTAC sites are also sites of transcription initiation in general, CUT&Tag RNA Polymerase II (RNAPII) Serine-5 phosphate (RNAPIIS5P) CUT&Tag data was aligned over H3K4me2 CUT&Tag and CUTAC and Omni-ATAC peaks ordered by RNAIIS5P peak intensity. When displayed as heatmaps or average plots, CUTAC datasets display a conspicuous shift into the NDR from flanking nucleosomes (FIG. 5B).

Mammalian transcription also initiates at many enhancers, as shown by transcriptional run-on sequencing, which identifies sites of RNAPII pausing whether or not a stable RNA product is normally produced (Kaikkonen M U, et al. (2013) Remodeling of the enhancer landscape during macrophage activation is coupled to enhancer transcription. Mol Cell 51:310-325). Accordingly, RNAPII-profiling PRO-seq data for K562 cells was aligned over H3K4me2 CUT&Tag and CUTAC and Omni-ATAC sites, displayed as heatmaps and ordered by PRO-Seq signal intensity. The CUT&Tag sites showed broad enrichment centered ˜1 kb from PRO-seq signal, whereas PRO-seq signals were tightly centered around CUTAC sites, with similar results for Omni-ATAC sites (FIG. 5B). Interestingly, alignment around TSSs, RNAPIIS5P or PRO-seq data resolves immediately flanking H3K4me2-marked nucleosomes in CUT&Tag data, which is not seen for the same data aligned on signal midpoints (FIGS. 2, 4). Such alignment of +1 and −1 nucleosomes next to fixed NDR boundaries is consistent with nucleosome positioning based on steric exclusion (Kaikkonen M U, et al. (2013) Remodeling of the enhancer landscape during macrophage activation is coupled to enhancer transcription. Mol Cell 51:310-325). Moreover, the split in PRO-seq occupancies around NDRs defined by CUTAC and Omni-ATAC demonstrates that the steady-state location of most engaged RNAPII is immediately downstream of the NDR from which it initiated. About 80% of the CUTAC sites showed enrichment of PRO-Seq signal downstream, confirming that the large majority of CUTAC sites correspond to NDRs representing transcription-coupled regulatory elements.

### Discussion

The correlation between sites of high DNA accessibility and transcriptional regulatory elements, including enhancers and promoters, has driven the development of several distinct methods for genome-wide mapping of DNA accessibility for nearly two decades (Klein D C & Hainer S J (2020) Genomic methods in profiling DNA accessibility and factor localization. Chromosome Res 28:69-85). However, the processes that are responsible for creating gaps in the nucleosome landscape are not completely understood. In part this uncertainty is attributable to variations in nucleosome positioning within a population of mammalian cells such that there is only a ˜20% median difference in absolute DNA accessibility between DNaseI hypersensitive sites and non-hypersensitive sites genome-wide (Chereji R V, et al. (2019) Accessibility of promoter DNA is not the primary determinant of chromatin-mediated gene regulation. Genome Res 29:1985-1995). This suggests that DNA accessibility is not the primary determinant of gene regulation and contradicts the popular characterization of accessible DNA sites as “open” and the lack of accessibility as “closed”. Moreover, there are multiple dynamic processes that can result in nucleosome depletion, including transcription, nucleosome remodeling, transcription factor binding, and replication, so that the identification of a presumed regulatory element by chromatin accessibility mapping leaves open the question as to how accessibility was established and maintained. The disclosed CUTAC mapping method now provides a physical link between a transcription-coupled process and DNA hyperaccessibility by showing that anchoring of Tn5 to a nucleosome mark laid down by transcriptional events downstream identifies the large majority of ATAC-seq sites.

The mechanistic basis for asserting that H3K4 methylation is a transcription-coupled event is well-established (Henikoff S & Shilatifard A (2011) Histone modification: cause or cog? Trends Genet 27:389-396). In all eukaryotes, H3K4 methylation is catalyzed by SET1/MLL and related enzymes, which associate with the C-terminal domain (CTD) of the large subunit of RNAPII when Serine-5 of the tandemly repetitive heptad repeat of the CTD is phosphorylated following transcription initiation. The enrichment of dimethylated and trimethylated forms of H3K4 is presumably the result of exposure of the H3 tail to SET1/MLL during RNAPII stalling just downstream of the TSS, so that these modifications are coupled to the onset of transcription. Therefore, the present demonstration that Tn5 tethered to H3K4me2/3 histone tail residues efficiently tagments accessible sites, implies that accessibility at regulatory elements is created by events immediately downstream of transcription initiation. This conclusion is consistent with the recent demonstration that PRO-seq data can be used to accurately impute “active” histone modifications (Wang Z, et al. (2020) Accurate imputation of histone modifications using transcription. biorxiv 2020.04.08.032730). Thus CUTAC identifies active promoters and enhancers that produce enhancer RNAs, which might help explain why ˜95% of ATAC-seq peaks are detected by CUTAC and vice-versa (FIG. 4B-C).

CUTAC also provides practical advantages over other chromatin accessibility mapping methods. As it requires only a simple modification of one step in the CUT&Tag protocol, CUTAC can be performed in parallel with an H3K4me2 CUT&Tag positive control and other antibodies using multiple aliquots from each population of cells to be profiled. It is demonstrated here that three distinct protocol modifications, dilution, removal and post-wash tagmentation, provide similar high-quality results, providing flexibility that might be important for adapting CUTAC to nuclei from diverse cell types and tissues.

Although a CUT&Tag-direct experiment requires a day to perform, and ATAC-seq can be performed in a few hours, this disadvantage of CUTAC is offset by the better control of data quality with CUTAC as is evident from the large variation in ATAC-seq data quality between laboratories. In contrast, CUT&Tag is highly reproducible using native or lightly cross-linked cells or nuclei (Kaya-Okur H S, et al. (2020) Efficient low-cost chromatin profiling with CUT&Tag. Nature Protocols 15, 3264-3283), and as shown here H3K4me2 CUT&Tag maps regulatory elements with sensitivity and signal-to-noise comparable to the best ATAC-seq datasets using three protocol variations. Although H3K4me2 CUTAC datasets are somewhat noisier than H3K4me2 CUT&Tag datasets run in parallel, the combination of the two provides both highest data quality (CUT&Tag) and precise mapping (CUTAC) using the same H3K4me2 antibody. Therefore, it is expected that current CUT&Tag users and others will find the CUTAC option to be an attractive alternative to other DNA accessibility mapping methods for identifying transcription-coupled regulatory elements.

### Materials and Methods

Biological Materials

Human K562 cells were purchased from ATCC (Manassas, VA, Catalog #CCL-243) and cultured following the supplier's protocol. H1 ES cells were obtained from WiCell (Cat #WA01-lot #WB35186) and cultured following NIH 4D Nucleome guidelines (available online at data.4dnucleome.org/protocols/50f8300d-400f-4ce1-8163-42f417cbbada/). The following antibodies were used: Guinea Pig anti-Rabbit IgG (Heavy & Light Chain) antibody (Antibodies-Online ABIN101961 or Novus NBP1-72763), Rabbit anti-mouse (Abcam ab46540), H3K4me1 (Epicypher 13-0026, lot 28344001), H3K4me2 (Epicypher 13-0027 and Millipore 07-030, lot 3229364), H3K4me3 (Active Motif, 39159), H3K9me3 (Abcam ab8898, lot GR3302452-1), H3K27me3 (Cell Signaling Technology, 9733, Lot 14), H3K27ac (Millipore, MABE647), H3K36me3 (Epicypher #13-0031, lot 18344001) and NPAT (Thermo Fisher Scientific, PA5-66839). The pAG-Tn5 fusion protein used in these experiments was a gift from Epicypher, Inc. (#15-1117 lot #20142001-C1).

CUT&Tag-Direct and CUTAC

Log-phase human K562 or H1 embryonic stem cells were harvested and prepared for nuclei in a hypotonic buffer with 0.1% Triton-X100 essentially as described (Skene P J & Henikoff S (2017) An efficient targeted nuclease strategy for high-resolution mapping of DNA binding sites. eLife 6:e21856). Detailed, step-by-step nuclei preparation protocol can be found in Kaya-Okur, H. S., et al. (2019) CUT&Tag for efficient epigenomic profiling of small samples and single cells. Nat Commun 10, 1930; and Oh K S, et al. (2019) XL-DNase-seq: improved footprinting of dynamic transcription factors. Epigenetics Chromatin 12(1):30.

CUT&Tag-direct was performed as described (Kaya-Okur H S, et al. (2020) Efficient low-cost chromatin profiling with CUT&Tag. Nature Protocols 15, 3264-3283), and a detailed step-by-step protocol including the modification for CUTAC is described in Example 2, below. Except as noted, all experiments were performed on a workbench in a home laundry room. Briefly, nuclei were thawed, mixed with activated Concanavalin A beads and magnetized to remove the liquid with a pipettor and resuspended in Wash buffer (20 mM HEPES pH 7.5, 150 mM NaCl, 0.5 mM spermidine and Roche EDTA-free protease inhibitor). After successive incubations with primary antibody (1-2 hr) and secondary antibody (0.5-1 hr) in Wash buffer, the beads were washed and resuspended in pA(G)-Tn5 at 12.5 nM in 300-Wash buffer (Wash buffer containing 300 mM NaCl) for 1 hr. Incubations were performed at room temperature either in bulk or in volumes of 25-50 μL in low-retention PCR tubes. For CUT&Tag, tagmentation was performed for 1 hr in 300-Wash buffer supplemented with 10 mM MgCl₂ in a 50 μL volume. For CUTAC, tagmentation was performed in low-salt buffer with varying components, volumes and temperatures as described for each experiment in the Description of the Drawings. In “dilution” tagmentation, tubes containing 25 μL of pA(G)-Tn5 incubation solution and 2 mM or 5 mM MgCl₂ solutions were preheated to 37° C. Tagmentation solution (475 μL) was rapidly added to the tubes and incubated for times and temperatures as indicated. In “removal” tagmentation, tubes were magnetized, liquid was removed, and 50 μL of ice-cold 10 mM TAPS, 5 mM MgCl₂ was added, followed by incubation for times and temperatures as indicated. In “add-back” tagmentation, beads were washed in 500 μL 300-wash buffer as in CUT&Tag, and then 50 μL of ice-cold 10 mM TAPS, 5 mM MgCl₂ was added, supplemented with pA(G)-Tn5 and incubated at 37° C. for times as indicated.

Following tagmentation, CUT&Tag and CUTAC samples were chilled and magnetized, liquid was removed, and beads were washed in 50 μL 10 mM TAPS pH8.5, 0.2 mM EDTA then resuspended in 5 μL 0.1% SDS, 10 μL TAPS pH8.5. Following incubation at 58° C., SDS was neutralized with 15 μL of 0.67% Triton-X100, and 2 μL of 10 mM indexed P5 and P7 primer solutions were added. Tubes were chilled and 25 μL of NEBNext 2× Master mix was added with mixing. Gap-filling and 12 cycles of PCR were performed using an MJ PTC-200 Thermocycler. Clean-up was performed by addition of 65 μL SPRI bead slurry following manufacturer's instructions, eluted with 20 μL 1 mM Tris-HCl pH 8, 0.1 mM EDTA and 2 μL was used for Agilent 4200 Tapestation analysis. The barcoded libraries were mixed to achieve equimolar representation as desired aiming for a final concentration as recommended by the manufacturer for sequencing on an Illumina HiSeq 2500 2-lane Turbo flow cell.

Data Processing and Analysis

Paired-end reads were aligned to hg19 using Bowtie2 version 2.3.4.3 with options: --end-to-end--very-sensitive--no-unal--no-mixed--no-discordant--phred33-I 10-X 700. Tracks were made as bedgraph files of normalized counts, which are the fraction of total counts at each basepair scaled by the size of the hg19 genome. Peaks were called using MACS2 version 2.2.6 callpeak-f BEDPE-g hs-p le-5-keep-dup all-SPMR. Heatmaps were produced using deepTools 3.3.1. A detailed step-by-step Data Processing and Analysis Tutorial referred to as “CUT&Tag Data Processing and Analysis Tutorial” can be found online at protocols.io.

### Example 2

This Example describes an exemplary step-by-step protocol to perform the CUT&Tag-direct with a parallel performance of Cleavage Under Targeted Accessible Chromatin (CUTAC) method, which is an optimized variation of the CUT&Tag method for efficient chromatin profiling. The CUTAC variation permits hyperaccessibility mapping. This Example is presented in the context of targeting H3K4me2-labeled nucleosomes for both the CUT&Tag-direct and CUTAC methods in parallel. It will be understood that the CUTAC method can be performed individually without the CUT&Tag-direct variation. Additionally, alternative markers for transcription activity can be readily substituted for the exemplary H3K4me2 with the selection of the appropriate antibodies.

### Abstract

This method uses a modification of Bench-top CUT&Tag, which includes incubation in 0.1% SDS post-tagmentation for quantitative release of targeted fragments, followed directly by PCR with Triton-X100 to neutralize the SDS. This protocol is performed in single PCR tubes from nuclei to sequencing-ready libraries and is suitable for high throughput. The protocol has been enhanced by the addition of hyperaccessibility mapping by Cleavage Under Targeted Accessible Chromatin (CUTAC), where H3K4me2 CUT&Tag samples are tagmented in low salt for mapping of the hyperaccessible site close to the H3K4me2-labeled nucleosomes.

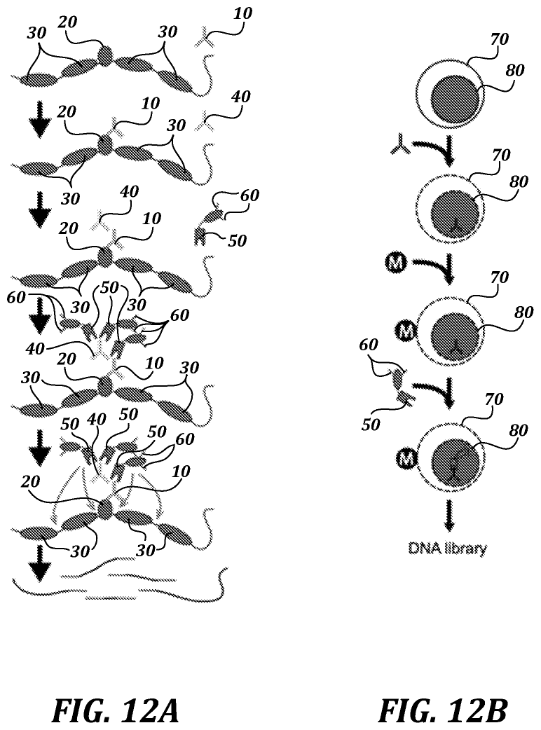

As an overview, FIGS. 12A and 12B provide a schematic overview of in situ tethering for CUT&Tag chromatin profiling, which forms the basis of CUTAC. (12A) The steps in CUT&Tag. Added antibody (10) binds to the target chromatin protein (20) between nucleosomes (30) in the genome, and the excess is washed away. A second antibody (40) is added and enhances tethering of pA-Tn5 transposome (50) at antibody-bound sites. After washing away excess transposome, addition of Mg++ activates the transposome and integrates adapters (60) at chromatin protein binding sites. After DNA purification genomic fragments with adapters at both ends are enriched by PCR. (12B) CUT&Tag is performed on a solid support. Unfixed cells (70) or nuclei (80) are permeabilized and mixed with antibody to a target chromatin protein. After addition and binding of cells to Concanavalin A-coated magnetic beads (M), all further steps are performed in the same reaction tube with magnetic capture between washes and incubations, including pA-Tn5 tethering, integration, and DNA purification.

### Reagent Setup

1. Binding buffer Mix 200 μL 1M HEPES-KOH pH 7.9, 100 μL 1M KCl, 10 μL 1M CaCl₂ and 10 μL 1M MnCl₂, and bring the final volume to 10 mL with dH₂O. Store the buffer at 4° C. for up to several months.
   Wash buffer Mix 1 mL 1 M HEPES pH 7.5, 1.5 mL 5 M NaCl, 12.5 μL 2 M spermidine, bring the final volume to 50 mL with dH₂O, and add 1 Roche Complete Protease Inhibitor EDTA-Free tablet. Store the buffer at 4° C. for up to several months.
   Antibody buffer Mix 10 μL 100×BSA with 1 mL. Wash buffer and chill on ice.
   300-wash buffer Mix 1 mL 1 M HEPES pH 7.5, 3 mL 5 M NaCl and 12.5 μL 2 M spermidine, bring the final volume to 50 mL with dH₂O and add 1 Roche Complete Protease Inhibitor EDTA-Free tablet. Store at 4° C. for up to several months.
   CUT&Tag Tagmentation solution Mix 1 mL 300-wash buffer and 10 μL 1 M MgCl₂ (to 10 mM).
   CUTAC Tagmentation solution Mix 197 μL dH₂O, 2 μL 1 M TAPS pH 8.5 and 1 μL 1 M MgCl₂ (10 mM TAPS, 5 mM MgCl₂).
   CUTAC-dilution Tagmentation solution Mix 15 mL dH₂O, 33 μL 1 M MgCl2 (2 mM MgCl₂) and preheat to 37° C.
   CUTAC-hex Tagmentation solution Mix 97 μL dH₂O, 100 μL 20% (w/v) 1,6-hexanediol, 2 μL 1 M TAPS pH 8.5 and 1 μL 1 M MgCl₂ (10 mM TAPS, 5 mM MgCl₂).
   CUTAC-DMF Tagmentation solution Mix 177 μL dH₂O, 20 μL N,N-dimethylformamide, 2 μL 1 M TAPS pH 8.5 and 1 μL 1 M MgCl₂ (10 mM TAPS, 5 mM MgCl₂).
   TAPS wash buffer Mix 1 mL dH₂O, 10 μL 1 M TAPS pH 8.5, 0.4 μL 0.5 M EDTA (10 mM TAPS, 0.2 mM EDTA)
   0.1% SDS Release solution Mix 10 μL 10% SDS and 10 μL 1 M TAPS pH 8.5 in 1 ml dH₂O
   0.67% Triton neutralization solution Mix 67 μL 10% Triton-X100+933 μL

### Prepare Concanavalin A-Coated Beads (15 Min)

2. Resuspend and withdraw enough of the ConA bead slurry such that there will be 3-5 μL for each final sample of up to ˜50,000 mammalian cells. The following is for 16 samples.
3. Transfer 85 μL ConA bead slurry into 1 mL Binding buffer in a 1.5 mL tube and mix by pipetting. Place the tube on a magnet stand to clear (30 s to 2 min).
4. Withdraw the liquid completely, and remove from the magnet stand. Add 1 mL Binding buffer and mix by pipetting.
5. Place on magnet stand to clear, withdraw liquid, and resuspend in 85 μL Binding buffer (for 5 μL per sample).

### Bind Nuclei to ConA Bead (15 Min)

6. Thaw a frozen aliquot of native nuclei at room temperature, for example by placing in a 20 ml beaker of water.
7. Transfer the thawed nuclei suspension in aliquots of no more than 50,000 starting mammalian cells to each PCR tube and mix with 3-5 μL ConA beads in thin-wall 0.5 ml PCR tubes and let sit at room temperature for 10 min. NOTE: Nuclei prepared according to the Benchtop CUT&Tag Version 3 protocol have been resuspended in Wash buffer. Beads can be added directly to the aliquot for binding and then transferred to PCR tubes such that no more than 5 μL of the original ConA bead suspension is present in each PCR tube for single-tube CUT&Tag. Using more than ˜50,000 mammalian nuclei or >5 μL Con A beads per sample may inhibit the PCR.
8. Place the tubes on a magnet stand to clear and withdraw the liquid. NOTE: In low-retention PCR tubes, surface tension will cause bead-bound cells to slide down to the bottom of the tube, so to avoid losses here and below, set the pipettor to 5 μL less than the liquid volume to be removed and use multiple draws to remove the last liquid without losing beads.

### Bind Primary Antibody (1 hr)

9. Resuspend cells in 25-50 μL Antibody buffer then 0.5 μL antibody (1:50-1:100) with gentle vortexing. For each CUTAC control sample (dilution, removal or post-wash), prepare a separate H3K4me2 tube (or split the H3K4me2 sample at the appropriate step). NOTE: For bulk processing, resuspend in Antibody buffer containing antibody (1:100) with gentle vortexing.
10. Place on a Rotator at room temperature and incubate 1-2 hr. NOTES: Volumes up to 50 μL will remain in the tube bottom by surface tension during rotation. The α-H3K4me2 antibody used for both CUT&Tag and CUTAC serves as a control evaluate success of the procedure without requiring library preparation. An optional negative control is performed by omitting the primary antibody.

### Bind Secondary Antibody (1 hr)

11. Place tubes on the magnet stand to clear. Withdraw the liquid with the pipettor set to 5 μL less than the volume to be removed.
12. Mix the secondary antibody 1:100 in Wash buffer and squirt in 50 μL per sample while gently vortexing to allow the solution to dislodge the beads from the sides.
13. Place the tubes on a Rotator and rotate at room temperature for 30 min.
14. After a quick spin (<500×g), place the tubes on a magnet stand to clear and withdraw the liquid with the pipettor set to 5 μL less than the volume to be removed. NOTE: Surface tension causes beads slide down the side of low-retention PCR tubes, and removing the last drop can result in loss of beads. To avoid this, remove the 50 μL volume with 3 successive draws using a 20 μL tip with the pipettor set for maximum volume.
15. After a quick spin, replace on the magnet stand and withdraw the last drop with a 20 μL pipette tip.
16. With the tubes still on the magnet stand, carefully add 500 μL Wash buffer. The surface tension will cause the beads to slide up along the side of the tube closest to the magnet.
17. Slowly withdraw 470 μL with a 1 mL pipette tip without disturbing the beads, followed by complete liquid removal using multiple draws with a 20 μL pipettor. NOTE: To withdraw the liquid, set the pipettor to 470 μL, and keep the plunger depressed while lowering the tip to the bottom. The liquid level will rise to near the top completing the wash. Then ease off on the plunger until the liquid is withdrawn, and remove the pipettor. This will leave behind a small drop of liquid that is removed with a 20 μL pipettor, avoiding significant bead loss.
18. Replace on magnet and withdraw the liquid with a 20 μL pipettor using multiple draws. Proceed immediately to the next step.
19. Remove tubes from the magnet and squirt in 50 μL Wash buffer, vortex gently followed by a quick spin.
   Bind pA-Tn5 Adapter Complex (1.5 hr)
20. Mix pAG-Tn5 pre-loaded adapter complex in 300-wash buffer following the manufacturer's instructions. NOTE: For CUT&Tag using Epicypher pAG-Tn5 (cat. no. 15-1117) dilute 1:20 as recommended, except for CUTAC, which is optimal at 1:40.
21. Squirt in 25-50 μL per sample of the pA-Tn5 mix while vortexing and invert by rotation to allow the solution to dislodge most or all of the beads. NOTE: When using the recommended Macsimag magnet stand, dislodging the beads can be done by removing the plexiglass tube holder from the magnet, and with fingers on top to prevent the tubes from opening up or falling out, invert by rotating sharply a few times.
22. After a quick spin (<500×g), place the tubes on a Rotator at room temperature for 1 hr.
23. For a CUTAC by dilution H3K4me2 sample, hold at RT until Step 27. Continue with other tubes.
24. After a quick spin place the tubes on a magnet stand to clear and withdraw the liquid with a 20 μL pipettor using multiple draws. For a CUTAC by removal sample, hold on ice until Step 29.
25. With the tubes still on the magnet stand, add 500 μL 300-wash buffer.
26. Slowly withdraw the liquid with a 1 mL pipette tip as in Step 17.
27. After a quick spin, place the tubes on a magnet stand to clear and withdraw the liquid with a 20 μL pipettor using multiple draws. Proceed immediately to Step 32. For a CUTAC post-wash sample proceed immediately to Step 31.
   CUTAC by Dilution (Performed in Parallel with Other Samples)
28. Prewarm CUTAC by dilution tube at 37° C. in a 0.5 ml PCR tube heating block and squirt in 500 μL prewarmed 2 mM MgCl₂. Incubate 20 min. NOTE: The degree of tagmentation will vary depending on the number of nuclei and other factors, but can be controlled by varying tagmentation times. Longer tagmentation will increase yield, but reduce signal-to-noise.
29. Chill the tube and skip to Step 34.
   CUTAC by Removal (Performed in Parallel with Other Samples)
30. Add 50 μL 10 mM TAPS 5 mM MgCl₂ with gentle vortexing and incubate at 37° C. for 20 min. NOTE: This step is critical to remove most but not all of the excess pA-Tn5 and avoid high background levels of cleavage. Washing will over-deplete the remaining unbound enzyme, and reduce recovery of small CUTAC fragments.
31. Chill the tube and skip to Step 34.
   CUTAC Post-Wash (Performed in Parallel with Other Samples)
32. Resuspend the bead/nuclei pellet in 25-50 μL CUTAC tagmentation solution (5 mM MgCl₂, 10 mM TAPS) while vortexing or inverting by rotation to allow the solution to dislodge most or all of the beads as in Step 20. Proceed to Step 33.

### Tagmentation and Particle Release (2.5 hr)

33. Resuspend the bead/nuclei pellet in 25-50 μL tagmentation solution while vortexing or inverting by rotation to allow the solution to dislodge most or all of the beads as in Step 20.
34. After a quick spin (<500×g), incubate at 37° C. for 1 hr in a PCR cycler with heated lid. Hold at 8° C.
35. Place tubes on a magnet stand and withdraw the liquid with a 20 μL pipettor using multiple draws then resuspend the beads in 50 μL TAPS wash and invert by rotation as in Step 20.
36. After a quick spin, replace tubes on the magnet stand and withdraw the liquid with a 20 μL pipettor using multiple draws.
37. Resuspend the beads in 5 μL 0.10% SDS Release solution using a fresh 20 μL pipette tip to dispense while wetting the sides of the tubes to recover the fraction of beads sticking to the sides. NOTE: Twirling the tube back and forth rapidly between thumb and finger will effectively wet the sides of the tube, followed by a quick spin to bring most of the beads to the bottom.
38. Incubate at 58° C. for 1 hr in a PCR cycler with heated lid to release pA-Tn5 from the tagmented DNA.

### PCR (1 hr)

39. To the PCR tube containing the bead slurry add 15 μL Triton neutralization solution+2 μL of 10 μM Universal or barcoded i5 primer+2 μL of 10 μM uniquely barcoded i7 primers, using a different barcode for each sample. Vortex on full and place tubes in metal tube holder on ice.
40. Add 25 μL NEBnext (non-hot-start), vortex to mix, followed by a quick spin.
41. Mix, quick spin and place in Thermocycler and begin cycling program with heated lid:
   Cycle 1: 58° C. for 5 min (gap filling)
   Cycle 2: 72° C. for 5 min (gap filling)
   Cycle 3: 98° C. for 30 sec
   Cycle 4: 98° C. for 10 sec
   Cycle 5: 60° C. for 10 sec
   Repeat Cycles 4-5 11 times
   72° C. for 1 min and hold at 8° C.
   NOTE: To minimize the contribution of large DNA fragments and excess primers, PCR can be performed for no more than 12 cycles, preferably with a 10 s 60-63° C. combined annealing/extension step. The cycle times are based on using a conventional Peltier cycler (e.g., BioRad/MJ PTC200), in which the ramping times (3° C./sec) are sufficient for annealing to occur as the sample cools from 98° C. to 60° C. Therefore, the use of a rapid cycler with a higher ramping rate will require either reducing the ramping time or other adjustments to assure annealing. Do not add extra PCR cycles to see a signal by capillary gel electrophoresis (e.g. Tapestation). If there is no nucleosomal ladder for the H3K27me3 positive control, it may be assumed that CUT&Tag failed, but observing no signal for a sparse chromatin protein such as a transcription factor is normal, and the barcoded sample can be concentrated for mixing with the pool of barcoded samples for sequencing. Extra PCR cycles reduce the complexity of the library and may result in an unacceptable level of PCR duplicates.

### Post-PCR Clean-Up (30 Min)

42. After tubes have cooled, remove from the cycler and add 1.3 volume (65 μL) SPRI bead slurry, mixing by pipetting up and down.
43. Quick spin and let sit at room temperature 5-10 min.
44. Place on magnet 5 min to allow the beads to clear before withdrawing the liquid. While still on the magnet stand add 200 μL 80% ethanol.
45. Withdraw the liquid with a pipette to the bottom of the tube, and add 200 μL 80% ethanol.
46. Withdraw the liquid and after a quick spin, remove the remaining liquid with a 20 μL pipette. Do not air-dry the beads, but proceed immediately to the next step.
47. Remove from the magnet stand, add 22 μL 10 mM Tris-HCl pH 8 and vortex on full. Let sit at least 5 min.
48. Place on the magnet stand and allow to clear.
49. Remove the liquid to a fresh 1.5 ml tube with a pipette.

### Tapestation Analysis and DNA Sequencing (Outsource)

50. Determine the size distribution and concentration of libraries by capillary electrophoresis using an Agilent 4200 TapeStation with D1000 reagents or equivalent.
51. Mix barcoded libraries to achieve equal representation as desired aiming for a final concentration as recommended by the manufacturer. After mixing, perform an SPRI bead cleanup if needed to remove any residual PCR primers.
52. Perform paired-end Illumina sequencing on the barcoded libraries following the manufacturer's instructions. For maximum economy, paired-end PE25 is more than sufficient for mapping to large genomes.

### Data Processing and Analysis

53. Paired-end reads are aligned to hg19 using Bowtie2 version 2.3.4.3 with options: --end-to-end--very-sensitive--no-unal--no-mixed--no-discordant--phred33-I 10-X 700. For mapping E. coli carry-over fragments, the --no-overlap--no-dovetail options can also be used to avoid possible cross-mapping of the experimental genome to that of the carry-over E. co/i DNA that is used for calibration. Tracks are made as bedgraph files of normalized counts, which are the fraction of total counts at each basepair scaled by the size of the hg19 genome. To calibrate samples in a series for samples done in parallel using the same antibody counts of E. co/i fragments carried over can be used with the pA-Tn5 the same as one would for an ordinary spike-in. A sample script in Github (Cut-and-Run/Spike_in_Calibration.csh) can be used to calibrate based on either a spike-in or E. co/i carry-over DNA.
54. The CUT&Tag Data Processing and Analysis Tutorial available on Protocols.io provides step-by-step guidance for mapping and analysis of CUT&Tag sequencing data. Most data analysis tools used for ChIP-seq data, such as bedtools, Picard and deepTools, can be used on CUT&Tag data. Analysis tools designed specifically for CUT&RUN/Tag data include the SEACR peak caller also available as a public web server, CUT&RUNTools and henipipe.

### Example 3

This example describes another illustrative embodiment of the disclosed CUTAC method. In this embodiment, the CUTAC is directed to the initiation form of RNA Polymerase II, and so provides a more direct measure of transcriptional regulation. This approach results in a better signal-to-noise than using anti-sH3K4 methylation antibodies and is better than the best ATAC-seq data.

### Abstract

Cleavage Under Targets & Tagmentation (CUT&Tag) was previously introduced as an epigenomic profiling method in which antibody tethering of the Tn5 transposase to a chromatin epitope of interest maps specific chromatin features in small samples and single cells. With CUT&Tag, intact cells or nuclei are permeabilized, followed by successive addition of a primary antibody, a secondary antibody, and a chimeric Protein A-Transposase fusion protein that binds to the antibody. Addition of Mg⁺⁺ activates the transposase and inserts sequencing adapters into adjacent DNA in situ. Subsequently, CUT&Tag was adapted in a method called CUTAC (described in Examples 1 and 2) to also map chromatin accessibility by optimizing the transposase activation conditions when using histone H3K4me2, H3K4me3 or Serine-5-phosphorylated RNA Polymerase II antibodies. Using these antibodies, the tagmentation of accessible DNA sites was redirected to produce chromatin accessibility maps with exceptionally high signal-to-noise and resolution. All steps from nuclei to amplified sequencing-ready libraries are performed in single PCR tubes using non-toxic reagents and inexpensive equipment, making the simplified strategy for simultaneous chromatin profiling and accessibility mapping suitable for the lab, home/mobile/satellite workbench, or classroom. A schematic illustration of the CUTAC and CUT&Tag protocols is provided in FIG. 13.

### Background

Mapping of DNA accessibility in the chromatin landscape was first described 45 years ago with the observation of DNaseI hypersensitivity at transcriptionally active loci. Because DNaseI preferentially cleaves genomic regions that are depleted of nucleosomes, and regulatory elements are bound by non-histone chromatin proteins rather than nucleosomes, DNaseI hypersensitive site mapping has since been used to characterize the genetic regulatory landscape. Other enzymatic probes of chromatin accessibility include Micrococcal Nuclease (MNase), restriction endonucleases, transposases, and DNA methyltransferases. Hypersensitive site mapping became more routine with the introduction of genome-wide read-out platforms, beginning with microarrays and later short-read DNA sequencing. Chromatin accessibility was also mapped using physical fragmentation and differential recovery of cross-linked chromatin, the basis for FAIRE and Sono-Seq. In recent years, the most popular chromatin accessibility mapping method has been ATAC-seq, in which the Transposon 5 (Tn5) cut-and-paste transposition reaction inserts sequencing adapters in the most accessible genomic regions (tagmentation). Because tagmentation creates sequencing libraries simultaneous with insertion into accessible sites, ATAC-seq is simple and fast, and successively improved ATAC-seq protocols have enhanced its popularity.

Despite the utility of chromatin accessibility mapping, the mechanistic basis for chromatin accessibility itself has remained incompletely understood. In contrast to the simplistic designation of chromatin as being “open” or “closed”, recent work has shown that the median difference between an accessible and a non-accessible site in DNA is estimated to be only ˜20%, with no sites completely accessible or inaccessible in a population of cells. To better understand this nuanced interpretation of chromatin accessibility, the Cleavage Under Targets & Tamentation (CUT&Tag) method was applied for antibody-tethered in situ tagmentation of chromatin to explore the mechanistic basis for chromatin accessibility (Example 1). CUT&Tag uses a fusion protein between Protein A, which binds to the chromatin-bound antibody, and Tn5, which binds to adjacent DNA, and tagmentation occurs upon activation with Mg⁺⁺. To suppress artifactual tagmentation of untargeted accessible chromatin, all steps from pA-Tn5 fusion protein binding through tagmentation were performed in the presence of 300 mM NaCl, which reduces non-specific DNA binding of the transposase. See Example 1. In the course of optimizing a simplified single-tube protocol, CUT&Tag-direct, it was serendipitously observed that reducing the ionic concentration during antibody-targeted tagmentation greatly increased the tendency of tethered Tn5 to tagment accessible chromatin near particular histone modifications (Example 1). Preferential tagmentation of accessible chromatin only occurred when using antibodies against H3K4me2 and H3K4me3 and not for other histone modifications or variants. Because H3K4me2 flanks both promoters and enhancers genome-wide, the attraction of antibody-tethered Tn5 to nearby accessible DNA regions shifted the preferred sites of tagmentation from the nucleosomes bordering the Nucleosome-Depleted Region (NDR) to the NDR itself. Remarkably, practically all transcription-coupled accessible sites corresponded to ATAC-seq sites and vice-versa, upstream of paused RNA Polymerase II (RNAPII). Because of the close correspondence between the resulting “CUTAC” (Cleavage Under Targeted Accessible Chromatin) maps and DNaseI and ATAC-seq chromatin accessibility maps, it concluded that chromatin accessibility is driven by RNAPII transcriptional initiation (Example 1), supporting suggestions that active promoters and enhancers are characterized by the same regulatory architecture.

In the initial CUTAC study, described in Example 1, three different modifications of the CUT&Tag-direct protocol were described for accessible site mapping: tagmentation in MgCl₂ with a 20-fold dilution of 300 mM NaCl and pA-Tn5 (or commercial pAG-Tn5 with both Protein A and Protein G IgG specificities), removal of excess pAG-Tn5 before low-salt tagmentation, and low-salt tagmentation following the 300 mM wash step. Post-wash tagmentation was adopted, which follows the same steps as in the original CUT&Tag-direct protocol (Kaya-Okur, H. S., et al. (2020). Efficient low-cost chromatin profiling with CUT&Tag. Nat Protoc 15(10): 3264-3283), changing only the tagmentation buffer composition. As reported here, the application of this CUTAC protocol to the initiation form of RNAPII results in precise chromatin accessibility maps with exceptionally high signal-to-noise. The improvement obtained by tethering to the transcriptional machinery itself further supports the transcription-coupled basis for chromatin accessibility at enhancers and promoters.

CUT&Tag and CUTAC can be performed simultaneously in a single day from previously frozen native or lightly cross-linked nuclei through to purified sequencing-ready libraries, with all steps carried out in single PCR tubes. A simplified protocol is presented where all steps from nuclei to purified sequencing-ready libraries are amenable for performance on a home benchtop using surplus equipment and non-toxic reagents. See the schematic overview illustrated in FIG. 13. The CUTAC results using an antibody to the Serine-5-phosphorylated initiation form of the repeated heptameric C-terminal domain of the largest RNAPII subunit (RNAPIIS5P) compare favorably with the best ATAC-seq data while providing a genome-wide map of the initiation form of RNAPII. The simplicity and affordability of the protocol make it equally suitable for a laboratory, home or classroom environment.

### Materials and Reagents

1. Disposable tips (e.g., Rainin 1 ml, 200 μl, 20 μl)
2. Disposable centrifuge tubes for reagents (15 ml or 50 ml)
3. Standard 1.5 ml microfuge tubes
4. 0.5 ml maximum recovery PCR tubes (e.g., Fisher, catalog number: 14-222-294)
5. Phosphate-buffered saline (Fisher cat. no. BP3994)
6. 16% (w/v) formaldehyde (10×1 ml ampules, Thermo-Fisher, catalog number: 28906)
7. 1.25 M glycine (Sigma-Aldrich, catalog number: G7126)
8. Dimethyl sulfoxide (DMSO; Sigma-Aldrich, catalog number: D4540)
9. Cell culture (e.g., human K562 cells)
10. Concanavalin A (ConA)-coated magnetic beads (Bangs Laboratories, catalog number: BP531)
11. Distilled, deionized, or RNAse-free H₂O (dH₂O; e.g., Promega, catalog number: P1197)
12. 1 M Hydroxyethyl piperazineethanesulfonic acid pH 7.9 (HEPES (K⁺); Sigma-Aldrich, catalog number: H3375)
13. 1 M Manganese Chloride (MnCl₂; Sigma-Aldrich, catalog number: 203734)
14. 1 M Calcium Chloride (CaCl₂); Fisher, catalog number: BP510)
15. 1 M Potassium Chloride (KCl; Sigma-Aldrich, catalog number: P3911)
16. Roche Complete Protease Inhibitor EDTA-Free tablets (Sigma-Aldrich, catalog number: 5056489001)
17. 1 M Hydroxyethyl piperazineethanesulfonic acid pH 7.5 (HEPES (Na⁺); Sigma-Aldrich, catalog number: H3375)
18. 5 M Sodium chloride (NaCl; Sigma-Aldrich, catalog number: 55150-1L)
19. 2 M Spermidine (Sigma-Aldrich, catalog number: S0266)
20. 0.5 M Ethylenediaminetetraacetic acid (EDTA; Research Organics, catalog number: 3002E)
21. 200× Bovine Serum Albumen (BSA; NEB, catalog number: B9001S)
22. Antibody to an epitope of interest for CUT&Tag. Because in situ binding conditions are more like those for immunofluorescence (IF) than those for ChIP, it is suggested to choose IF-tested antibodies if CUT&RUN/Tag-tested antibodies are not available.
23. CUTAC control antibody to RNA Polymerase II Phospho-Rpb1 CTD Serine-5 phosphate (RNAPIIS5P) or histone H3K4me2. Excellent results have been obtained with these rabbit monoclonal antibodies: Phospho-Rpb1 CTD (Ser5) (Cell Signalling Technology, catalog number: 13523 (D9N51)) H3K4me2 (catalog number: 13-0027)
24. Secondary antibody, e.g., guinea pig α-rabbit antibody (Antibodies-Online, catalog number: ABIN101961) or rabbit α-mouse antibody (Abcam, catalog number: ab46540)
25. Protein A/G-Tn5 (pAG-Tn5) fusion protein loaded with double-stranded adapters with 19mer Tn5 mosaic ends (Epicypher, catalog number: 15-1117)
26. 1 M Magnesium Chloride (MgCl₂; Sigma-Aldrich, catalog number: M8266-100G)
27. 1 M [tris(hydroxymethyl)methylamino] propanesulfonic acid (TAPS) pH 8.5 (with NaOH)
28. 1,6-hexanediol (Sigma-Aldrich, catalog number: 240117-50G)
29. N,N-dimethylformamide (Sigma-Aldrich, catalog number: D-8654-250 ml)
30. NEBNext 2×PCR Master mix (ME541L)
31. PCR primers: 10 μM stock solutions of i5 and i7 primers with unique barcodes [see, e.g., Buenrostro, J. D. et al., Nature 523:486 (2015)] in 10 mM Tris pH 8. Standard salt-free primers may be used. Nextera or NEBNext primers are not recommended.]
32. 10% Sodium dodecyl sulfate (SDS; Sigma-Aldrich, catalog number: L4509)
33. 10% Triton X-100 (Sigma-Aldrich, catalog number: X100)
34. SPRI paramagnetic beads (e.g., HighPrep PCR Cleanup Magbio Genomics, catalog number: AC-60500)
35. 10 mM Tris-HCl pH 8.0
36. Ethanol (Decon Labs, catalog number: 2716)
37. Nuclei Extraction 1 (NE1) buffer (see Recipes)
38. Wash buffer (see Recipes)
39. Binding buffer (see Recipes)
40. Antibody buffer (see Recipes)
41. 300-wash buffer (see Recipes)
42. CUTAC Tagmentation buffer (see Recipes)
43. TAPS wash buffer (see Recipes)
44. 0.1% SDS Release solution (see Recipes)
45. 0.67% Triton neutralization solution (see Recipes)

### Equipment

1. −80° C. freezer
2. Chilling device (e.g., metal heat blocks on ice or cold packs in an ice cooler)
3. Pipettors (e.g., Rainin Classic Pipette 1 ml, 200 μl, 20 μl, and 10 μl)
4. Strong magnet stand (e.g., Miltenyi Macsimag separator, catalog number: 130-092-168)
5. Vortex mixer (e.g., VWR Vortex Genie)
6. Mini-centrifuge (e.g., VWR Model V)
7. Tube rotator (e.g., Barnstead/Thermolyne 400110)
8. PCR thermocycler (e.g., Bio-Rad/MJ PTC-200)

The methodology and requirements for equipment are amenable for mobile and satellite (including home) workstations, including typical laboratory settings. A typical experiment begins by mixing cells with activated ConA beads in up to 32 single PCR tubes, with all liquid changes performed on the magnet stand. The only tube transfer is the removal of the purified sequencing-ready libraries from the SPRI beads to fresh tubes for Tapestation analysis and DNA sequencing. The total time from thawing frozen nuclei until elution from SPRI beads is ˜8 h.

### Software

1. Bowtie2 (Langmead B, Salzberg S. Fast gapped-read alignment with Bowtie 2. Nature Methods. 2012, 9:357-359.)
2. Calibration script available at github.com/Henikoff/Cut-and-Run/blob/master/spike_in_calibration.csh

### Procedure

A. Prepare and (optionally) lightly fix nuclei and cryopreserve (1 h in the lab)
1. Harvest fresh culture(s) in a conical centrifuge tube (15 ml or 50 ml) at room temperature and count cells.
2. Centrifuge for 3 min at 600×g in a swinging bucket rotor at room temperature and drain the liquid.
3. Resuspend in 1 volume of PBS (relative to starting culture) at room temperature by pipetting up and down.
4. Centrifuge for 3 min at 600×g in a swinging bucket rotor at room temperature and drain the liquid.
5. Resuspend in 1/2 volume (relative to starting culture) of ice-cold NE1 buffer with gentle vortexing. Let sit on ice for 10 min.
6. Centrifuge for 4 min at 1,300×g at 4° C. in a swinging bucket rotor and drain liquid by pouring off and inverting onto a paper towel for a few seconds.
7. Resuspend in 1/2 volume of PBS. For unfixed nuclei, skip to Step A11.
8. While gently vortexing, add 16% formaldehyde to 0.1% (e.g., 62 μl to 10 ml) and incubate at room temperature for 2 min. Note: Light fixation reduces the tendency of cells or nuclei to clump in the 300-wash buffer, but can interfere with the binding of some antibodies, reducing yield.
9. Stop cross-linking by adding 1.25 M glycine to twice the molar concentration of formaldehyde (e.g., 600 μl to 10 ml).
10. Centrifuge for 4 min at 1,300×g at 4° C. and drain the liquid by pouring off and inverting onto a paper towel for a few seconds.
11. Resuspend in Wash buffer to a concentration of ˜1 million cells per ml. Check nuclei using a ViCell or cell counter slide.
12. Nuclei may be slowly frozen by aliquoting 900 μl into cryogenic vials containing 100 μl of DMSO, mixed well, then placed in a Mr. Frosty container filled to the line with isopropanol and placed in a −80° C. freezer overnight and stored at −80° C. long term. Note: It has been found that good results are obtained using native or cross-linked cells even after being stored in the freezer compartment of a side-by-side refrigerator for >6 months.
   B. Prepare Concanavalin A-coated beads (15 min)
1. Resuspend and withdraw enough of the ConA bead slurry, ensuring that there will be 3.5 μl for each final sample of up to ˜50,000 mammalian cells, which yield ≥50% K562 nuclei using this protocol. Transfer the ConA bead slurry into 1 ml of Binding buffer in a 1.5 ml tube. Note: This protocol has been used for up to 16 samples (60 μl beads) in 1 ml or 32 samples (120 μl beads) in 2 ml Binding buffer (in a 2 ml tube).
2. Mix by pipetting. Place the tube on a magnet stand to clear (˜1 min).
3. Withdraw the supernatant completely and remove the tube from the magnet stand. Add 1 ml Binding buffer and mix by pipetting up and down.
4. Place on the magnet stand to clear, remove and discard the supernatant, and resuspend in 60 μl Binding buffer (3.5 p1 per sample).
   C. Bind nuclei to ConA beads (15 min)
1. Thaw a frozen aliquot of nuclei at room temperature, for example by placing in a 20 ml beaker of water. Note: The CUTAC control can use either native or lightly cross-linked nuclei, preferably prepared as previously described (Kaya-Okur et al., 2020). Do not use whole cells, which require a detergent and may also inhibit the PCR.
2. Transfer the thawed nuclei suspension in aliquots of no more than ˜50,000 starting mammalian cells to each thin-wall 0.5 ml PCR tube and mix with 3.5 μl ConA beads. Attach to the Tube rotator and rotate at room temperature for 10 min. Note: Nuclei prepared according to the recommended protocol (Kaya-Okur et al., 2020) have been resuspended in Wash buffer. Beads can be added directly to the aliquot for binding and then transferred to PCR tubes, ensuring that no more than 5 μl of the original ConA bead suspension is present in each PCR tube for single-tube CUT&Tag. Using more than ˜50,000 mammalian nuclei or >5 μl Con A beads per sample may inhibit the PCR.
3. Place the tubes on the magnet stand to clear and remove and discard the supernatant. Note: In low-retention PCR tubes, surface tension will cause bead-bound cells to slide down to the bottom of the tube at this step. To avoid beads being aspirated with the supernatant, set the pipette to a volume that is 5 μl less than the total volume to be removed. Use a careful second draw with a 20 μl pipette tip and remove as much supernatant as possible, without aspirating beads.
   D. Bind primary antibody (1 h)
1. For each CUT&Tag and CUTAC sample, mix the primary antibody 1:50-1:100 with Antibody buffer. Resuspend beads in 25 μl per sample with gentle vortexing. Note: 1:50-1:100 antibody dilutions were used by default or the manufacturer's recommended concentration for immunofluorescence. CUTAC works best using either an RNA Polymerase II CTD-phosphorylated antibody (Ser5P>Ser2P/Ser5P>Ser2P) or an α-H3K4me2 antibody. α-H3K4me3 also works but is less efficient and is depleted at enhancer sites. Several antibodies to other histone epitopes have been tested, including α-H3K4me1, α-H3K36me3, α-H3K27ac, and α-H2A.Z but all have failed.
2. Place on a rotator at room temperature and incubate 1-2 h.
   Notes:
   a. Volumes up to 50 μl will remain in the tube bottom by surface tension during rotation, avoiding the need for a quick spin before the next step.
   b. After incubation, the tubes can be stored overnight at 4° C.
   c. An optional negative control is performed by omitting the primary antibody.
      E. Bind secondary antibody (1 h)
1. Place tubes on the magnet stand to clear and remove and discard the supernatant. Note: Protein in the antibody solution improves bead adherence to the tube wall, allowing for complete removal of the liquid without dislodging the beads by doing two successive draws with a 20 μl pipettor set for maximum volume while being careful not to dislodge the beads by surface tension during the second draw.
2. Mix the secondary antibody 1:100 in Wash buffer and add 25 μl per sample while gently vortexing to allow the solution to dislodge the beads from the sides. Notes:
   a. Calculate how much volume of dilutedAntibody is necessary by multiplying the number of samples by 30 μl (which is 25 μl per sample plus overage for pipetting).
   b. The secondary antibody step is required for CUT&Tag to increase the number of Protein A binding sites for each bound antibody. It was observed that without the secondary antibody the efficiency is very low.
3. Place the tubes on a rotator and rotate at room temperature for 0.5-1 h.
4. After a quick spin (<500×g or just enough to remove the liquid from the sides of the tube), place the tubes on the magnet stand to clear and remove and discard the supernatant with two successive draws, using a 20 μl tip with the pipettor set for maximum volume.
5. With the tubes still on the magnet stand, carefully add 500 μl of Wash buffer. The surface tension will cause the beads to slide up along the side of the tube closest to the magnet.
6. Slowly remove 470 μl of supernatant with a 1 ml pipette tip without disturbing the beads. Note: To remove the supernatant, set the pipettor to 470 p1l, and keep the plunger depressed while lowering the tip to the bottom. The liquid level will rise to near the top completing the wash. Then ease off on the plunger until the liquid is withdrawn, and remove the pipettor. During liquid removal, the surface tension will drag the beads down the tube. A small drop of liquid that is left behind will be removed in the next step.
7. After a quick spin (<500×g or just enough to remove the liquid from the sides of the tube), place the tubes back into the magnet stand and remove the remaining supernatant with a 20 μl pipettor over multiple times if necessary, to remove the entire supernatant without disturbing the beads. Proceed immediately to the next step.
   F. Bind pA-Tn5 adapter complex (1.5 h)
1. Mix pAG-Tn5 pre-loaded adapter complex in 300-wash buffer following the manufacturer's instructions.
2. Pipette in 25 μl per sample of the pA-Tn5 mix while vortexing and invert by rotation to ensure that beads adhering to the sides near the top of the top are resuspended. Note: When using the recommended Macsimag magnet stand, dislodging the beads after resuspending in pA-Tn5 can be done by removing the plexiglass tube holder from the magnet and, with fingers on top to prevent the tubes from opening or falling out, inverting by rotating sharply a few times.
3. After a quick spin (<500×g), place the tubes on a rotator at room temperature for 1-2 h.
4. After incubating in the rotator, perform a quick spin and place the tubes in the magnet stand.
5. Carefully remove the supernatant using a 20 μl pipettor twice to avoid disturbing the beads.
6. With the tubes still on the magnet stand, add 500 μl of the 300-wash buffer.
7. Slowly withdraw 470 μl with a 1 ml pipette tip without disturbing the beads as in Step D6.
8. After a quick spin, place the tubes back on the magnet stand and remove and discard the supernatant with a 20 μl pipettor using multiple draws. Proceed immediately to the next step.
   G. Tagmentation and particle release (2.5 h) (FIG. 13)
1. Tagmentation:
   a. CUT&Tag samples only: Resuspend the bead/nuclei pellet in 50 μl CUT&Tag Tagmentation buffer (10 mM MgCl₂ in 300-wash buffer) while vortexing or inverting by rotation to allow the solution to dislodge most or all the beads as in Step E2.
   b. CUTAC samples only: Resuspend the bead/nuclei pellet in 50 μl of either CUTAC-tag or CUTAC-hex Tagmentation buffer while vortexing or inverting by rotation to allow the solution to dislodge most or all the beads as in Step D6. Note: 10% 1,6-hexanediol or N,N-dimethylformamide compete for hydrophobic interactions and result in improved tethered Tn5 accessibility and library yield at the expense of slightly increased background.
2. After a quick spin (<500×g), incubate at 37° C. for 1 h (20 min for CUTAC) in a PCR cycler with a heated lid. Hold at 8° C.
3. Place tubes on the magnet stand and remove and discard the supernatant with a 20 μl pipettor using multiple draws, then resuspend the beads in 50 μl TAPS wash buffer and invert by rotation as in Step D6.
4. After a quick spin, place tubes on the magnet stand and remove and discard the supernatant with a 20 μl pipettor using multiple draws.
5. Resuspend the beads in 5 μl 0.1% SDS Release solution using a fresh 20 μl pipette tip to dispense while wetting the sides of the tubes to recover the fraction of beads sticking to the sides. Note: Rolling the tube back and forth rapidly between thumb and forefinger while brushing the pipette tip along the sides of the tube will effectively wet the beads, followed by a quick spin to bring most of the beads to the bottom.
6. After a quick spin (<500×g), incubate at 58° C. for 1 h in a PCR cycler with heated lid to release pA-Tn5 from the tagmented DNA.

### H. PCR (1 h)

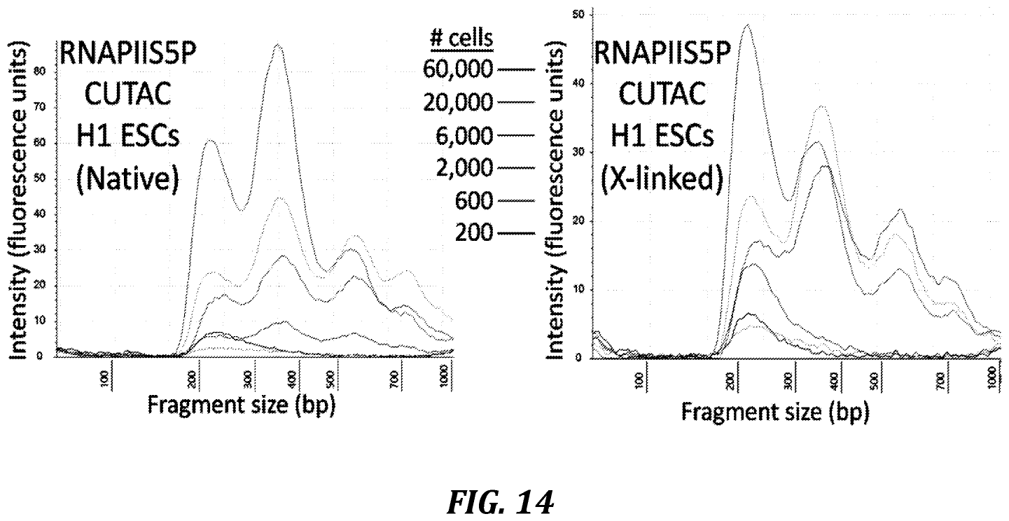

1. To the PCR tube containing the bead slurry, add 15 μl of Triton neutralization solution+2 μl of 10 μM Universal or barcoded i5 primer+2 μl of 10 μM uniquely barcoded i7 primers, using a different barcode for each sample. Vortex on full speed and place tubes in the metal tube holder on ice. Note: Exemplary indexed primers are described by Buenrostro, J D., et al. (2013). Transposition of native chromatin for fast and sensitive epigenomic profiling of open chromatin, DNA-binding proteins and nucleosome position. Nat Methods 10(12): 1213-1218. Nextera or NEB primers are not recommended as they might not anneal efficiently using this PCR protocol.
2. Add 25 μl NEBnext (non-hot start), vortex to mix, and perform a quick spin. Place the tubes immediately in the thermocycler and proceed immediately with the PCR.
3. Begin the cycling program with a heated lid on the thermocycler:
   a. Cycle 1: 58° C. for 5 min (gap filling)
   b. Cycle 2: 72° C. for 5 min (gap filling)
   c. Cycle 3: 98° C. for 30 s
   d. Cycle 4: 98° C. for 10 s
   e. Cycle 5: 60° C. for 10 s
   f. Repeat Cycles 4-5 11 times
   g. 72° C. for 1 min and hold at 8° C.
   Notes:
   a. To minimize the contribution of large DNA fragments and excess primers, the PCR should be performed for no more than 12-14 cycles, preferably with a 10 s 60-63° C. combined annealing/extension step as described above in Step H3e.
   b. The cycle times are based on using a conventional Peltier cycler (e.g., Bio-Rad/MJ PTC200), in which the ramping times (3° C./s) are sufficient for annealing to occur as the sample cools from 98° C. to 60° C. Therefore, the use of a rapid cycler with a higher ramping rate will require either reducing the ramping time or other adjustments to assure annealing.
   c. Do not add extra PCR cycles to see a signal by capillary gel electrophoresis (e.g., Tapestation). If there is no nucleosomal ladder for the H3K27me3 positive control, one may assume that CUT&Tag failed, but observing no signal for a sparse chromatin protein such as a transcription factor is normal, and the barcoded sample can be concentrated for mixing with the pool of barcoded samples for sequencing. Extra PCR cycles reduce the complexity of the library and may result in an unacceptably high level of PCR duplicates.
   d. Cycle 3 (98° C.) may be extended from 30 sec to 5 min for cross-linked samples to ensure complete cross-link reversal.
      I. Post-PCR clean-up (30 min)
1. After the PCR program ends, remove tubes from the thermocycler and add 65 μl of SPRI beads (ratio of 1.3 μl of SPRI beads to 1 μl of PCR product). Mix by pipetting up and down.
2. Let sit at room temperature 5-10 min.
3. Place on the magnetic stand for a few minutes to allow the solution to clear.
4. Remove and discard the supernatant.
5. Keeping the tubes in the magnetic stand, add 200 μl of 80% ethanol.
6. Completely remove and discard the supernatant.
7. Repeat Steps 15-16.
8. Perform a quick spin and remove the remaining supernatant with a 20 μl pipette, avoiding air drying the beads by proceeding immediately to the next step.
9. Remove from the magnet stand, add 22 μl 10 mM Tris-HCl pH 8 and vortex at full speed. Let sit for 5 min to 1 h.
10. Place on the magnet stand and allow to clear.
11. Remove the liquid to a new 1.5 ml tube with a pipette, avoiding transfer of beads.
   J. Tapestation analysis (FIG. 14) and DNA sequencing
1. Determine the size distribution and concentration of libraries by capillary electrophoresis using an Agilent 4200 TapeStation with D1000 reagents or equivalent. Note: Tapestation was used to quantify and estimate library concentration to dilute each library to 2 nM before pooling based on fragment molarity in the 175-1,000 bp range. The concentration 2 nM has been determined empirically as the optimal library concentration used in the HiSeq by the Fred Hutch Genomics Shared Resource.
2. Mix barcoded libraries to achieve equal representation as desired aiming for a final concentration as recommended by the manufacturer. After mixing, perform an SPRI bead cleanup if needed to remove any residual PCR primers.
3. Perform paired-end Illumina sequencing on the barcoded libraries following the manufacturer's instructions. For maximum economy, paired-end PE25 is more than sufficient for mapping to large genomes. Note: Using paired-end 25×25 sequencing on a HiSeq 2-lane rapid run flow cell, ˜300 million total mapped reads, or ˜3 million per sample when there are 96 samples mixed to obtain approximately equal molarity, were obtained.

### Data Analysis

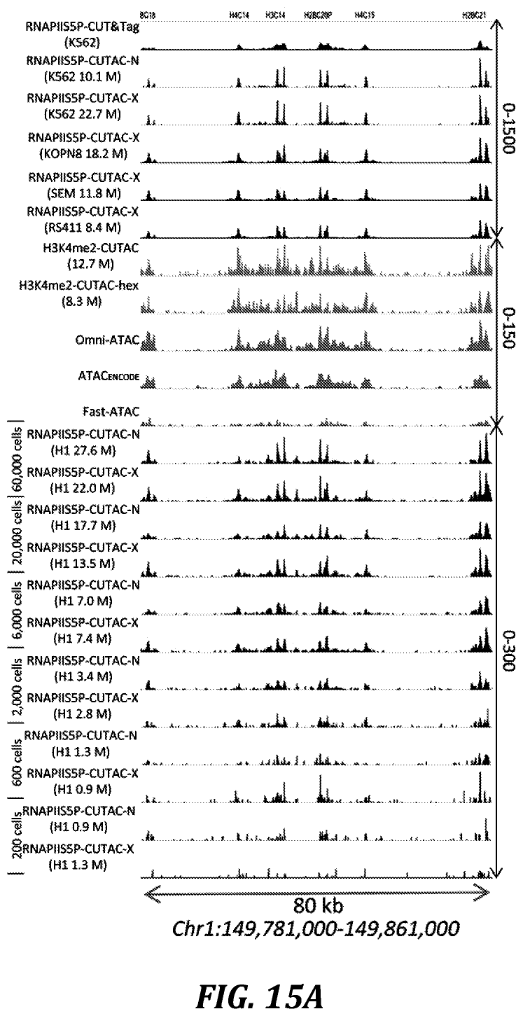

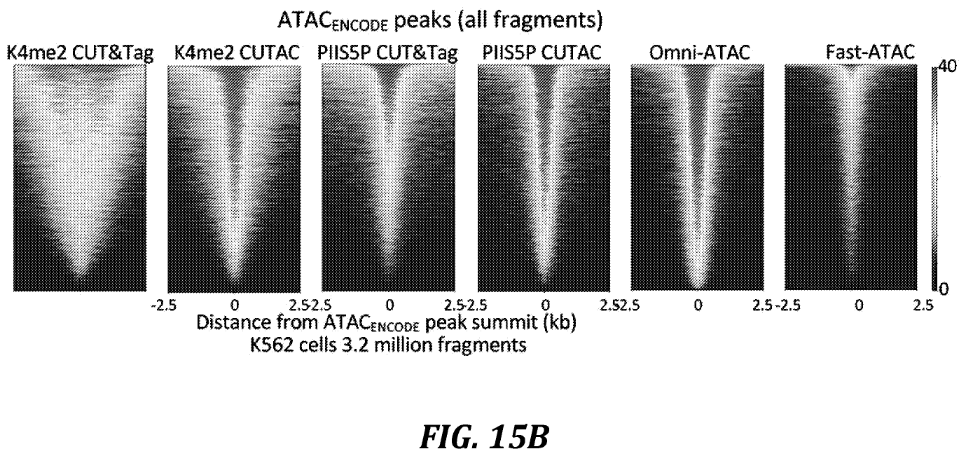

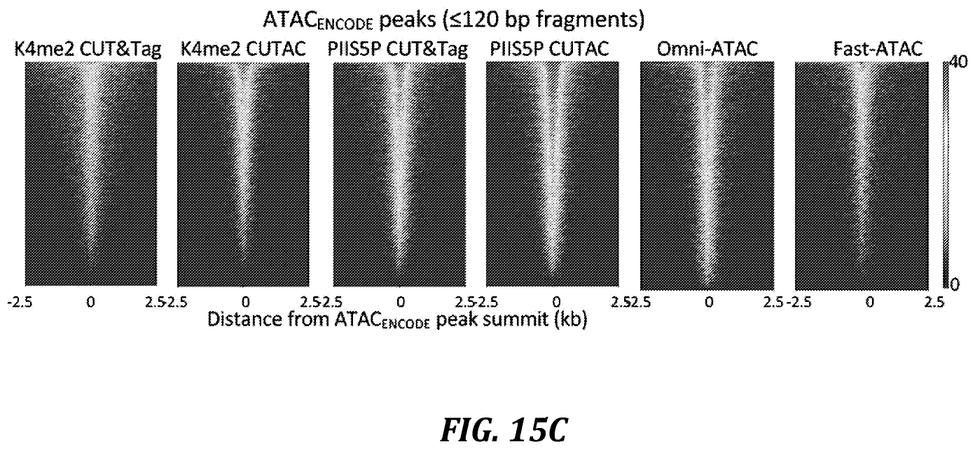

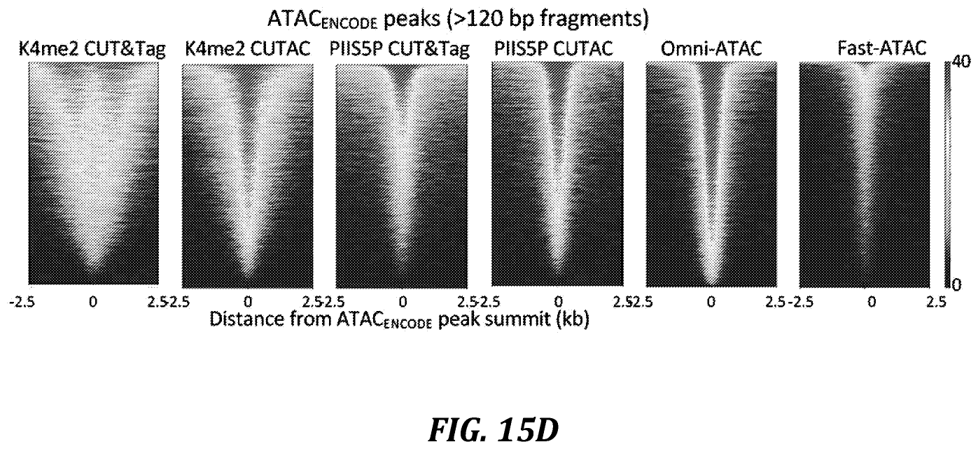

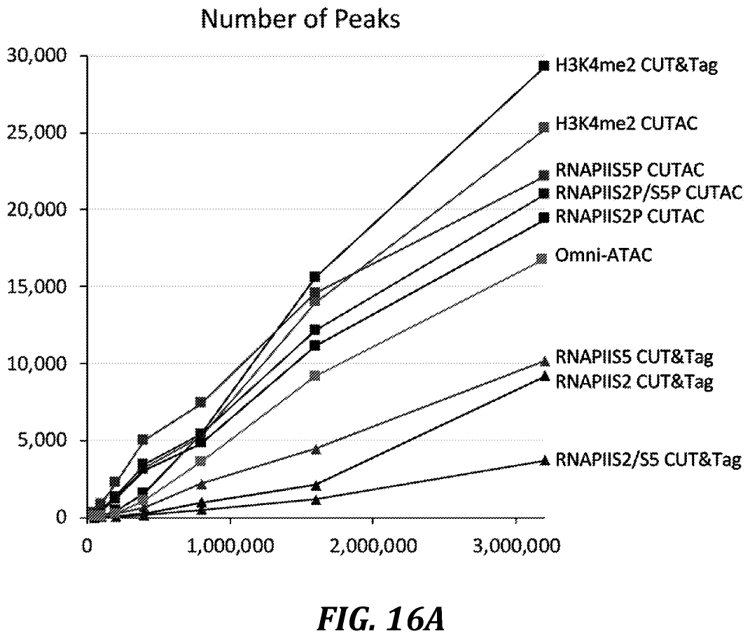

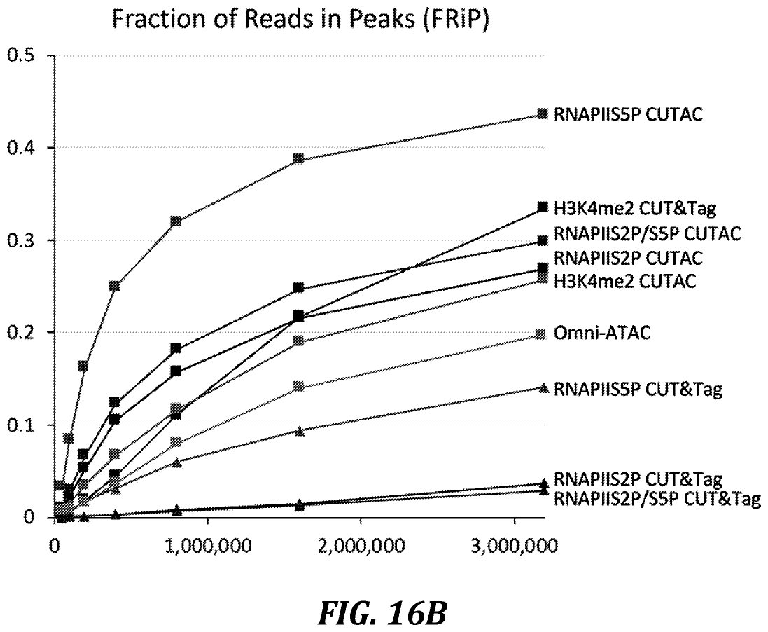

1. Align paired-end reads to hg19 using Bowtie2 version 2.3.4.3 with options: --end-to-end--very-sensitive--no-unal--no-mixed--no-discordant--phred33-I 10-X 700. For mapping E. coli carry-over fragments, the --no-overlap--no-dovetail options were also used to avoid possible cross-mapping of the experimental genome to that of the carry-over E. coli DNA that is used for calibration. Tracks are made as bedgraph files of normalized counts, which are the fraction of total counts at each basepair scaled by the size of the hg19 genome.
   Note: To calibrate samples in a series for samples done in parallel using the same antibody, counts of E. coli fragments carried over with the pA-Tn5 were used for an ordinary spike-in. The sample script in Github (“Henikoff/Cut-and-Run/blob/master/spike_in_calibration.csh”) can be used to calibrate based on either a spike-in or E. coli carry-over DNA.
2. The CUT&Tag Data Processing and Analysis Tutorial available in Protocols.io (“protocols.io/view/cut-amp-tag-data-processing-and-analysis-tutorial-bjk2kkye”) provides step-by-step guidance for mapping and analysis of CUT&Tag sequencing data. Most data analysis tools used for ChIP-seq data, such as bedtools (Quinlan laboratory, University of Utah), Picard (available as open-source under the MIT license), and deepTools (Ramirez, F. et al. (2016) deepTools2: A next Generation Web Server for Deep-Sequencing Data Analysis. Nucleic Acids Research, 44(W1):W160-W165), can be used on CUT&Tag data (FIGS. 14-16B). Analysis tools designed specifically for CUT&RUN/Tag data include the SEACR peak caller (Meers, M. P., et al. (2019) Peak calling by Sparse Enrichment Analysis for CUT&RUN chromatin profiling. Epigenetics & Chromatin 12, 42), also available as a public web server (at seacr.fredhutch.org), and CUT&RUNTools (Zhu, Q., et al. (2019) CUT&RUNTools: a flexible pipeline for CUT&RUN processing and footprint analysis. Genome Biol 20, 192).

### Recipes

1. Nuclei Extraction 1 (NE1) buffer
Mix 1 ml of 1M HEPES-KOH pH 7.9, 500 μl of 1 M KCl, 12.5 μl of 2 M spermidine, 500 μl of 10% Triton X-100, and 10 ml of glycerol in 38 ml dH₂O, and add 1 Roche Complete Protease Inhibitor EDTA-Free.

2. Wash buffer
Mix 1 ml of 1 M HEPES pH 7.5, 1.5 ml of 5 M NaCl, and 12.5 μl of 2 M spermidine, bring the final volume to 50 ml with dH₂O, and add 1 Roche Complete Protease Inhibitor EDTA-Free tablet. Store the buffer at 4° C. for up to 2 days.

3. Binding buffer
Mix 200 μl of 1M HEPES-KOH pH 7.9, 100 μl of 1 M KCl, 10 μl of 1M CaCl₂, and 10 μl 1 M MnCl₂, and bring the final volume to 10 ml with dH₂O. Store the buffer at 4° C. for up to several months.

4. Antibody buffer
Mix 5 μl of 200×BSA with 1 ml Wash buffer and chill on ice. BSA is present in some but not all antibody solutions, and 0.10% BSA in this buffer helps prevent bead loss during later steps.

5. 300-wash buffer
Mix 1 ml of 1 M HEPES pH 7.5, 3 ml of 5 M NaCl, and 12.5 μl of 2 M spermidine, bring the final volume to 50 ml with dH₂O, and add 1 Roche Complete Protease Inhibitor EDTA-Free tablet. Store at 4° C. for up to 2 days.

6. CUT&Tag Tagmentation buffer
Mix 1 ml of 300-wash buffer and 10 μl of 1 M MgCl₂ (to 10 mM).

7. CUTAC Tagmentation buffer
a. CUTAC-tag: Mix 197 μl of dH₂O, 2 μl of 1 M TAPS pH 8.5, and 1 μl of 1 M MgCl₂ (10 mM TAPS and 5 mM MgCl₂). Store the buffer at 4° C. for up to 1 day.
b. CUTAC-hex: Mix 97 μl of dH₂O, 100 μl of 20% (w/v) 1,6-hexanediol, 2 μl of 1 M TAPS pH 8.5, and 1 μl of 1 M MgCl₂(10 mM TAPS, 5 mM MgCl₂10% 1,6-hexanediol). Store the buffer at 4° C. for up to 1 day.

8. TAPS wash buffer
Mix 1 ml of dH₂O, 10 μl of 1 M TAPS pH 8.5, 0.4 μl of 0.5 M EDTA (10 mM TAPS, 0.2 mM EDTA).

9. 0.1% SDS Release solution
Mix 10 μl of 10% SDS and 10 μl of 1 M TAPS pH 8.5 in 1 ml of dH₂O

10. 0.67% Triton neutralization solution
Mix 67 μl of 10% Triton-X100+933 μl dH₂O

### Example 4

This example describes development of another illustrative embodiment of the disclosed CUTAC method, referred to as “CUT&Tag2for1”. In this embodiment, using the CUTAC protocol described above and mixing the anti-PolIIS5P antibody with an anti-H3K27me3 antibody, then computationally deconvolving the two signals, provides accurate mapping of both the active regulome (i.e., promoters and enhancers) and the silencing regulome (i.e., Polycomb domains). This CUT&Tag2for1 approach is particularly useful for single cell analysis.

### Abstract

Cleavage Under Targets & Tagmentation (CUT&Tag) is an antibody-directed strategy for in situ chromatin profiling in small samples and single cells. Examples 1 and 2 describe that activation of tethered Tn5 transposase under low-salt conditions (CUTAC) using antibodies that target promoters and enhancers produces high-resolution genome-wide chromatin accessibility maps. This Example describes a modified CUT&Tag protocol using a mixture of an antibody to the initiation form of RNA Polymerase II (Pol2 Serine-5 phosphate) and an antibody to repressive Polycomb domains (H3K27me3) followed by computational signal deconvolution to produce high-resolution maps of both the active and repressive regulomes in single cells. The ability to seamlessly map active promoters, enhancers and repressive regulatory elements using a single workflow provides a complete regulome profiling strategy suitable for high-throughput single-cell platforms

### Introduction

Throughout development, cells progress through a highly ordered series of cell fate transitions that gradually refine their cellular identities and direct their functional specializations. This “epigenetic” programming is controlled by gene expression networks that tune the production of RNA transcripts from the genome. In the transcriptionally repressed state, developmental genes display a characteristic broad distribution of the Polycomb Repressive Complexes-1 and -2 (PRC-1, and PRC-2), where PRC-2 tri-methylates histone H3 Lysine-27 (H3K27me3) that extends from upstream of the transcriptional start site (TSS) out across the gene body and beyond. During gene activation, cell type specific gene regulatory networks stimulate recruitment and firing of the RNA Polymerase II (Pol2) machinery and drive increased protein turnover and accessibility over transcriptional start sites (TSSs) and other cis-regulatory DNA elements that modulate gene expression (enhancers). During gene activation PRC-1 and PRC-2 are locally displaced and the H3K27me3 mark is lost. Defects in this interplay between active and repressive chromatin regulation underly a wide variety of human pathologies. However, because primary samples include complex mixtures of cells along various developmental trajectories, technologies that achieve single cell resolution are generally necessary to interrogate the molecular mechanisms that control gene expression in the normal and diseased states.

Single-cell genomic technologies that profile mRNAs (RNA-seq) or chromatin accessibility (ATAC-seq) can resolve the unique gene expression signatures and active regulatory features of distinct cell types from heterogenous samples. For single-cell profiling of the repressive chromatin landscape, single-cell H3K27me3 CUT&Tag is applied, wherein an antibody that targets H3K27me3 tethers a Protein A-Tn5 (pA-Tn5) fusion protein transposome complex to chromatin. To overcome the limitation of sparse or incomplete cellular profiles inherent to single cell genomics, droplet-based and nanowell platforms and combinatorial barcoding strategies dramatically increase the number of cells profiled in a single experiment. These sparse single cell profiles can then be grouped according to shared features to assemble more complete aggregate profiles of each cell type. Platforms that simplify the workflows and data analysis have greatly facilitated profiling the gene expression signatures and active and repressive chromatin landscapes of single cells.

To maximize genomic information from each single cell, several methods have been developed that simultaneously profile two or more modalities, such as accessible chromatin and mRNA or histone modifications and mRNA. Multi-modal single-cell profiling can resolve cell types that may be highly similar in the readout of one assay but show characteristic differences in the other and also allow direct comparisons between gene expression and components of the regulatory landscape in individual cells. Methods that simultaneously profile both the active and repressive epigenome could provide a more comprehensive understanding of cell fate regulation than can be obtained by profiling the active or repressive chromatin landscapes in isolation. However, multimodal methods require complex workflows and present data integration challenges, and there are no published methods that simultaneously profile the active and repressive chromatin landscape using a single workflow and readout modality.

As described above in Examples 1 and 2, a modified version of CUT&Tag has been developed where pA-Tn5 or Protein A/G-Tn5 (pAG-Tn5) is tethered near active TSSs and enhancers and tagmentation is performed under low salt conditions (referred to as CUTAC). Low-salt tagmentation results in highly specific integration of tethered Tn5 s within narrow accessible site windows to release chromatin fragments from active regulatory elements across the genome. In this example, CUTAC is extended to simultaneously profile regions of active and repressive chromatin within single cells by mixing antibodies that target both the initiating form of RNA Polymerase II and H3K27me3, followed by in silico deconvolution of the two epitopes. The disclosed deconvolution strategy leverages both the different tagmentation densities and the different fragment sizes to separate active and repressive chromatin regions directly from the data without reference to external information. In this way, CUT&Tag2for1 profiles both chromatin states using a single sequencing readout. As the workflow is similar to that of standard CUT&Tag, the method can be readily adopted for platforms already engineered for single-cell CUT&Tag.

### Results

Pol2S5p-CUTAC Maps Accessibility of Promoters and Functional Enhancers.

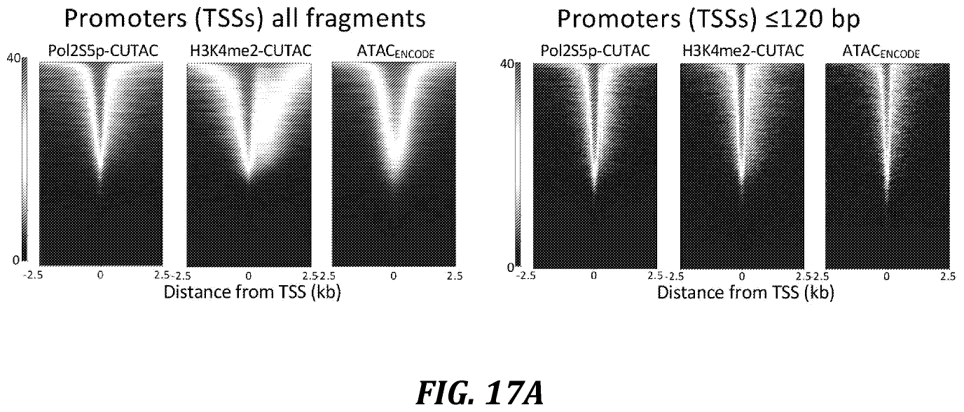

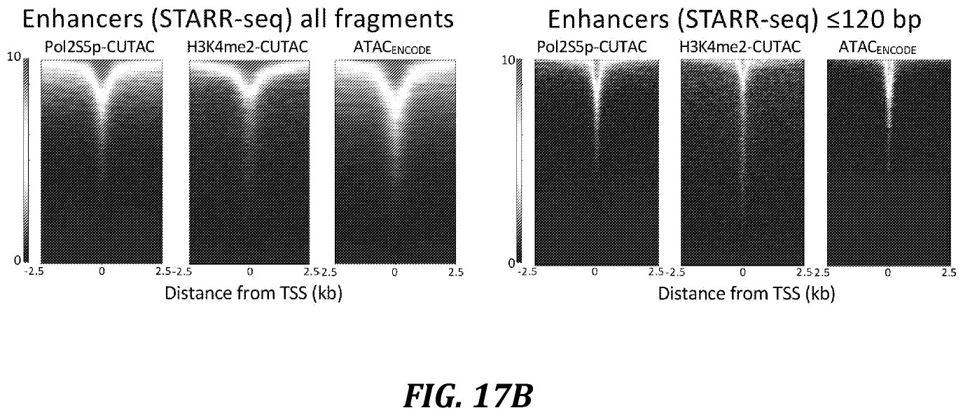

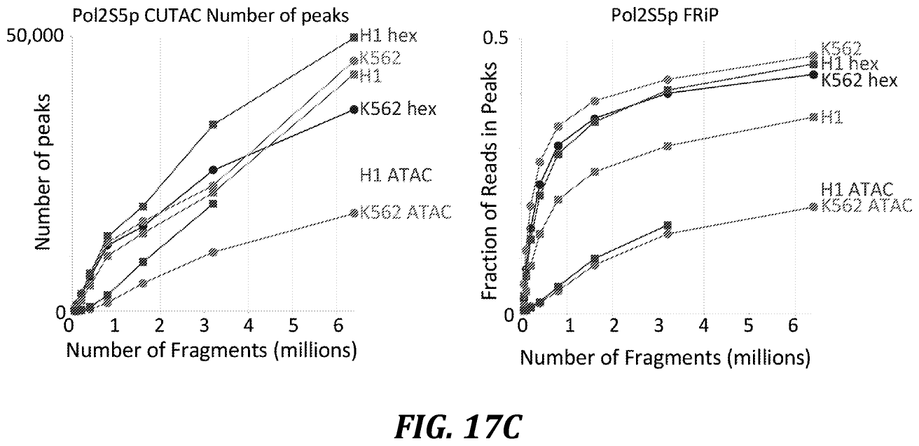

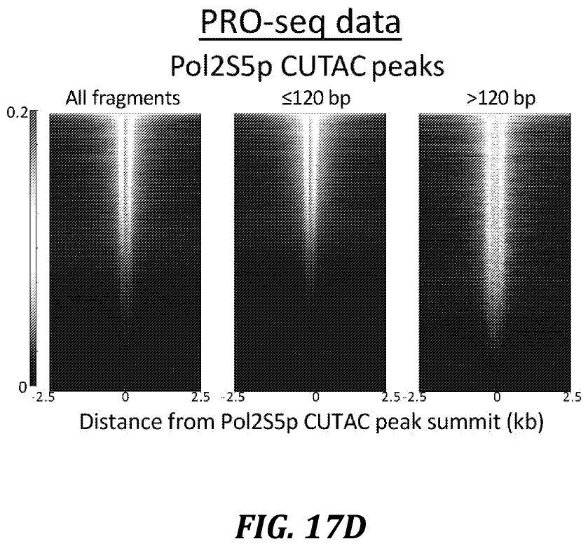

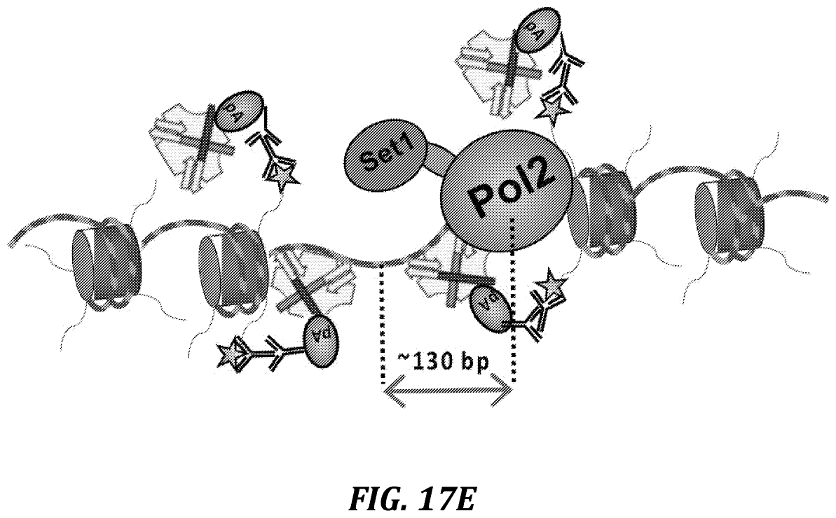

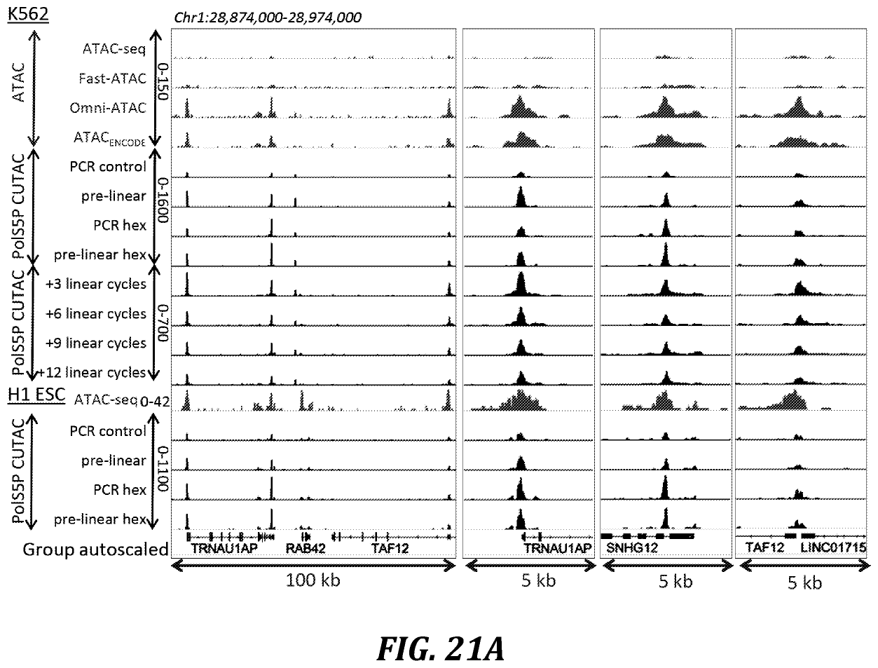

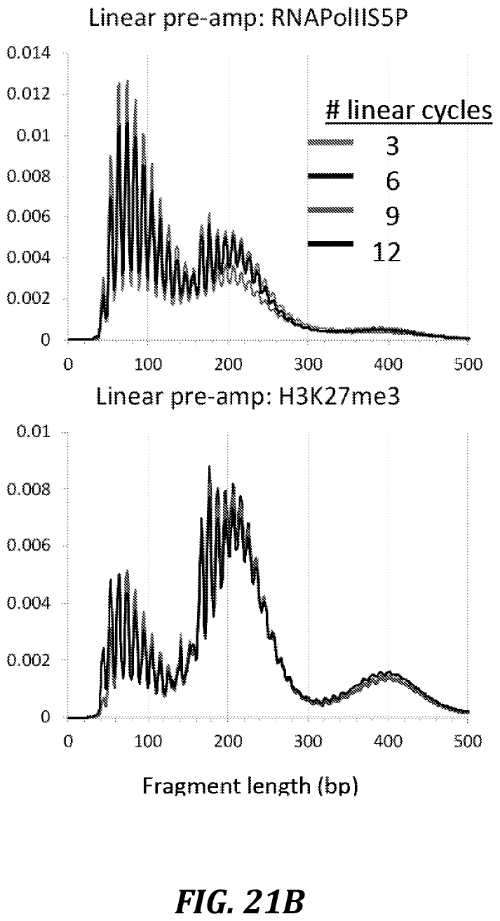

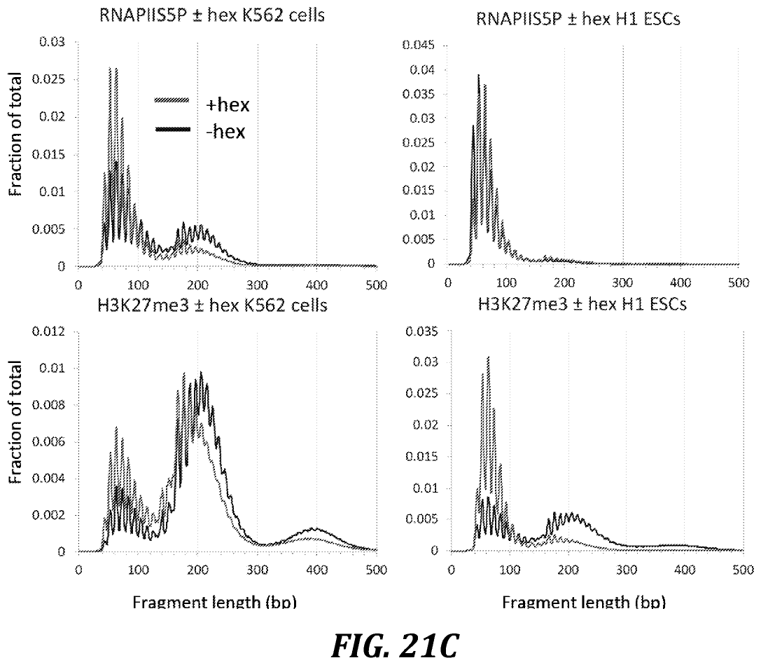

In CUTAC chromatin accessibility mapping, pA-Tn5 is tethered to active TSSs and enhancers using antibodies targeting either Histone-3 Lysine-4 dimethylation (H3K4me2) or trimethylation (H3K4me3) (Examples 1 and 2). It was reasoned that directly tethering pA-Tn5 to the initiating form of Pol2 (Pol2S5p), which is paused just downstream of the promoter, might also tagment accessible DNA under CUTAC conditions. Indeed, it was found that Pol2S5p CUTAC profiles display similar enrichment to H3K4me2 CUTAC at a variety of accessibility-associated features, including annotated promoters (FIG. 17A, left) and STARR-seq functional enhancers (FIG. 17B, left) in K562 Chronic Myelogenous Leukemia cells. Pol2S5p CUTAC yielded profiles with sharp peak definition and low backgrounds relative to high-quality ATAC-seq profiles (FIG. 21A). Genome-wide, high sensitivity and excellent signal-to-noise for Pol2S5p was observed, with more peaks called and higher Fraction of Reads in Peaks (FRiP) scores (Landt et al., (2012) Chip-seq guidelines and practices of the encode and modencode consortia. Genome Res. 22: 1813-1831) when plotted as a function of fragment number (FIG. 17C). Notably, restricting CUTAC fragments to those shorter than 120 bp further improved the resolution of accessible features (FIGS. 17A and 17B right), consistent with efficient Tn5 footprinting in exposed DNA. This interpretation is supported by aligning reads from PRO-seq, a transcriptional run-on method that precisely maps the position of the Pol2 active site (FIG. 17D), which shows it to be centered on average ˜130 bp from the accessibility footprints genome-wide (FIG. 17E). The close correspondence of both H3K4me2 CUTAC and Pol2S5p CUTAC to ATAC-seq provides direct evidence for the involvement of Pol2 in driving H3K4 methylation and chromatin accessibility. The fact that most promoters and STARR-seq enhancers are immediately adjacent to the paused initiating form of Pol2 is consistent with the suggestion that enhancers and promoters share the same chromatin configuration (Andersson et al., (2015). A unified architecture of transcriptional regulatory elements. Trends Genet. 31: 426-433).

CUT&Tag2for1 Distinguishes Active Versus Repressed Chromatin Based on Fragment Size.

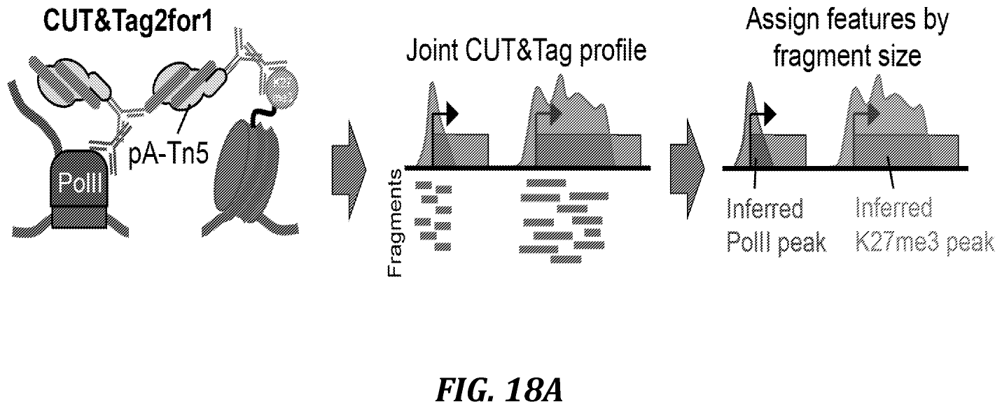

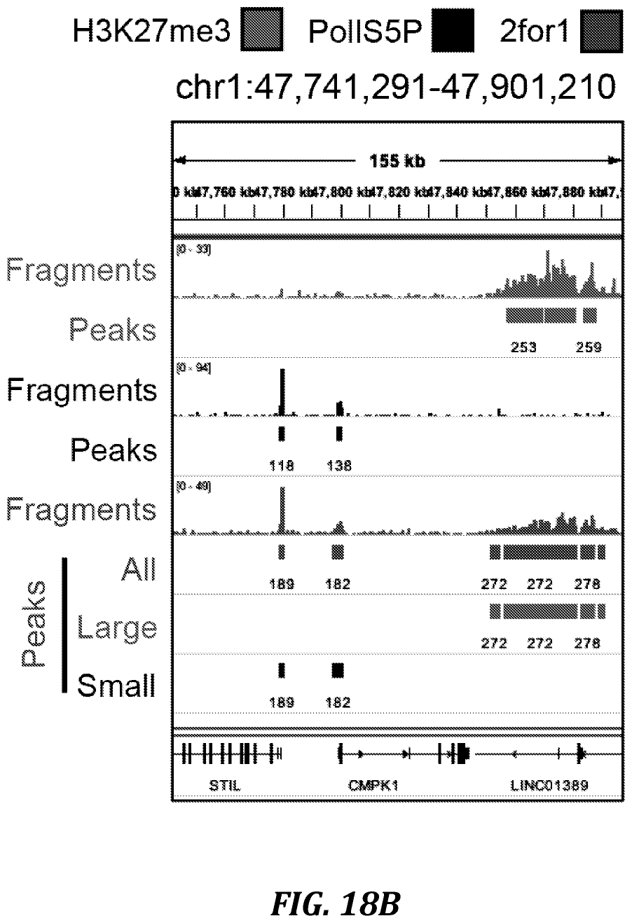

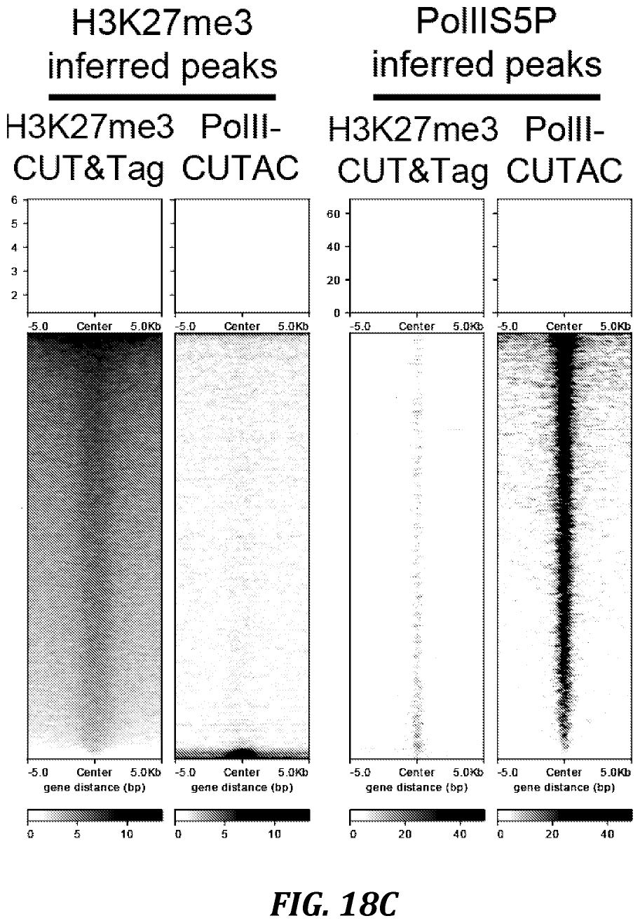

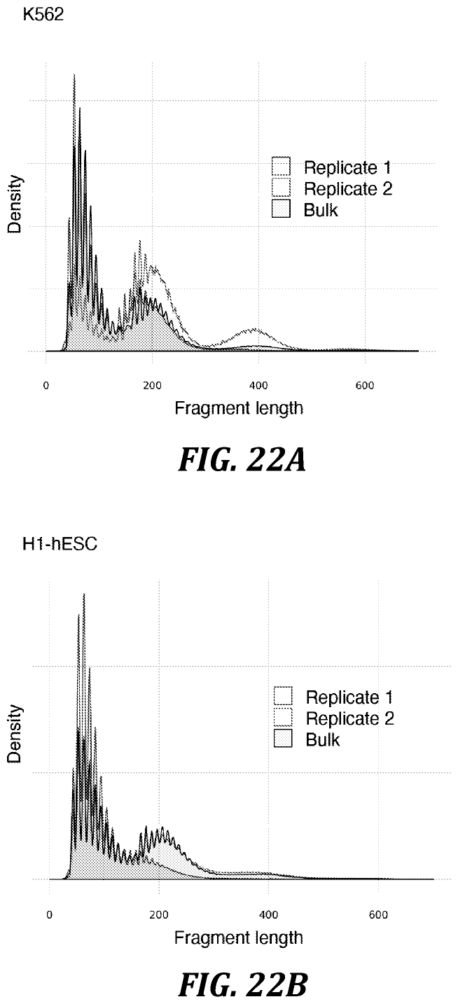

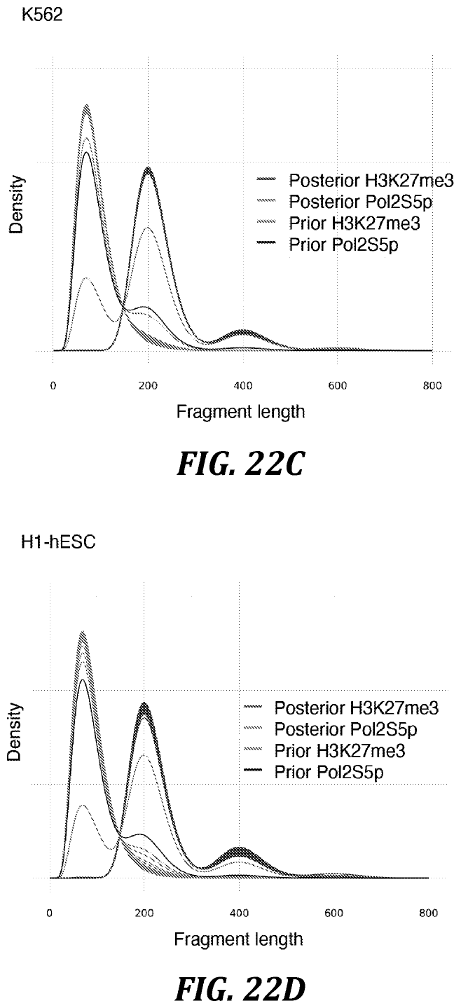

In comparison to H3K27me3 CUT&Tag profiles of the developmentally repressed chromatin landscape, it was noted that the CUTAC profiles include a much larger proportion of fragments that are <120 bp in both K562 and H1 embryonic stem cells (FIG. 22B). No consistent changes in fragment sizes were seen when 3-12 rounds of linear amplifications preceded PCR to minimize the competitive advantage of small fragments during the short PCR cycles used for CUT&Tag. However, including the polar organic compound 1,6-hexanediol during tagmentation resulted in a smaller fragment size distribution (FIG. 22C), with H1 cells showing a more marked effect than K562 cells, consistent with the previous finding that this increases penetrability of pAG-Tn5 and with “hyperdynamic” chromatin characteristic of embryonic stem cells. It was reasoned that this difference in fragment size might provide a general strategy to separate active and repressed chromatin profiles using a single sequencing readout from the same cells. Accordingly, Pol2S5p and H3K27me3 antibodies were mixed and the CUT&Tag protocol was followed for K562 and H1 samples with tagmentation under low-salt CUTAC conditions (FIG. 18A). It was found that when compared to individual CUTAC and H3K27me3 CUT&Tag profiles, features from both targets were well-represented in CUT&Tag2for1 profiles (FIG. 18B). A two-component Gaussian Mixture Model to the distribution of fragment size averages using an Expectation Maximization algorithm (Benaglia et al., (2010). Mixtools: An r package for analyzing mixture models. J Statistical Software 32(06)) was applied to separate peaks into inferred Pol2S5p-CUTAC (small fragment average) and H3K27me3 (large fragment average) profiles from the mixture. H3K27me3 CUT&Tag and CUTAC were found to map nearly exclusively to their fragment size-inferred peak sets (FIG. 18C), supporting the use of fragment size as an accurate feature classifier of CUT&Tag2for1 data. These data demonstrate that active and repressive chromatin features can be deconvolved in a joint assay with minimal additional effort.

CUT&Tag2for1 for Single Cells.

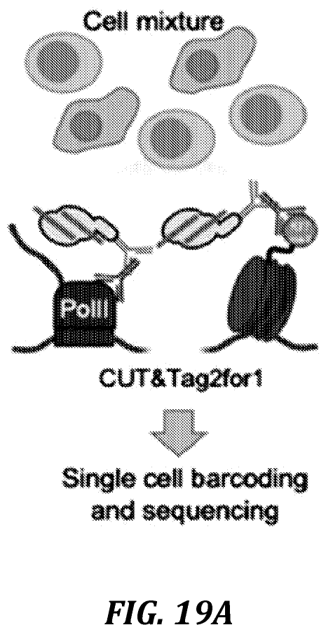

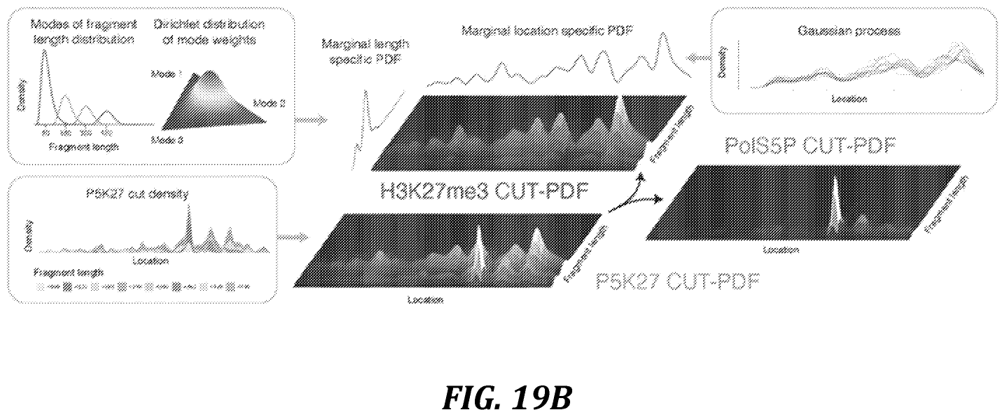

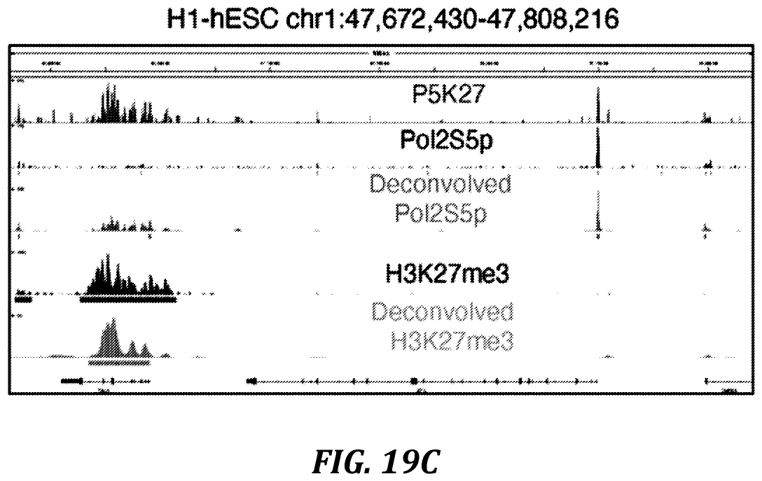

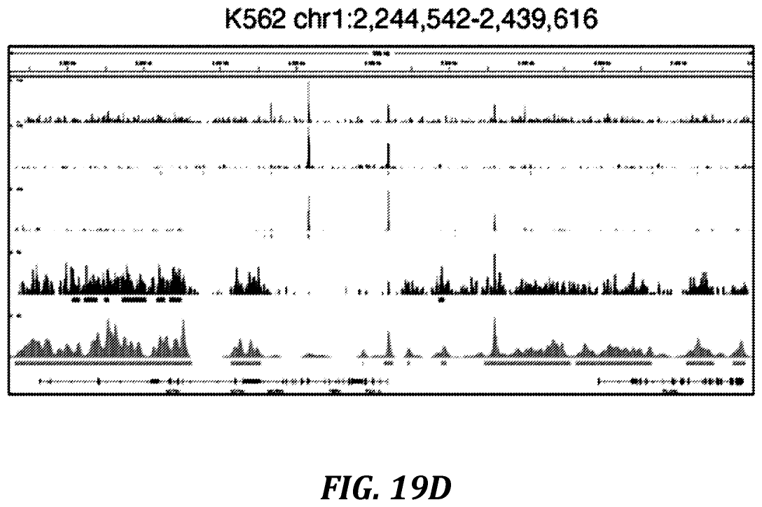

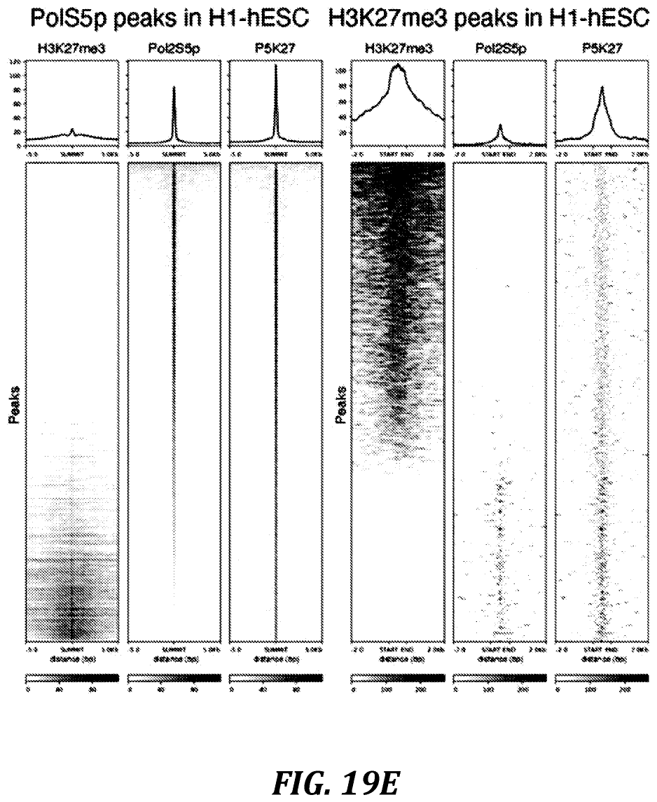

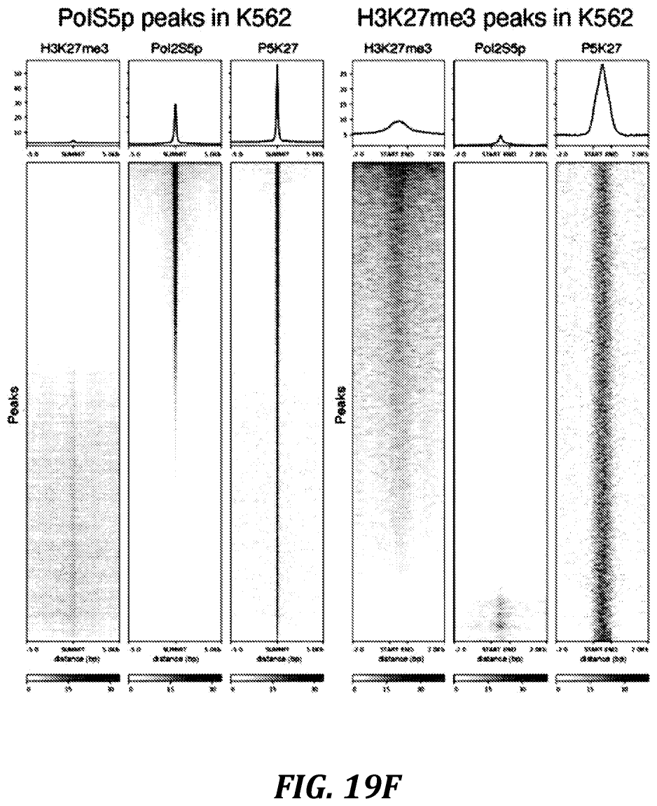

Given the successful adaptation of CUT&Tag for single cell profiling, it was next asked whether CUT&Tag2for1 could be adapted for single-cell chromatin characterization. CUT&Tag2for1 was performed in parallel for K562 and H1 cells, isolated single cells from the bulk mixtures on a Takara ICELL8 microfluidic device, and then amplified tagmented DNA with cell-specific barcodes (FIG. 19A). Because the fragment size distributions of the two targets can exhibit considerable overlap (FIG. 22A-22B), it was reasoned that deconvolution can be further enhanced by considering dependencies between positionally close adapter integration sites in the genome. i.e., observation of many cut sites from a particular target makes it more likely that an integration close to this set was induced from the same target feature. In addition, the differences in feature and peak width for the two epitopes (Pol2S5p peaks are narrow and sharp; H3K27me3 peaks are broad and diffuse) can also help the deconvolution. Thus, a novel deconvolution approach, “2for1separator”, was developed using Bayesian statistics to model the CUT&Tag2for1 signal as a mixture of Pol2S5p and H3K27me3 signals by considering fragment length distributions, positional dependencies, and feature widths of the two targets (FIG. 19B, Methods). The fragment length distribution is encoded as a mixture of log-normal distributions over the characteristic modes of chromatin data, and the neighborhood information i.e., positional dependencies and feature widths are modeled using a Gaussian process (FIG. 19B). The deconvolved signals were then used as inputs to a peak-calling procedure to identify Pol2S5p and H3K27me3 peaks from CUT&Tag2for1 data.

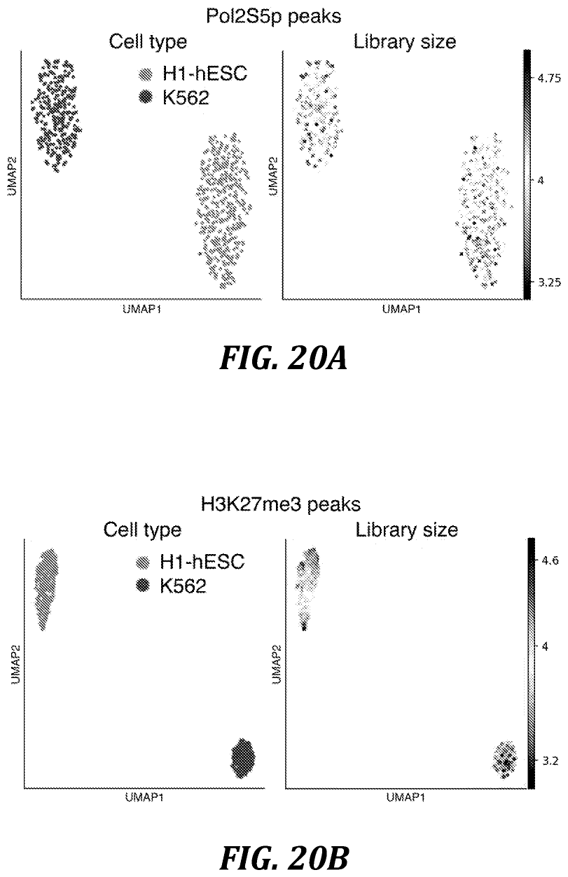

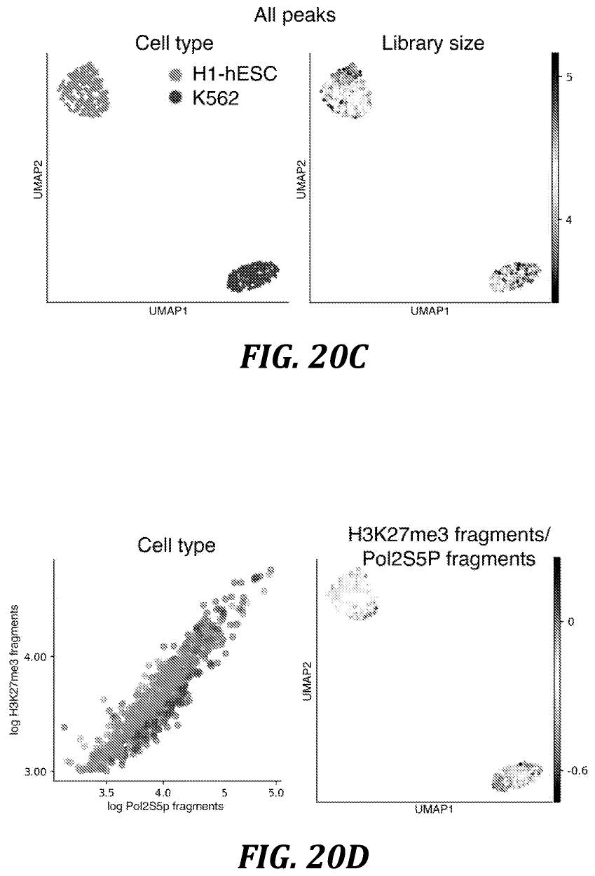

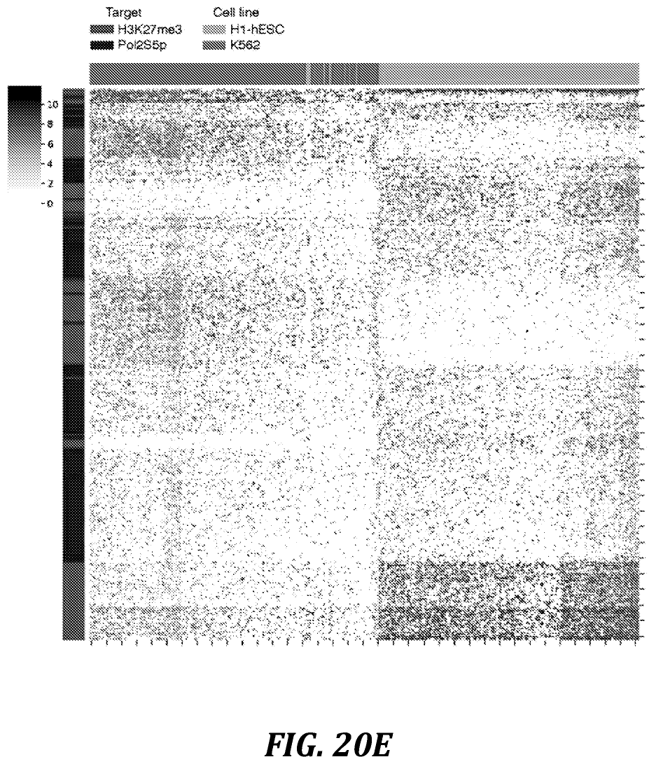

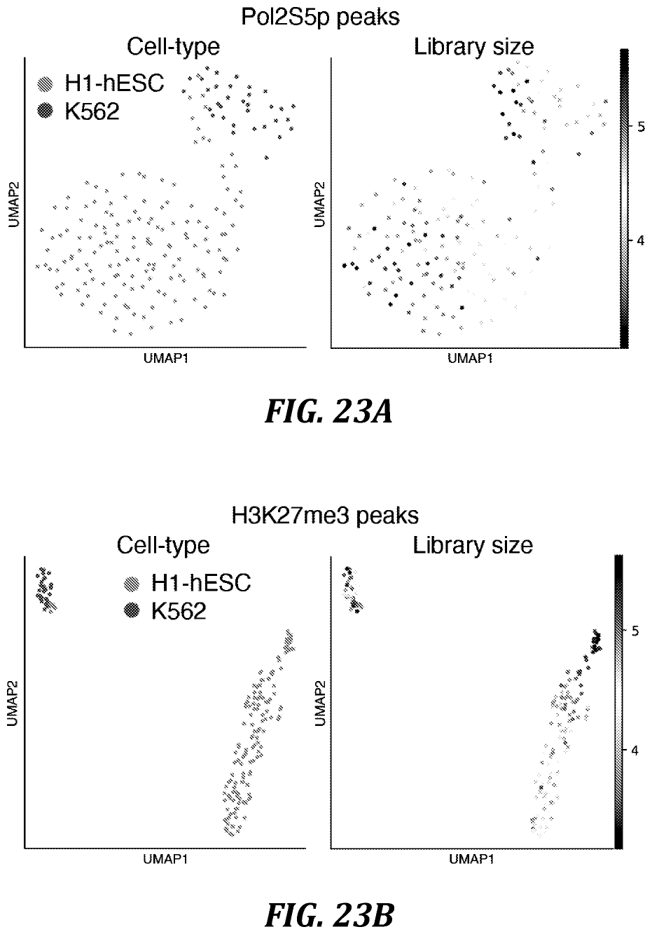

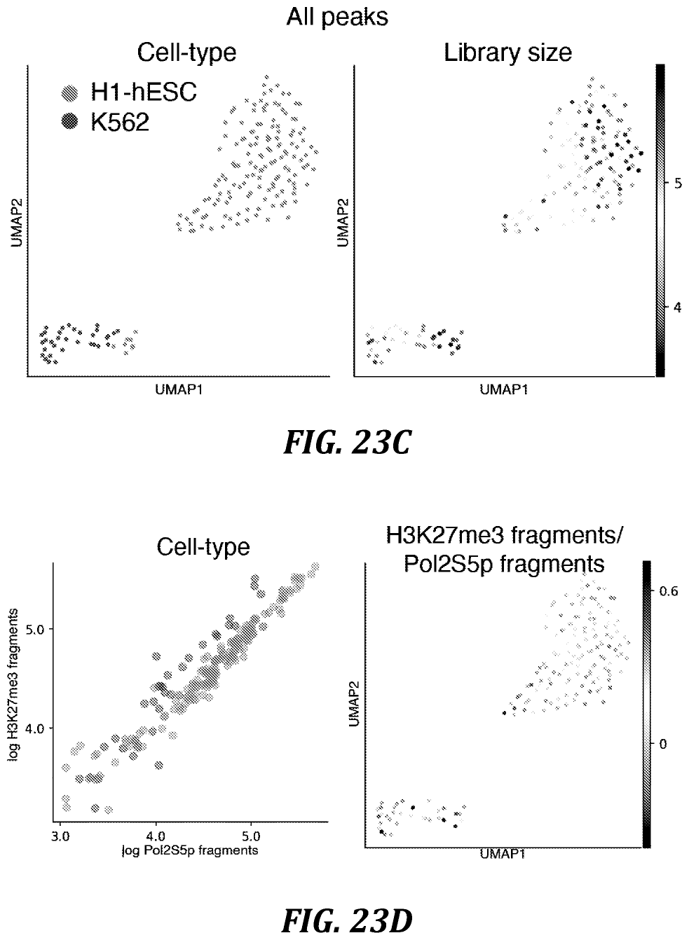

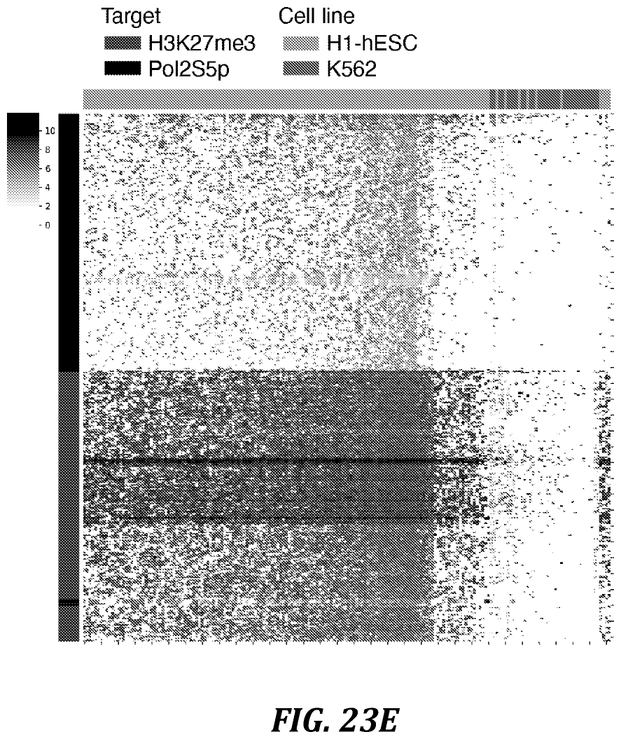

Applied to K562 and H1 CUT&Tag2for1 single-cell data, the disclosed 2for1separator algorithm accurately determined Pol2S5p and H3K27me3 peaks, showing strong enrichment of the correct single antibody signals in the respective peaks (FIGS. 19C-19F). Single-cell data was then visualized using UMAP projections of feature counts and it was observed that cells from the two lines can be near-perfectly distinguished based on Pol2S5p peaks (FIGS. 20A and 23A), H3K27me3 peaks (FIGS. 20B and 23B), or the combination of the two (FIGS. 20C and 23C). The number of fragments mapping to Pol2S5p and H3K27me3 peaks were compared and a strong correlation in both cell types was observed (FIG. 20D, correlation: 0.95), with an even balance of fragments between the two targets in individual cells. In line with this observation, the 400 most variable Pol2S5p peaks and H3K27me3 peaks were sufficient to distinguish the two cell types (FIG. 20E), demonstrating that CUT&Tag2for1 can be used to identify both active and repressive chromatin features in the same single cells, and they can be used coordinately to distinguish cell identity.

### Discussion

Single cell genomics methods for profiling the transcriptome, proteome, methylome and accessible chromatin landscape have advanced rapidly in recent years. Currently, approaches for profiling single epigenome targets on their own or in combination with other modalities (e.g. transcriptome) are the state of the art, but methods for simultaneously profiling the active versus repressive chromatin landscape in single cells are still lacking. CUT&Tag2for1 combines simple antibody mixing in a single workflow with a single sequencing readout to profile and computationally separate accessible and repressed chromatin regions. Single-cell CUT&Tag2for1 avoids the complex workflows, multi-level barcoding and apples-and-oranges integration challenges posed by multimodal profiling methods.

CUT&Tag2for1 was inspired by the observation that Pol2S5p CUTAC, developed based on the development of the H3K4me2 CUTAC method (see Examples 1 and 2), yields a different average fragment size profile than H3K27me3 CUT&Tag, and therefore that the two could be distinguished in a single assay. The DNA fragment length data dimension allows for a priori assignment of target origin, which is in keeping with the myriad advantages of using fragment length to elucidate fine grain chromatin structure. By also using feature width information in a probabilistic model, a robust separation of the active and repressive landscapes is obtained.

Single-cell CUT&Tag2for1 can assign Pol2S5p or H3K27me3 target origin with high fidelity in the absence of ground truth datasets. In principle this deconvolution strategy can be applied to other large fragment/large feature nucleosomal marks, such as H3K36me3 for active gene body chromatin, combined with small fragment/small feature non-nucleosomal proteins such as transcription factors. The relatively high abundance of both H3K27me3 and Pol2S5p and the fact that in combination they profile virtually the entire chromatin developmental regulatory landscape, renders the current implementation of CUT&Tag2for1 an attractive genomics-based strategy for a wide range of development and disease studies.

### Experimental Model and Subject Details

Human Cell Culture

Human female K562 Chronic Myleogenous Leukemia cells (ATCC) were authenticated for STR, sterility, human pathogenic virus testing, mycoplasma contamination, and viability at thaw. H1 (WA01) male human embryonic stem cells (hESCs) (WiCell) were authenticated for karyotype, STR, sterility, mycoplasma contamination, and viability at thaw. Cells were cultured as previously described (Janssens et al., (2018). Automated in situ chromatin profiling efficiently resolves cell types and gene regulatory programs. Epigenetics Chromatin 11: 74). Briefly, K562 cells were cultured in liquid suspension. H1 cells were cultured in Matrigel (Corning)-coated plates at 37° C. and 5% CO₂ using mTeSR-1 Basal Medium (STEMCELL Technologies) exchanged every 24 hours.

### Method Details

Bulk CUT&Tag, CUTAC and CUT&Tag2for1

CUTAC using Pol2S5p for accessible site mapping was performed as described in a step-by-step protocol(Henikoff et al., (2021). Simplified epigenome profiling using antibody-tethered tagmentation. bio-protocol 11: e4043). Briefly, cells were harvested by centrifugation, washed with PBS and nuclei prepared and lightly cross-linked (0.1% formaldehyde 2 min), then washed and resuspended in Wash buffer (10 mM HEPES pH 150 mM NaCl, 2 mM spermidine and Roche complete EDTA-free protease inhibitor), aliquoted with 10% DMSO and slow-frozen to −80° C. in Mr. Frosty containers (Sigma-Aldrich cat. no. C1562). CUT&Tag, CUTAC and CUT&Tag2for1 were performed in parallel in single 0.6 mL PCR tubes by mixing with Concanavalin A magnetic beads and performing incubation and wash steps on a magnet. Primary (anti-rabbit) antibody [1:50 for Pol2S5p (Cell Signaling Technology cat. no. 13523) or 1:100 for H3K27me3 (Cell Signaling Technology cat. no. 9733)] in Wash buffer+0.1% BSA was added and beads were incubated at room temperature for 1-2 hr or overnight at 4° C. For CUT&Tag2for1, primary antibodies were mixed in the same concentrations. Beads were magnetized and the supernatant was removed, then the beads were resuspended in guinea pig anti-rabbit secondary antibody (Antibodies Online cat. no. ABIN101961) and incubated 0.5-1 hr. Beads were magnetized, the supernatant was removed, then the beads were resuspended in pAG-Tn5 pre-loaded with mosaic-end adapters (Epicypher cat. no. 15-1117 1:20) in 300-wash buffer (Wash buffer except containing 300 mM NaCl) and incubated 1-2 hr at room temperature. Following magnetization, supernatant removal and washing in 300-wash buffer, the beads were incubated at 37° C. in either 10 mM MgCl₂, 300 mM NaCl (for CUT&Tag) for 1 hr or 5 mM MgCl₂, 10 mM TAPS pH 8.5 (for CUTAC and CUT&Tag2for1) for 10-30 min. In some experiments CUTAC and CUT&Tag2for1 tagmentation was performed in 5 mM MgCl₂, 10 mM TAPS pH 8.5 with addition of 10% (w/v) 1,6-hexanediol (Sigma-Aldrich cat. no. 240117-50G) or 10% (v/v) N,N-dimethylformamide (Henikoff S, et al., Efficient chromatin accessibility mapping in situ by nucleosome-tethered tagmentation. Elife 2020, 9:e63274). Bead suspensions were chilled on ice, magnetized, the supernatant was removed, then beads were washed with 10 mM TAPS pH 8.5, 0.2 mM EDTA, and resuspended in 5 μL 0.1% SDS, 10 mM TAPS pH 8.5. Beads were incubated at 58° C. in a thermocycler with heated lid for 1 hr, followed by addition of 15 μL 0.67% Triton X-100 to neutralize the SDS. Barcoded PCR primers were added followed by 25 μL of either NEBNext 2× Master Mix (ME541L, non-hotstart) or KAPA Polymerase 2× master mix [Roche KAPA HiFi plus dNTPs: 360 μL 5×KAPA HiFi buffer, 54 μL KAPA dNTP mix (10 mM each), 36 μL KAPA non-hotstart DNA Pol (1 U/μL), 450 μL dH₂O]. Gap-filling and 12-cycle PCR were performed: 58° C. 5 min, 72° C. 5 min, 98° C. 30 sec, 12 cycles of (98° C. 10 sec denaturation and 60° C. 10 sec annealing/extension), 72° C. 1 min, and 8° C. hold. In some experiments linear pre-amplification was performed using this program with 3-12 cycles but with only i5 primers, followed by addition of i7 primers at 8° C. and 10-12 cycles of (98° C. 10 sec denaturation and 60° C. 10 sec annealing/extension), then 72° C. 1 min, and 8° C. hold, and in other experiments the initial 98° C. denaturation step was extended from 30 sec to 5 min, but no consistent differences in the resulting libraries were observed. SPRI paramagnetic beads were added directly to the bead-cell slurry for clean-up as described by the manufacturer (Magbio Genomics, cat. no. AC-60500). Elution was in 20 μL 1 mM Tris pH 8.0, 0.1 mM EDTA. Library quality and concentration were evaluated by Agilent Tapestation capillary gel analysis, barcoded libraries were mixed and PE25 sequencing performed on an Illumina HiSeq2500 by the Fred Hutch Genomics Shared Resource.

Single-Cell CUT&Tag2for1

CUT&Tag2for1 was performed using lightly fixed K562 and H1 nuclei. Frozen nuclei were thawed and aliquots containing 20,000 nuclei were centrifuged at 700×g for 4 minutes at 4° C. Nuclei were washed once with Wash buffer, centrifuged again, and then resuspended in Antibody buffer (10 mM HEPES pH 150 mM NaCl, 2 mM spermidine, 2 mM EDTA, 0.1% BSA, and Roche complete EDTA-free protease inhibitor) with primary anti-Pol2S5p antibody (Cell Signaling Technology cat. no. 13523, 1:50) and anti-H3K27me3 (Cell Signaling Technology cat. no. 9733, 1:100) in 0.6 mL PCR tubes. Primary antibody binding was performed overnight at 4° C. Samples were centrifuged at 700×g for 4 minutes at 4° C. between incubation steps and incubated for 1 hour at room temperature for the guinea pig anti-rabbit secondary antibody (Antibodies Online cat. no. ABIN101961 1:100) and for 1 hour at room temperature for pAG-Tn5 (Epicypher cat. no. 15-1117, 1:20) tethering. Samples were then centrifuged, washed with 300-wash buffer, pelleted by centrifugation, and then resuspended in 5 mM MgCl₂, 10% hexanediol, 10 mM TAPS pH 8.5 for 20 min at 37° C. for tagmentation. Reactions were stopped by adding EDTA to a final concentration of 1 mM, and kept at 4° C. until dispensation on the ICELL8 platform.

Cells were processed on the ICELL8 instrument according to a previously optimized protocol for release of tagmented DNA in SDS, followed by a Triton X-100 neutralization step and PCR amplification. Briefly, the volume of 10 mM TAPS Buffer pH 8.5 was adjusted to 65 μL per 20,000 nuclei to yield a concentration of ˜300 nuclei/μL and nuclei were stained with 1×DAPI and 1× secondary diluent reagent (Takara Cat #640196). The 8 source wells of the ICELL8 were loaded with 65 μL of the suspension of tagmented nuclei and dispensed into a SMARTer ICELL8 350v chip (Takara Bio, cat. no. 640019) at 35 nL per well. The chip was then sealed for imaging and spun down at 1200×g for 1 min. Imaging on a DAPI channel confirmed the presence of single cells in specific wells. Non-single-cell wells were excluded from downstream reagent dispenses. A volume of 35 nL of 0.19% SDS in 10 mM TAPS Buffer pH 8.5 was dispensed into active wells and the chip was dried, sealed and spun down at 1200×g for 1 min. The chip was placed in a thermocycler and held at 58° C. for 1 hr to release tagmented chromatin. The chip was spun at 1200×g for 1 min before opening, and 35 nL of 2.5% Triton X-100 in distilled deionized H₂O was dispensed into all active wells. To index the whole chip, 72×72 i5/i7 primers containing unique indices (5,184 microwells total) were dispensed at 35 nL in wells that contained single cells, followed by two dispenses of 50 nL (100 nL total) KAPA PCR mix (2.775×HiFi Buffer, 0.85 mM dNTPs, 0.05 U KAPA HiFi polymerase/μL, Roche Cat #07958846001). The chip was sealed for heated incubation and spun down at 1200×g for 1 min after each dispense. PCR on the chip was performed with the following protocol: 5 min at 58° C., 10 min at 72° C. and 2 min at 98° C., followed by 15 cycles of 15 s at 98° C., 15 s at 60° C. and 10 s at 72° C., with a final extension at 72° C. for 2 min. The contents of the chip were then centrifuged into a collection tube (Takara Cat #640048) at 1200 g for 3 min. Two rounds of SPRI bead cleanup at a 1.3: 1 v/v ratio of beads to sample were performed to remove residual PCR primers and detergent. Samples were resuspended in 20 μL of 10 mM Tris-HCl pH 8.0. Library quality and concentration were evaluated by Agilent Tapestation capillary gel analysis, and single-cell CUT&Tag2for1 samples were then pooled with bulk libraries prepared using compatible barcodes. PE25 sequencing was performed on an Illumina HiSeq2500 by the Fred Hutch Genomics Shared Resource.

Deconvolution Using Fragment Size (Bulk)

Peaks were called using SEACR v1.3 (Meers M P, et al. Peak calling by Sparse Enrichment Analysis for CUT&RUN chromatin profiling. Epigenetics Chromatin 2019, 12:42). Fragments overlapping peaks were ascertained using bedtools intersect (Meers M P, et al. Peak calling by Sparse Enrichment Analysis for CUT&RUN chromatin profiling. Epigenetics Chromatin 2019, 12:42). For each peak, the average fragment size of all fragments overlapping the peak in question were calculated and then fit the distribution of average fragment sizes across all peaks to a mixture of two Gaussian distributions using Mixtools NormalMixEM (Benaglia et al., (2010). Mixtools: An r package for analyzing mixture models. J Statistical Software 32). Peaks were partitioned into “large” (H3K27me3) and “small” (PolIIS5P) fragment size classes based on the average fragment size threshold at which the two calculated Gaussian distributions intersect. Bulk H3K27me3 CUT&Tag and PolIIS5P CUTAC were mapped onto large and small peak classes in heatmap form using Deeptools (Ramirez et al., (2016). Deeptools2: A next generation web server for deep-sequencing data analysis. Nucleic Acids Res 44: W160-165).

Deconvolution Using Feature Width and Fragment Size (Single-Cell)

CUT&Tag fragments result from two independent integration events resulting in two tagmentation cut sites after gap-filling, barcoded PCR and DNA sequencing. Rather than trying to attribute each fragment to either Pol2S5p or H3K27me3, the deconvolution approach (2for1separator algorithm) estimates how likely was a cut derived from Pol2S5p or H3K27me3 antibodies. Three key insights were used for the deconvolution: (i) fragment length distributions are significantly different between the two targets (FIGS. 21A-21C), (ii) cuts from a target have a positional dependency i.e., observation of multiple cuts from a specific target at a genomic location most likely means a cut close to this set was also induced by the same target, and (iii) feature widths between the two targets are typically different. Pol2S5p peaks are narrow and sharp, whereas H3K27me3 domains are broad and diffuse (FIG. 18B). Motivated by this, the 2for1separator algorithm was developed to deconvolve the CUT&Tag2for1 data into two signal tracks representing the density of chromatin cut sites targeted by H3K27me3 and PolS5p antibodies, respectively. The deconvolved signals were then used to identify narrow Pol2S5p peaks and broad H3K27me3 domains in the data.

Overview of 2for1Separator

Formally, a cut is represented as a tuple (x, l) where x stands for the location in the genome and l the length of the fragment it belongs to. The density of CUT&Tag2for1 cuts at cut-site x with fragment length, l can be represented as

ƒ_{2for1}(x,l)=λ_{H3K27me3}ƒ_{H3K27me3}(x,l)+λ_{Pol2S5P}ƒ_{Pol2S5P}(x,l) (1)

where function ƒ is the probability density function (PDF). λ_{H3K27me3} and λ_{Pol2S5P} represent the respective weights.

It is assumed that the length l and position x are independently distributed for each target, therefore ƒ_{H3K27me3} (x, l) can be written as

ƒ_{H3K27me3}(x,l)=h_{H3K27me3}(x)·h_{H3K27me3}(l) (2)

Similarly, for Pol2S5p

ƒ_{Pol2S5P}(x,l)=h_{Pol2S5P}(x)·h_{Pol2S5P}(l) (3)

where h(x) is the location-specific marginal cut-side probability density function and h(l) is the location-independent marginal fragment length probability density function.

Fragment Length Distribution Prior

The fragment length marginal PDFs, h_{Pol2S5P}(l) and h_{H3K27me3}(l), are parameterized separately to account for the differences in length distributions between the two targets. Length distributions show characteristic modes irrespective of the target (FIGS. 21A-22B). Thus, the fragment length PDF is represented as a mixture of four log-normal distributions with modes centered at 70, 200, 400, 600 (FIG. 19B). A distinction is not made for fragments that are >800 base pairs in length since they occur rarely. It is assumed the weights of the modes to follow a Dirichlet distribution—effective for modeling multinomial distributions, that were roughly based on the single antibody data.

Through a rough estimate of these mode weights and with arguable uncertainty of the true distribution the Dirichlet-parameter vector (450, 100, 10, 1) was ultimately used for Pol2S5p and (150, 300, 50, 10) for H3K27me3. Note that the deconvolution inferred weights remain very consistent across multiple fragment subsamples, while deviating strongly from the mean of the Dirichlet prior (FIGS. 22C and 22D), indicating that the result is data driven and not very sensitive to the exact choice of prior parameters. One only needs to encode the fact that Pol2S5p are shorter on average than H3K27me3 peaks fragments.

Cut-Site Densities and Prior

The cut-site PDFs were modeled as Gaussian Processes (GP), a powerful technique that can accurately infer the shape of the signal by considering the positional dependencies in signal values (FIG. 19B). The GP is used to predict the log cut density at a particular cut-site as a function of all the cuts in the neighborhood. A GP is defined by mean and covariance functions where the covariance function encodes the neighborhood information, i.e., positional dependencies between cuts and feature widths, making GPs ideally suited to infer cut-site density functions for the two targets.

An empirical approach was taken to define the covariance function of the Gaussian process. The Gaussian kernel density estimates (σ=200) of cuts from the H3K27me3 CUT&Tag and Pol2S5p-CUTAC experiments were examined. It was determined that the autocorrelation of the log-density, representing both local dependencies, is well approximated through the Matern covariance function (nu=3/2) (Genton M G. Classes of kernels for machine learning: a statistics perspective. J Mach Learn Res 2002, 2:299-312). Based on the observed autocorrelations, this covariance function was chosen with length scales 500 and 2000 as kernels of the GP for the Pol2S5p and H3K27me3 targets respectively to account for feature width differences. It is noted that differences in feature widths is not a necessary component, and the disclosed model can deconvolve the signals as long as the fragment length distributions between the two targets are different.

Constraints on the Gaussian Process

The functions generated through the GP express the desired smoothness and mean value but are not guaranteed to represent probability density functions. To ensure that the generated functions indeed represent PDFs, two additional constraints must be guaranteed: (i) strict positivity (ii) a fixed integral, without which, the resulting likelihood could grow infinitely jeopardizing any posterior estimate of the location specific PDFs.

Positivity is ensured by applying the exponential. The cut-site PDF h_{Pol2S5P}(x) was model as

λ_{Pol2S5P}h_{Pol2S5P}(x)=exp(g_{Pol2S5P}(x)) (4)

where g_{Pol2S5P} is a random variable of a GP. Similarly,

λ_{H3K27me3}fh_{H3K27me3}(x)=exp(g_{H3K27me3}(x)) (5)

The sum of the two PDFs in Equations (4) and (5) should integrate to one for a fixed integral. Rather than constraining the integral to one, we aim for a density function that integrates to the total number of observed cuts for ease of implementation. This representation results in a constant factor in the combined likelihood function and does not impact the inference. As an added benefit of this formulation, the inferred density function has the unit “cuts per base pair” and hence is insensitive to the size of the deconvolved genomic region. Further, this also results in the log-density to have an approximate mean value of 0 across the whole genome and, thus, a zero-mean GP is used. This integral is approximated with the rectangle rule, by assuming one rectangle per cut site and a width such that neighboring rectangles touch at the midpoint between the cut sites. To enforce the correct integral, a log-normal distribution of the resulting approximation is imposed around the desired value and a very small standard deviation of 0.001, since enforcing a constraint to a fixed value makes the inference intractable.

Inference

To infer the most likely target specific chromatin cut PDF, the gradient descent method we use, limited-memory BFGS on the posterior parameter distribution, to find the local maximum a posteriori point (MAP). The MAP represents the most likely cut PDFs and fragment length distributions in the chosen parametrization of the model.

Pol2S5p Peak Calling

Deconvolved Pol2S5p signals were used to perform peak calling. Each region containing cuts with deconvolved Pol2S5p signal greater than a computed threshold as Pol2S5p peaks were nominated. Pol2S5p peaks longer 100 bases are retained for downstream analysis. The position is identified within the peak with maximal deconvolved signal as the summit.

First, the fraction of cuts that are derived from Pol2S5p to compute the threshold are estimated. The fraction, denoted as \hat{r}_{Pol}, is estimated as the ratio between the integral of Pol2S5p deconvolved density and the integral of the combined density. In practice, it was found this estimate to be susceptible to instability and therefore a beta distribution with parameters α=0.5, β=0.5 was used as a prior to derive a robust estimate. With a further conservative assumption that 50% of the Pol2S5p cuts fall into Pol2S5p reproducible peaks, the expected value of the fraction of cuts that fall in Pol2S5p peaks is

$${r}_{Pol} = \frac{1 + 2 {\hat{r}}_{Pol}}{8}$$

Therefore, the r_{Pol}th percentile of the deconvolved signal value was used as the threshold i.e., regions with cuts with deconvolved signal higher than the r_{Pol}th percentile are identified as Pol2S5p peaks.

H3K27me3 Domains

A procedure analogous to Pol2S5p peak calling was used to identify H3K27me3 domains using the deconvolved H3K27me3 signal. It was observed that large H3K27me3 domains appear as discontinuous signal blocks (FIG. 19C, right panel). An additional smoothing was therefore applied on the deconvolved H3K27me3 signal using a Gaussian filter and computed the average between the smoothed and original signal. The peak calling procedure was then repeated on the smoothed signal and H3K27me3 domains were identified as the union of domains identified using deconvolved and the additionally smoothed signals. Only peaks wider than 400 bases are retained for downstream analysis.

Overlap Peaks

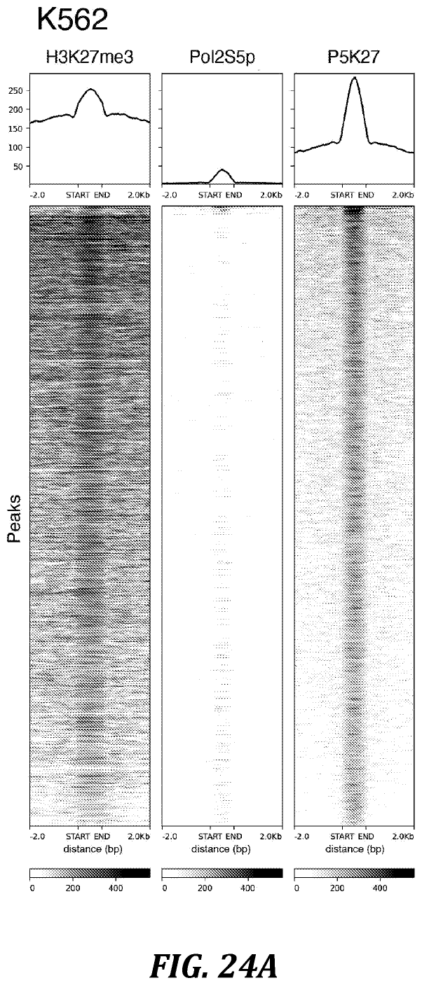

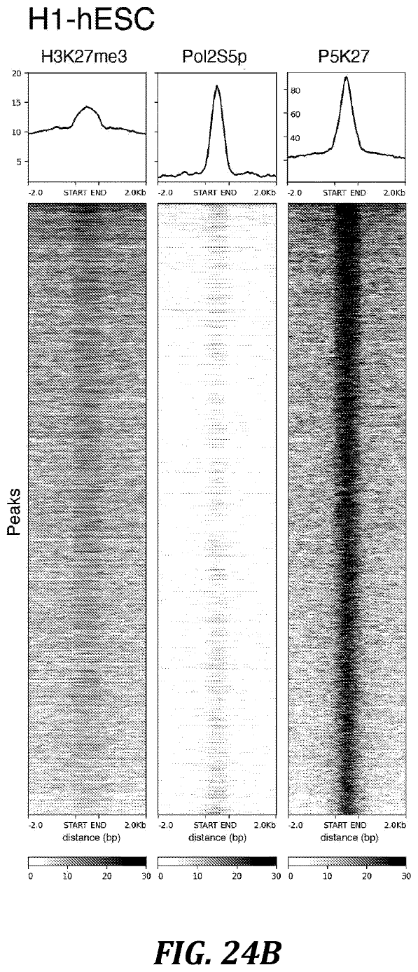

A fraction of genomic sites were identified as peaks in both Pol2S5p and H3K27me3. If the overlaps of a H3K27me3 peak with Pol2S5p is less than 50% of the H3K27me3 peak span, we resolve the region as a H3K27me3 peak (and vice-versa for Pol2S5p peaks). The remainder of the peaks are termed as overlap peaks (FIGS. 24A and 24B) and were not used in the analysis.

Implementation Details

Since the GP employs the covariance between cut sites, the memory demand grows approximately quadratic with the number of unique cut sites. However, cuts that are further apart than 10,000 base pairs, express no relevant covariance and must not be considered in the same GP. This observation is used to split up genomic regions into intervals with at most 10,000 unique cut sites. Each interval with an additional 10,000 bases on either side are padded to ensure stable estimation of the signal at the interval boundaries and discard the padding after deconvolution. A GP is fit separately for each interval and the results concatenated to obtain a deconvolution of all genomic regions.

We also limit the deconvolution to regions where the Gaussian kernel density estimate of all cuts (bandwidth=200) indicates at least 2 cuts per 100 base pairs. Neighboring regions are merged if they are separated by fewer than 10,000 base pairs. These selected regions were segmented into intervals as described above. All intervals of the selected regions were grouped into ˜200 groups and applied the posteriori point maximization of PyMC3 (Salvatier et al., (2016). Probabilistic programming in python using pymc3. Peer J Computer Science 2: e55) for deconvolution.

Application to Single-Cell Data

The reads of all cells of the two cell types from both replicates were aggregated into a pseudo-bulk set of fragments for each cell type. After applying the 2for1separator algorithm to identify Pol2S5p and H3K27me3 peaks, FeatureCount (Liao et al., (2013). Featurecounts: An efficient general purpose program for assigning sequence reads to genomic features. Bioinformatics 30: 923-930) was used to count the number of fragments that overlap each peak for each cell and target.

The resulting count vectors were corrected for library size and the normalized vector for H3K27me3 and Pol2S5p were concatenated to produce a normalized peak count vector per cell. The normalized data were log-transformed.

Principal Components Analysis was first applied to the normalized and log-transformed data and used 50 components for downstream analysis. Since the first or second principal component remains very strongly correlated with the library size despite the normalization, the respective component in the UMAP and other downstream analyses were excluded.

### Data and Code Availability

All software required for implementation of computational methods described herein are provided online at github.com/FredHutch. Other software is provided at github at mpmeers/MeersEtAl_MulTI-Tag and FredHutch/SEACR. All sequencing data have been deposited in GEO under ID code GEO: GSE183032.

While illustrative embodiments have been illustrated and described, it will be appreciated that various changes can be made therein without departing from the spirit and scope of the invention.

1. An in situ method for detecting a site of DNA accessibility in the chromatin of a cell, comprising:
contacting a permeabilized cell with a first affinity reagent that specifically binds a nucleosome depleted region (NDR) marker, wherein the first affinity reagent is coupled to at least one transposome comprising:
at least one transposase; and
a transposon comprising:
a first DNA molecule comprising a first transposase recognition site; and
a second DNA molecule comprising a second transposase recognition site;
activating the at least one transposase under low ionic conditions, thereby cleaving and tagging chromatin DNA with the first and second DNA molecules and excising a tagged DNA segment associated with the NDR marker;
isolating the excised tagged DNA segment; and
determining the nucleotide sequence of the excised tagged DNA segment, thereby detecting the site of DNA accessibility in the chromatin of the cell.

2. The method of claim 1, wherein the first affinity reagent is directly coupled to at least one transposase.

3. The method of claim 2, wherein the first affinity reagent and transposase are disposed in a fusion protein.

4. The method of claim 1, wherein the first affinity reagent is indirectly coupled to the at least one transposase.

5. The method of claim 4, wherein the transposase is linked to a specific binding agent that specifically binds the first affinity reagent.

6. The method of claim 4, further comprising:
contacting the cell with a second affinity reagent that specifically binds the first affinity reagent, and wherein the transposase is linked to a specific binding agent that specifically binds the second affinity reagent.

7. The method of claim 4, further comprising:
contacting the cell with a second affinity reagent that specifically binds the first affinity reagent;
contacting the cell with a third affinity reagent that specifically binds the second affinity reagent,
and wherein the transposase is linked to a specific binding agent that specifically binds the third affinity reagent.

8. The method of claim 5, wherein the specific binding agent comprises protein A or protein G or a fourth affinity reagent that specifically binds the first affinity reagent, the second affinity reagent and/or the third affinity reagent.

9. The method of claim 1, wherein the first, second, and/or third affinity reagents independently is or comprises an antibody, an antibody-like molecule, a DARPin, an aptamer, a chromatin-binding protein, other specifically binding molecule, or a functional antigen-binding domain thereof.

10. The method of claim 9, wherein the antibody-like molecule is an antibody fragment and/or antibody derivative, optionally a single-chain antibody, a bispecific antibody, an Fab fragment, an F(ab)₂ fragment, a V_{H}H fragment, a V_{NAR} fragment, or a nanobody, optionally wherein the single-chain antibody is a single chain variable fragment (scFv), or a single-chain Fab fragment (scFab).

11-12. (canceled)

13. The method of claim 1, wherein the low ionic conditions are characterized by monovalent ionic concentration of less than about 10 mM.

14. The method of claim 13, wherein the low ionic conditions are obtained by diluting liquid conditions of the transposase with a Mg⁺⁺ solution, removing liquid supernatant from the transposase and replacing it with a low ionic strength solution, and/or conducting a stringent (e.g., 300 mM) wash followed by adding a low ionic strength solution.

15. The method of claim 1, further comprising contacting the permeabilized cell with a polar compound prior to or during the step of activating the transposase under low ionic conditions, optionally wherein the polar compound is 1,6-hexanediol or N,N-dimethylformamide.

16. (canceled)

17. The method of claim 1, wherein the cell is immobilized on a solid surface, optionally wherein the solid surface comprises a bead or wall of a microtiter plate.

18. (canceled)

19. The method of claim 1, wherein the first and/or second DNA molecule further comprises a barcode, a sequencing adaptor, and/or a universal priming site.

20-21. (canceled)

22. The method of claim 1, wherein the at least one transposase comprises a Tn5 transposase.

23. The method of claim 22, wherein activating the transposase under low ionic conditions comprises contacting the transposase with Mg⁺⁺, optionally with about 0.1 mM Mg⁺⁺ to about 10 mM Mg⁺⁺.

24. The method of claim 1, wherein the at least one transposase comprises a Mu transposase, an IS5 transposase, or an IS91 transposase.

25. (canceled)

26. The method of claim 1, wherein the least one transposome comprises at least two different transposases, and wherein the different transposases integrate different DNA sequences into the chromatin DNA.

27. The method of claim 1, wherein the method is performed with a plurality of first affinity reagents, thereby producing a plurality of excised tagged DNA segments, and wherein the method further comprises isolating a plurality of excised tagged DNA segments, and optionally further comprising analyzing the isolated tagged DNA segments, optionally wherein analyzing the isolated tagged DNA segments comprises determining the nucleotide sequence of the tagged DNA segments, optionally using sequencing or hybridization techniques with or without amplification.

28-33. (canceled)

34. The method of claim 1, wherein the cell and/or the nucleus of the cell is permeabilized by contacting the cell with digitonin.

35. The method of claim 1, further comprising subjecting the excised DNA to salt fractionation.

36. The method of claim 1, wherein the NDR marker is a histone modification, optionally methylated H3K4, optionally wherein methylated H3K4 is bi-methylated or tri-methylated, optionally wherein the NDR marker is an initiating form of RNA Polymerase II, optionally serine 5-phosphorylated RNA Polymerase II (RNAPIIS5P) or serine 2-phosphorylated RNA Polymerase II (RNAPIIS2P).

37. (canceled)

38. The method of claim 1, further comprising contacting the permeabilized cell with a known amount of spike-in DNA configured to facilitate calibration, optionally wherein the spike-in DNA is or comprises exogenous DNA, exogenous chromatin, or recombinant nucleosomes.

39. (canceled)

40. The method of claim 38, wherein the first affinity reagent is coupled to a plurality of transposomes, a fraction of the plurality of transposomes comprising a known amount of spike-in DNA, and wherein the spike-in DNA can be used for calibration.

41. The method of claim 1, wherein the at least one transposome comprises a fusion protein comprising a first domain comprising a Tn5 transposase domain and second domain comprising a protein A domain, a protein G domain, or a protein A/G hybrid domain.

42. The method of claim 1, wherein the method is performed for a plurality of cells and the method further comprises mapping the determined sequences of one or more excised tagged DNA segments to a consensus genome of the plurality of the cells.

43. The method of claim 1, further comprising mapping the determined sequence of the excised tagged DNA segment to the genome of the cell.

44. The method of claim 1, wherein the method is performed for a plurality of cells, wherein the excised tagged DNA segments of each of the plurality of cells is tagged with a cell-specific barcode or combination of barcodes that is unique to each cell, optionally
wherein the method further comprises application of combinatorial indexing to provide the cell-specific barcode or combination of barcodes to the excised tagged DNA segments of each of the plurality of cells and/or
wherein the plurality of cells is disposed in a three-dimensional arrangement and the cell-specific barcode or combination of barcodes is unique to a location in the three-dimensional arrangement, optionally wherein the three-dimensional arrangement is a tissue slice or tissue culture array.

45-47. (canceled)

48. A method of detecting active and repressive regulomes in a cell, comprising performing the method recited in claim 1,
wherein the method comprises contacting the permeabilized cell with the first affinity reagent in combination with a fifth affinity reagent that specifically binds a repressive regulatory element marker, wherein the fifth affinity reagent is coupled to at least one transposome comprising:
at least one transposase; and
a transposon comprising:
a first DNA molecule comprising a first transposase recognition site; and
a second DNA molecule comprising a second transposase recognition site;
activating the at least one transposase under low ionic conditions, thereby cleaving and tagging chromatin DNA with the first and second DNA molecules and excising a tagged DNA segment associated with the repressive regulatory element marker;
isolating the excised tagged DNA segment associated with the repressive regulatory element marker;
determining the sequence of the excised tagged DNA segment associated with the repressive regulatory element marker; and
deconvoluting the sequences determined from the excised tagged DNA segment associated with the NDR marker and the excised tagged DNA segment associated with the repressive regulatory marker, thereby detecting active and repressive regulomes in the cell.

49-50. (canceled)

51. A method for preparing a library of excised chromatin DNA comprising the method of claim 1.

52-53. (canceled)
